(19) United States
(12) Patent Application Publication
(10) Pub. No.: US 2025/0255956 A1
(43) Pub. Date: Aug. 14, 2025

(54) DUAL AND TRIPLE HAPTEN CONJUGATES, COMPOSITIONS, PROCESSES FOR MAKING, AND METHODS OF TREATMENT THEREWITH

(71) Applicant: PURDUE RESEARCH FOUNDATION, West Lafayette, IN (US)

(72) Inventors: Philip Low, West Lafayette, IN (US); Ananda Kumar Kanduluru, West Lafayette, IN (US); Madduri Srinivasarao, West Lafayette, IN (US); Imrul Shahriar, West Lafayette, IN (US); Mohini Kamra, West Lafayette, IN (US)

(21) Appl. No.: 18/858,284

(22) PCT Filed: Apr. 19, 2023
(86) PCT No.: PCT/US2023/065924

§ 371 Date: Oct. 18, 2024

Related U.S. Application Data
(60) Provisional application No. 63/429,030, filed on Nov. 30, 2022.
(60) Provisional application No. 63/392,744, filed on Jul. 27, 2022.
(60) Provisional application No. 63/332,521, filed on Apr. 19, 2022.

(51) Int. Cl. A61K 39/385; A61K 39/00; A61K 39/395; A61K 47/54; A61K 47/55; A61P 31/16; A61P 35/00

(52) U.S. Cl. CPC A61K 39/385; A61K 39/3955; A61K 47/545; A61K 47/549; A61K 47/551; A61P 31/16; A61P 35/00; A61K 2039/505; A61K 2039/542; A61K 2039/585; A61K 2039/6012

(57) ABSTRACT

A conjugate having the formula TL-L-Hn, wherein TL is a targeting ligand for a target protein on the surface of a virus, a virus-infected cell, a cancer cell, an immune cell, or a fibroblast; L is a linker; H is a hapten; and n is an integer of 2 or greater. Compositions comprising the conjugate and methods of use are also provided.

### PRIORITY

This application is related to and claims the priority benefit of (1) U.S. Provisional Patent Application No. 63/332,521 filed Apr. 19, 2022; (2) U.S. Provisional Application No. 63/392,744 filed Jul. 27, 2022; and (3) U.S. Provisional Application No. 63/429,030 filed Nov. 30, 2022. The contents of the aforementioned applications are hereby incorporated by reference in their entireties into this disclosure.

### TECHNICAL FIELD

The present disclosure includes conjugates comprising two or more haptens linked to a targeting ligand for a target protein on a virus or cell, as well as compositions, such as pharmaceutical compositions, comprising such conjugates and processes for making such conjugates. Additionally, the present disclosure further includes methods of treating viral infections, fibrosis, and cancer.

### BACKGROUND

This section introduces aspects that may help facilitate a better understanding of the disclosure. Accordingly, these statements are to be read in this light and are not to be understood as admissions about what is or is not prior art.

Well-known Fc-mediated (fragment crystallizable domain-mediated) antibody effector functions are antibody-dependent cell-mediated cytotoxicity (ADCC), antibody-dependent cellular phagocytosis (ADCP), and complement-dependent cytotoxicity (CDC). In addition, antibodies have been found to mediate inflammation and immunomodulation through the induction of cellular differentiation and activation. These mechanisms can either protect viral replication or enhance infected cell clearance through the antibody-mediated effector functions against virally infected cells, such as viral glycoprotein shedding, viral glycoprotein internalization, antibody cooperativity, and antibody glycosylation. A family of receptors that recognize the Fc domain of IgG (Immunoglobin G) molecules is known as the FcRn and FcγRs family. Several studies have established a correlation between Fc:FcγRs affinities and selectivity and the cytotoxic functions of immune effector cells engaged by immune complexes (ICs).

The human gamma receptors (FcγRs) include FcγRI (CD64), FcγRIIa (CD32a), FcγRIIb (CD32b), FcγRIIc (CD32c), FcγRIIIa (CD16a), and FcγRIIIb (CD16b), and these receptors are expressed at different levels on the surfaces of various immune cells. FcγRIIIa is a key surface receptor in terms of its contribution to ADCC activity and is found on the surfaces of natural killer (NK) cells, macrophages, monocytes, mast cells, eosinophils, and dendritic cells. However, it is the only FcγR expressed by NK cells.

Humans express two FcγRIIIa allotypes that differ in a single amino acid at position 158; the residue can be either valine (V) or phenylalanine (F), whereby the isoform with V at position 158 has high affinity for the Fc domain of IgG1, and the isoform with F at position 158 has low affinity. Engagement of the high affinity FcγRIIIa-V158 bp immune complexes (ICs) results in a stronger in vitro cytotoxic potency than that of FcγRIIIa-F158. Fc-engineered antibodies with improved affinity to FcγRIIIa show higher therapeutic efficacy compared to native Fc because they can prime and activate NK cells more efficiently. ADCP activity is known to be triggered by FcγRIIa intracellular signaling, which has been shown by glycoengineered antibodies to exhibit enhanced affinity toward FcγRIIa, resulting in increased ADCP activity. Mainly, Fc variants displaying high affinity for the FcγRIIa-R131 isoform and high selectivity for FcγRIIa over FcγRIIb have been shown to mediate improved ADCP activity. The mechanism of phagocytes can be engaged by either complement receptor. Infected cells can also be eliminated by CDC as well as by ADCC and/or ADCP mediated by FcγR bearing effector cells.

Almost all FDA-approved, therapeutic antibodies are full-size IgG1 antibodies about 150 kDa in size. Dimitrov, Engineered CH2 domains (nanoantibodies), mAbs 1(1): 26-28 (2009). The same is true for the vast majority of antibodies in clinical trials. The few exceptions are antigen binding fragments (Fabs).

Full-size antibodies present fundamental problems for therapy. One problem is poor tissue penetration, such as poor solid tumor penetration. Dimitrov (2009), supra. Another problem is poor or no binding to regions on the surfaces of some molecules, such as the envelope glycoprotein of human immunodeficiency virus (HIV). Id.

In view of the foregoing, what is needed are materials and methods to bring antibodies (Abs), whether naturally occurring, exogenously administered autologous Abs, or exogenously administered IgG Ab, into close proximity with viruses, virally infected cells, cancer cells, immune cells, and/or fibroblasts, for example, to improve Ab function. This and other objects and advantages, as well as inventive features, will be apparent from the detailed description provided herein.

### SUMMARY

In one aspect of the disclosure, provided are conjugates of the formula TL-L-Hₙ, and pharmaceutically acceptable salts thereof, wherein TL is a targeting ligand for a target protein on the surface of a virus, a virus-infected cell, a cancer cell, an immune cell, or a fibroblast; L is a linker; H is a hapten; and n is an integer, which is either 2 or 3.

In a further aspect of the disclosure, provided are conjugates having the following Formula IA:

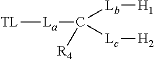

and pharmaceutically acceptable salts thereof, wherein:
TL is a targeting ligand for a target protein on the surface of a virus, a virus-infected cell, a cancer cell, an immune cell, or a fibroblast; Lₐ, L_{b}, and L_{c} are linkers; C is a carbon atom;
R₄ is selected from hydrogen, C₁-C₅ alkyl, C₁-C₅ alkenyl, or C₁-C₅ alkvnl group; H₁ and H₂ are haptens.

In an additional aspect of the disclosure, provided are conjugates having the following Formula IB:

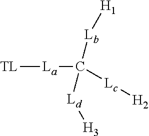

and pharmaceutically acceptable salts thereof, wherein TL is a targeting ligand for a target protein on the surface of a virus, a virus-infected cell, a cancer cell, an immune cell, or a fibroblast; Lₐ, L_{b}, L_{c} and L_{d} are linkers; C is a carbon atom; and H₁, H₂, and H₃ are haptens.

In an additional aspect of the disclosure, conjugates having the structure of Formula II are provided:

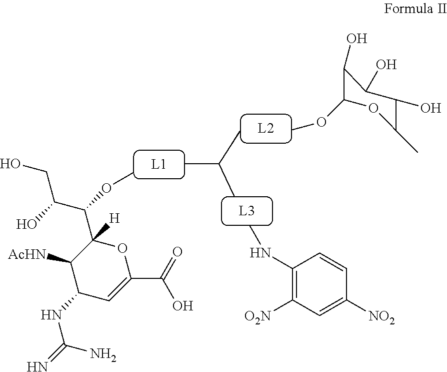 [SMILES: COC1OC(C)C(O)C(O)C1O ; CO[C@H]([C@@H]1OC(C(=O)O)=C[C@H](NC(=N)N)[C@H]1NC(C)=O)[C@H](O)CO ; CNc1ccc([N+](=O)[O-])cc1[N+](=O)[O-]]

or pharmaceutical salts thereof, wherein L1, L2 and L3 are each, independently, linkers.

In certain embodiments, the conjugate has the formula:

TL-L-Hₙ

or is a pharmaceutically acceptable salt thereof, wherein: TL is a targeting ligand for a target protein on the surface of a virus, a virus-infected cell, a cancer cell, an immune cell, or a fibroblast; L is a linker; H is a hapten; and n is an integer of 2-3; and optionally, wherein at least two of the Hs can each bind a different antibody when brought into contact therewith.

At least two of the Hs can each be bound by an antibody. Each H can be bound by a different antibody. In certain embodiments, at least two of the Hs can each bind a different antibody when brought into contact with antibodies in vivo. In certain embodiments, at least two of the Hs can each bind a different antibody when brought into contact with antibodies in vitro.

Each H can be independently selected from a rhamnose fragment, an α-galactosyl moiety, a dinitrophenyl fragment, a trinitrophenyl fragment, or a combination thereof. At least one H can be an influenza virus antigen selected from haemagglutinin and neuraminidase. At least one H can be a hepatitis antigen selected from L-HBsAg, S-HBsAg, M-HBsAg, and preS. At least one H can be gp120 or gp160. At least one H can be a glycoprotein.

n can be 2. n can be 3.

Each H can be independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

In certain embodiments, n is 2, a first H is a DNP fragment, and a second H is a rhamnose fragment.

The target protein can be an envelope protein of a virus or a viral envelope protein on the surface of a virus-infected cell. The target protein can be influenza neuraminidase or influenza hemagglutinin. The target protein can be a respiratory syncytial virus fusion protein F. The c target protein can be coronavirus spike protein. The target protein can be hepatitis B virus surface antigen or HBV core antigen. The target protein can be a cell-surface receptor on a cancer cell. The target protein can be a folate receptor. The target protein can be folate receptor α or folate receptor β. The target protein can be a prostate-specific membrane antigen.

The conjugate of any one of claims 1-3, 5, 6, or 9-12, wherein the target protein is carbonic anhydrase 9. The target protein can be a luteinizing hormone releasing hormone receptor. The target protein can be a neurokinin 1 receptor. The target protein can be a cell-surface receptor on a tumor-associated macrophage. The target protein can be a cell-surface receptor on a myeloid-derived suppressor cells. The target protein can be a cell-surface receptor on a cancer-associated fibroblast. The target protein can be a fibroblast activation protein. The targeting ligand can be a neuraminidase inhibitor. The targeting ligand can be an oseltamivir fragment, a zanamivir fragment, a peramivir fragment, or a laninamivir fragment. The targeting ligand can be a zanamivir fragment. The targeting ligand can be a folic acid fragment or an analog thereof. The targeting ligand can be 5-methyltetrahydrofolate.

L can comprise (—CH₂CH₂—O—)ₙ, where n is an integer between and including 1 and 32, a peptide, a peptidoglycan, or a combination of two or more of the foregoing. L can be a branched linker and at least two of the haptens are connected to different branches of the linker, wherein the different branches optionally extend from different atoms of the linker.

The targeting ligand can be a folic acid fragment or a derivative thereof, at least a first H can comprise a rhamnose fragment, and at least a second H can comprise a dinitrophenyl fragment.

The conjugates hereof can be formulated as a prodrug.

In certain embodiments, the conjugate has the formula

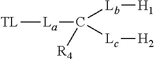

or is a pharmaceutically acceptable salt thereof, wherein: TL is a targeting ligand for a target protein on the surface of a virus, a virus-infected cell, a cancer cell, an immune cell, or a fibroblast; Lₐ, L_{b}, and L_{c} are each a linker, which can be the same or different; C is a carbon atom; R₄ is selected from a hydrogen, C₁-C₅ alkyl, C₁-C₅ alkenyl, or C₁-C₅ alkynl group; H₁ and H₂ are each a hapten; and, optionally, wherein H₁ and H₂ each can bind a different antibody.

In certain embodiments, the conjugate has the formula

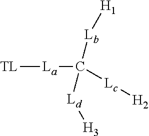

or is a pharmaceutically acceptable salts thereof, wherein: TL is a targeting ligand for a target protein on the surface of a virus, a virus-infected cell, a cancer cell, an immune cell, or a fibroblast; Lₐ, L_{b}, L_{c} and L_{d} are each a linker, which can be the same or different; C is a carbon atom; H₁, H₂, and H₃ are each a hapten; and, optionally, wherein each H₁, H₂ and H₃ can each bind a different antibody.

H₁ and H₂ can each be bound by an antibody. H₁, H₂, and H₃ can each be bound by an antibody. H₁ and H₂ can each independently be selected from a rhamnose fragment, an ci-galactosyl moiety, a dinitrophenyl fragment, a trinitrophenyl fragment, or a combination thereof. H₁, H₂ and H₃ can each be independently selected from a rhamnose fragment, an α-galactosyl moiety, a dinitrophenyl fragment, a trinitrophenyl fragment, or a combination thereof. H₁ or H₂ can each be independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus. H₁, H₂ or H₃ can each be independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus. In certain embodiments, H₁ is a DNP fragment and H₂ is a rhamnose fragment.

At least one hapten can be an influenza virus antigen selected from haemagglutinin and neuraminidase. At least one hapten can be a hepatitis antigen selected from L-HBsAg, S-HBsAg, M-HBsAg, and preS. At least one hapten can be gp120 or gp160. At least one hapten can be a glycoprotein.

The target protein can be an envelope protein of a virus or a viral envelope protein on the surface of a virus-infected cell. The target protein can be influenza neuraminidase or influenza hemagglutinin. The target protein can be a respiratory syncytial virus fusion protein F. The target protein can be a coronavirus spike protein. The target protein can be hepatitis B virus surface antigen or HBV core antigen. The target protein can be a cell-surface receptor on a cancer cell. The target protein can be a folate receptor. The target protein can be folate receptor α or folate receptor β. The target protein can be a prostate-specific membrane antigen. The target protein can be carbonic anhydrase 9. The target protein can be a luteinizing hormone releasing hormone receptor. The target protein can be a neurokinin 1 receptor. The target protein can be a cell-surface receptor on a tumor-associated macrophage. The target protein can be a cell-surface receptor on a myeloid-derived suppressor cells. The target protein can be a cell-surface receptor on a cancer-associated fibroblast. The target protein can be a fibroblast activation protein. The targeting ligand can be a neuraminidase inhibitor. The targeting ligand can be an oseltamivir fragment, a zanamivir fragment, a peramivir fragment, or a laninamivir fragment. The targeting ligand can be a zanamivir fragment. The targeting ligand can be a folic acid fragment or an analog thereof. The targeting ligand can be 5-methyltetrahydrofolate.

In certain embodiments of the conjugate, at least one of Lₐ, L_{b}, L_{c}, and L_{d} each independently comprise: (—CH₂CH₂—O—)ₙ, where n is an integer between and including 1 and 32, an alkyl group, a peptide, a peptidoglycan, or a combination of two or more of the foregoing. In certain embodiments of the conjugate, at least one of Lₐ, L_{b}, and L_{c}, each independently comprise: (—CH₂CH₂—O—)ₙ, where n is an integer between and including 1 and 32, an alkyl group, a peptide, a peptidoglycan, or a combination of two or more of the foregoing. n can be an integer between and including 1 and 16.

At least one of Lₐ, L_{b}, and L_{c}, and L_{d} can comprise a peptide fragment or a peptidoglycan fragment. At least one of Lₐ, L_{b}, and L_{c} can comprise a peptide fragment or a peptidoglycan fragment. Lₐ, L_{b} and L_{c} L_{d} can each independently comprise a C₂-C₁₈ alkyl group. Lₐ, L_{b} and L_{c} can each independently comprise a C₂-C₁₈ alkyl group.

In certain embodiments, a conjugate has the formula:

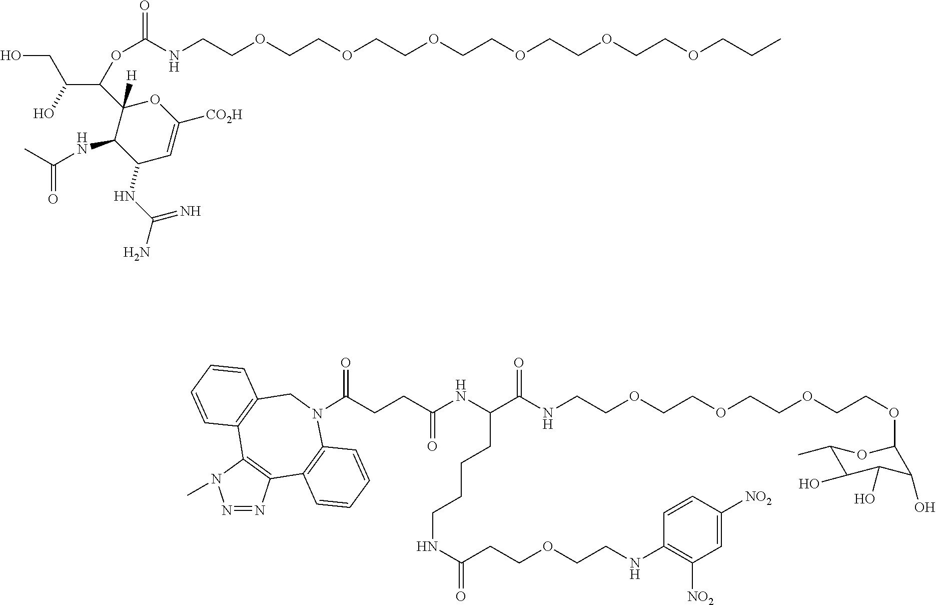

or is a pharmaceutically acceptable salt thereof.

In certain embodiments, the conjugate has the formula:

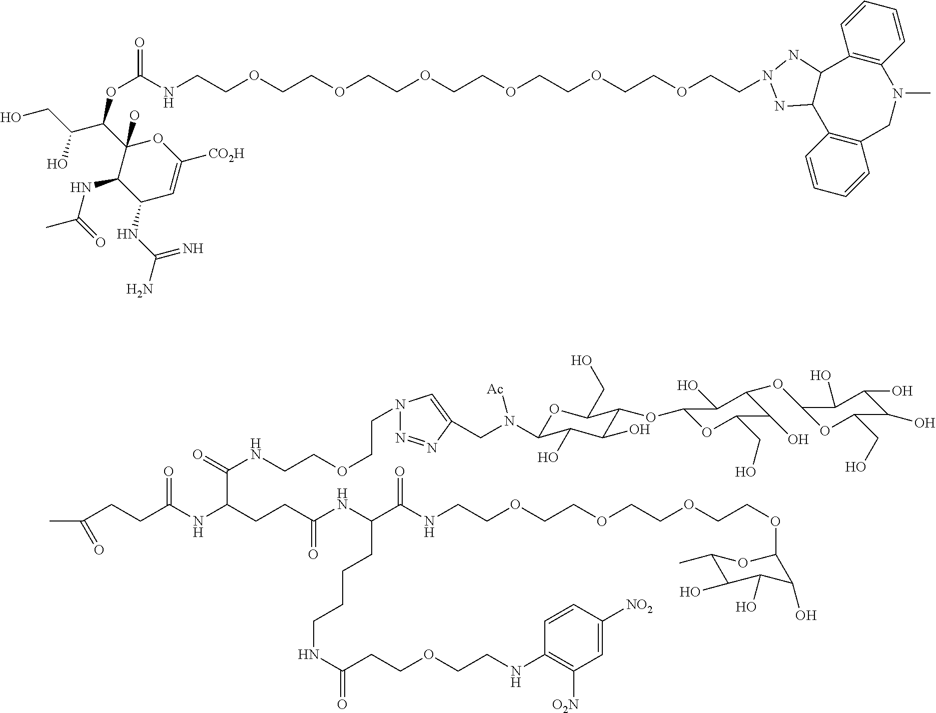

or is a pharmaceutically acceptable salt thereof.

One or more of the —OH groups of the conjugate can be independently replaced with a thiol, a phosphate, or a phosphanate ester. One or more of the —OH groups can be replaced with —OC(═O)R, wherein R is an alkyl group. One or more of the —OH groups can be replaced with —OC(═O)R, wherein R is a C₁-C₆ alkyl group. The amine (—NH₂) group can be replaced with —OC(═O)R₂, and R₂ can be an alkyl group. The amine (—NH₂) group can be replaced with —OC(═O)R₂, and R₂ can be a C₁-C₆ alkyl group. The carboxyl (—COOH) group can be replaced with —OC(═O)R₃, wherein R₃ is an alkyl group. The carboxyl (—COOH) group can be replaced with —OC(═O)R₃, wherein R₃ is a C₁-C₆ alkyl group.

In certain embodiments, the conjugate has the formula:

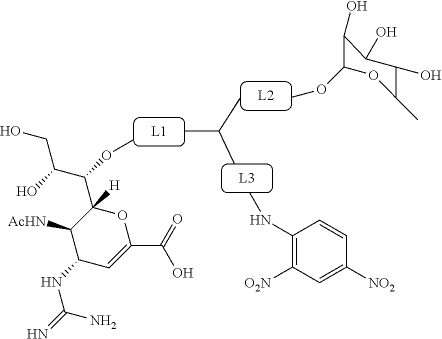 [SMILES: COC1OC(C)C(O)C(O)C1O ; CO[C@H]([C@@H]1OC(C(=O)O)=C[C@H](NC(=N)N)[C@H]1NC(C)=O)[C@H](O)CO ; CNc1ccc([N+](=O)[O-])cc1[N+](=O)[O-]]

or is a pharmaceutically acceptable salt thereof, wherein L1, L2 and L3 are linkers. One or more of L1, L2 and L3 can comprise (—CH₂CH₂—O—)ₙ wherein n can be an integer between and including 1 and 16. L1, L2 and L3 can each independently comprise a C₂-C₁₈ alkyl group, a peptide fragment, or a peptidoglycan fragment.

In certain embodiments, the conjugate has the formula.

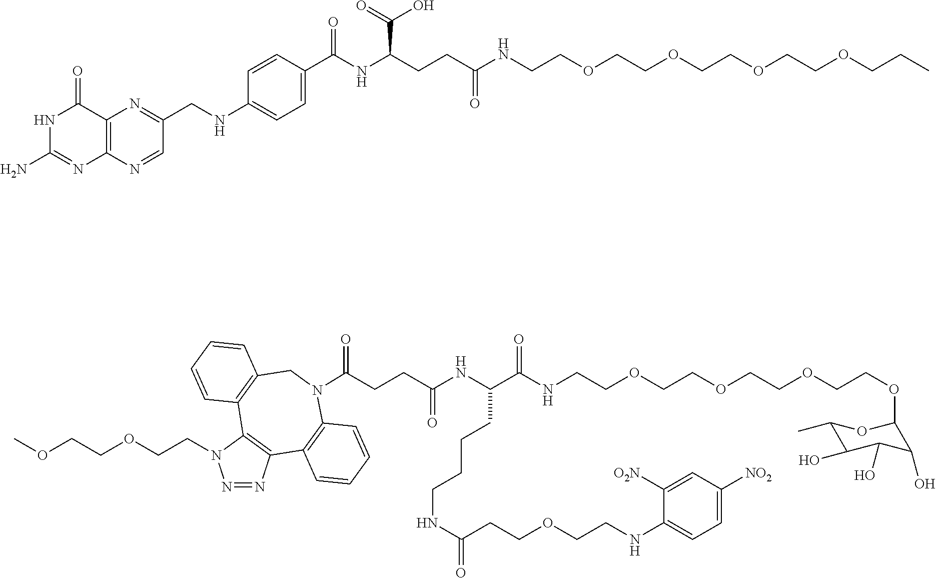

or is a pharmaceutically acceptable salt thereof.

The conjugate can further be complexed to one or more antibodies in vivo.

A pharmaceutical composition is also provided. The pharmaceutical composition can comprise a conjugate (e.g., any of the conjugates hereof) and a pharmaceutically acceptable excipient.

A method of treating a viral infection in a subject is also provided. The method of treating a viral infection in a subject can comprise administering to the subject an effective amount of a conjugate hereof or a pharmaceutical composition hereof. The method can further comprise administering to the subject autologous antibodies or allogeneic immunoglobulin G (IgG) antibodies. The viral infection can be influenza. The conjugate or the pharmaceutical composition can be administered orally. The conjugate or the pharmaceutical composition can be administered once daily. The conjugate or the pharmaceutical composition can be administered more than once daily. The conjugate or the pharmaceutical composition can be administered twice daily.

A method of treating cancer in a subject is provided. The method of treating cancer in a subject can comprise administering to the subject an effective amount of a conjugate hereof or a composition (e.g., pharmaceutical composition) hereof. The method can further comprise administering to the subject autologous antibodies or allogeneic IgG antibodies. The cancer can be a hot cancer. The cancer can be renal cancer, lung cancer, or colorectal cancer.

The conjugate or the pharmaceutical composition can be administered orally or intravenously. The conjugate or pharmaceutical composition can be administered once daily.

The method of treating cancer can further comprise administering a second therapy to the subject, wherein the second therapy comprises chemotherapy, sunitinib, a PD-1 inhibitor, or a PDL-1 inhibitor.

A method for activating an immune response in a subject is also provided and, in certain embodiments, such method comprises administering to the subject an effective amount of a conjugate hereof or a pharmaceutical composition hereof. The immune response can be an innate immune response. The immune response can be activated in a targeted area of the subject, for example, such as a tumor microenvironment or a location of a virus replication site.

The method for activating an immune response can further comprise administering to the subject autologous antibodies or allogeneic IgG antibodies. Administration of the effective amount of the conjugate or the pharmaceutical composition can induce reprogramming of M2-type macrophages to M1-type macrophages in the targeted area.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1A is a schematic explanation of antibody recruitment.

FIG. 1B is a schematic explanation of antibody recruitment using embodiments of the conjugates hereof, wherein 10 comprises a targeting ligand (TL) and two haptens (each H), the targeting ligand is specific to neuraminidase of a virus or a virus-infected cell (i.e., the receptor), and the haptens H of the conjugate 10 bind human endogenous anti-DNP and anti-Rha antibodies (Abs 12).

FIGS. 2A-2H show liquid chromatography-mass spectrometry (LC-MS), mass spectrometry, and ultraviolet spectrometry data of Compound 7 (FIG. 2A), Compound 8 (FIG. 2B), Compound 9 (FIG. 2C) Compound 12 (FIG. 2D), Compound 18 (FIG. 2E), Compound 22 (FIG. 2F), Compound 23 (FIG. 2G), and Compound 24 (FIG. 2H).

FIG. 3 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX©-C) at 24 hours post-infection (hpi), and administration of conjugate at 48 hpi.

FIG. 4 is a graph of days after infection vs. body weight (%) for the mice treated as described in FIG. 4.

FIG. 5 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX©-C) at 24 hpi, and administration of conjugate at 48 hpi.

FIG. 6 is a graph of days after infection vs. body weight (%) for the mice treated as described in FIG. 5.

FIG. 7 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX®-C) at 24 hpi, and administration of conjugate at 96 hpi.

FIG. 8 is a graph of days after infection vs. body weight (%) for the mice treated as described in FIG. 7.

FIG. 9 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX®-C) at 24 hpi, and administration of conjugate at 48 hpi.

FIG. 10 is a graph of days after infection vs. body weight (%) for the mice treated as described in FIG. 9.

FIG. 11 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX®-C) at 24 hpi, and administration of conjugate at 96 hpi.

FIG. 12 is a graph of days after infection vs. body weight (%) for the mice treated as described in FIG. 11.

FIG. 13A shows the viral titer (fold change) for conjugate and commercially available drugs 48 hpi.

FIG. 13B shows the viral titer (fold change) for conjugate and commercially available drugs 96 hpi.

FIG. 14A shows the viral titer (fold change) for conjugate and commercially available drugs 48 hpi.

FIG. 14B shows the viral titer (fold change) for conjugate and commercially available drugs 96 hpi.

FIG. 15A is a graph of days after infection vs. % survival.

FIG. 15B is a graph of days after infection vs. body weight (%).

FIG. 16 is a bar graph of route of administration (SC=subcutaneous; OG=oral gavage; IV=intravenous; IN=intranasal; PBS=phosphate-buffered saline) vs. viral titer (PFU/ml per ng of ribonucleic acid (RNA)).

FIG. 17 is a bar graph of OG and PBS vs. viral titer (PFU/ml per ng of RNA).

FIG. 18 is a graph of time (h=hours) vs. concentration (ng/mL) for intravenous (IV) administration of a single dose of 1.5 μmol/kg (n=2 mice/timepoint) that relates to a pharmacokinetics study of dosing of Compound 24 (Zan-DNP-Rhamnose).

FIG. 19 is a graph of time (h=hours) vs. concentration (ng/mL) for oral administration of a single dose of 4.5 mol/kg (n=2 mice/timepoint) that relates to a pharmacokinetics study of dosing of Compound 24 (Zan-DNP-Rhamnose).

FIG. 20A is a graph of days after infection vs. % survival that relates to a dose escalation study, where the mice were infected with 100×LD₅₀ of Influenza A/H1N1/PR8/1934, hIVIg was used as the source of anti-hapten antibodies (injected at a dose of 8 g/kg at 24 hours prior to drug administration), and doses of Compound 24 (Zan-DNP-Rhamnose) were administered at 48 hpi with a single dose of 1.5 μmol/kg zan-DNP-Rhamnose (IV) or two doses of 4.5 umol/kg zan-DNP-rhamnose (oral).

FIG. 20B is a graph of days after infection vs. % body weight that relates to the dose escalation study of FIG. 20A, and the legend in FIG. 20A also applies to FIG. 20B.

FIG. 21 is a graph of days post-infection for PBS and IV and OG administration of zanamivir-DNP-rhamnose (Compound 24) vs. viral titer (PFU/ml per ng of RNA).

FIG. 22 is a graph of days after infection vs. survival (%).

FIG. 23 is a graph of days after infection vs. body weight (%).

FIG. 24 is a graph showing lung titers in Balb/c mice 24 hours after treatment, which in turn is 48 hours after infection with 10 LD₅₀ A/H1N1/PR8/1934 virus (at day 0), comparing treatment with Compound 24 with PBS, Tamiflu®, Xofluza®, and a cohort of mice that were not infected (no infection).

FIG. 25 is a graph showing survival of Balb/c mice after infection with 10 LDso A/H1N1/PR8/1934 virus (at day 0) and dosing of Compound 24 (C.24) at day 4 after infection by various routes and compared with PBS only; namely, IN route, IV route, and OG route.

FIG. 26 is a graph showing infection vs. % body weight based on the dosing data in FIG. 25.

FIGS. 27A and 27B show graphs of the concentration of Compound 24 (in nM) versus percent killing via antibody dependent cellular cytotoxicity (ADCC) and via complement dependent cytotoxicity (CDC), respectively, executed against N1-transfected (NA-HEK) and wild-type (WT) HEK 293 cells in vitro.

FIGS. 28A and 28B show graphs of days after infection vs. survival (%), and days after infection vs. body weight (%), respectively, from a study on dose escalation of IV administered Compound 24 in Balb/c mice.

FIGS. 29A and 29B show graphs of days after infection vs. survival (%), and days after infection vs. body weight (%), respectively, from a study comparing administration of the conjugates hereof as compared to mono-haptens (96 hpi treatment) in Balb/c mice.

FIGS. 30A and 30B show graphs of days after infection vs. survival (%), and days after infection vs. body weight (%), respectively, from a study comparing administration of the conjugates to Balb/c mice infected with various strains of flu, where (A) is the Influenza A/California/07/2009 (H1N1)pdm09+Compound 24 treatment group; (B) is the Influenza A/Wisconsin/67/2005 (H3N2)+Compound 24 treatment group; (C) is the Influenza B/Florida/04/2006+Compound 24 treatment group; (D) is the Influenza A/Califonria/07/2009 (H1N1)pdm09+PBS control group; (E) is the Influenza A/Wisconsin/67/2005 (H3N2)+PBS control group; and (F) is the Influenza B/Florida/04/2006+PBS control group.

FIG. 31 shows graphs of cytokine and chemokine levels measured in the lungs of mice from treatment or control groups.

FIG. 32 shows graphs of serum cytokine and chemokine levels measured in treatment or control groups to observe the presence or absence of systemic inflammation in the test subjects.

FIG. 33 shows qualitative histological analysis images of lung tissue biopsies taken from mice infected with Influenza A/H1N1/PR8/1934 and treated with 1) PBS, 2) Compound 24 via IV, or 3) Compound 24 via OG.

FIG. 34 shows histological images taken of lung or kidney tissue of virus infected mice following treatment with Compound 24 (orally or intravenously) or no treatment.

FIG. 35 shows a synthesis scheme of folate-dual-hapten conjugate Compound 150.

FIG. 36 shows LC-MS, mass spectrometry, and ultraviolet spectrometry data of Compound 3′.

FIG. 37 shows LC-MS, mass spectrometry, and ultraviolet spectrometry data of Compound 5′.

FIG. 38 shows LC-MS, mass spectrometry, and ultraviolet spectrometry data of Compound 10′.

FIG. 39 shows LC-MS, mass spectrometry, and ultraviolet spectrometry data of Compound 11′.

FIG. 40 shows LC-MS, mass spectrometry, and ultraviolet spectrometry data of Compound 13′.

FIG. 41 shows LC-MS, mass spectrometry, and ultraviolet spectrometry data of Compound 14′.

FIG. 42 shows LC-MS, mass spectrometry, and ultraviolet spectrometry data of Compound 18′.

FIG. 43 shows LC-MS, mass spectrometry, and ultraviolet spectrometry data of Compound 150.

FIG. 44 shows data related to a CDC assay illustrated in a graph of folate-dual-hapten (Compound 150=FDH) concentration (nM) versus percent killing via CDC, which shows Compound 150 executed against the murine lung cancer cells (M109) when co-cultured with anti-hapten antibodies and human serum (M109_comp=folate-glucosamine competitor).

FIGS. 45A-45D shows data related to an ADCC assay illustrated in a graphs of FDH concentration (nM) versus percent killing via ADCC, which show Compound 150 executed against multiple murine and human cancer cell lines in vitro (4T1-FR=Compound 150 and 4T1-FR_comp=folate-glucosamine competitor, executed against 4T1 cells; MDA-MB-231=Compound 150 and MDA-MB-231_comp=folate-glucosamine competitor, executed against MDA-MB-231 cells; M109=Compound 150 and M109_comp=folate-glucosamine competitor, executed against M109 cells; THP-1=Compound 150 and THP-1_comp=folate-glucosamine competitor, executed against THP-1 cells (a human leukemia monocytic cell line that serves as an in vitro model for acute myeloid leukemia (AML))).

FIG. 46 shows data related to an in vivo murine lung cancer efficacy study of Compound 150 illustrated in a graph of tumor volume versus days post tumor implantation (PBS (+Abs)=control group; anti-PD1+carboplatin+paclitaxel=SOC test article; and Compound 150 test article). Abs=antibodies, in the case of the study shown in FIG. 46, a mouse anti-PD1 antibody (InVivoMAb anti-mouse PD-1 Catalog #BE0146; InVivoGen, San Diego, CA).

FIG. 47 shows data related to the in vivo lung cancer efficacy study of Compound 150 of FIG. 46, illustrated in a graph of subject percent body weight versus days post tumor implantation (PBS (+Abs)=control group; anti-PD1+carboplatin+paclitaxel=SOC test article; and Fol-dual hapten (+Abs)=Compound 150 test article). Abs=anti-PD1 antibody.

FIG. 48 shows data related to an in vivo human lung cancer efficacy study of Compound 150 illustrated in a graph of tumor volume versus days post tumor implantation (PBS (+Abs)=control group; anti-PD1+radiotherapy=SOC test article; and Compound 150 test article).

FIG. 49 shows safety data related to the in vivo human lung cancer efficacy study of Compound 150 of FIG. 48, illustrated in a graph of subject percent body weight versus days post tumor implantation (PBS (+Abs)=control group; anti-PDL1+radiotherapy=SOC test article; and Compound 150 test article).

FIG. 50 shows data related to an in vivo murine colorectal cancer efficacy study of Compound 150 illustrated in a graph of tumor volume versus days post tumor implantation (PBS (+Abs)=control group; Leucovorin+5-FU+oxaliplatin=SOC test article; and Fol-dual hapten (+Abs)=Compound 150 test article).

FIG. 51 shows safety data related to the in vivo colorectal cancer efficacy study of Compound 150 of FIG. 50, illustrated in a graph of subject percent body weight versus days post tumor implantation (PBS (+Abs)=control group; anti-PD1+carboplatin+paclitaxel=SOC test article; and Compound 150 (+Abs) test article).

FIG. 52 shows data related to an in vivo murine lung cancer combination therapy study, illustrated in a graph of tumor volume versus days post tumor implantation.

FIG. 53 shows data related to an in vivo murine lung cancer combination therapy study, illustrated in a graph of tumor volume versus days post tumor implantation.

FIG. 54 shows data related to an in vivo murine lung cancer combination therapy study, illustrated in a graph of tumor volume versus days post tumor implantation (PBS (+Abs)=control group; Leacovorin+5FU+OXH=SOC test article; and Fol-dual hapten (+Abs)=Compound 150 test article).

FIG. 55 shows data related to the in vivo human lung cancer efficacy study of Compound 150 of FIG. 54, illustrated in a graph of subject percent body weight versus days post tumor implantation (PBS (+Abs)=control group; Leacovorin+5FU+OXH=SOC test article; and Fol-dual hapten (+Abs)=Compound 150 test article).

FIG. 56 shows data related to an in vivo murine colorectal cancer combination therapy study, illustrated in a graph of tumor volume versus days post tumor implantation.

FIG. 57 shows data related to an in vivo murine colorectal cancer combination therapy study, illustrated in a graph of tumor volume versus days post tumor implantation (PBS (+Abs)=control group; Leacovorin+5FU+OXH=SOC test article; Fol-dual hapten (+Abs)=Compound 150 test article; Fol-dual hapten+sunitinib=Compound 150+sunitinib; Fol-dual hapten+FA-TLR7=Compound 150+FA-TLR7).

FIG. 58 shows data related to an in vivo murine lung cancer comparison efficacy study of Compound 150 versus anti-PD1+chemotherapy treatment, illustrated in a graph of tumor volume versus days post tumor implantation (PBS (+Abs)=control group; anti-PD1+chemo=SOC test article; and Fol-dual hapten (+Abs)=Compound 150 test article).

FIG. 59 shows data related to an in vivo murine lung cancer (cold nature) efficacy study of Compound 150 illustrated in a graph of tumor volume versus days post tumor implantation.

FIG. 60 shows data related to an in vivo murine renal cancer (Renca cell line) efficacy study, illustrated in a graph of tumor volume versus days post tumor implantation.

FIG. 61 shows graphs of comparison data of immune cell populations in Y856, LLC-1, and M109 cell lines (cold vs. hot) measured following treatment with PBS (control), Compound 150 (FDH), or SOC.

While the present disclosure is susceptible to various modifications and alternative forms, exemplary embodiments thereof are shown by way of example in the drawings and are herein described in detail.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

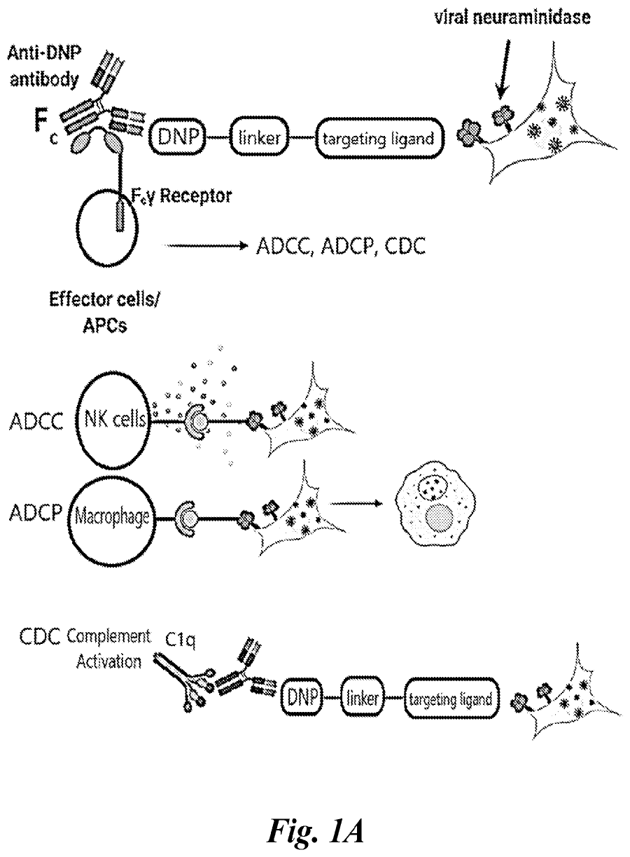

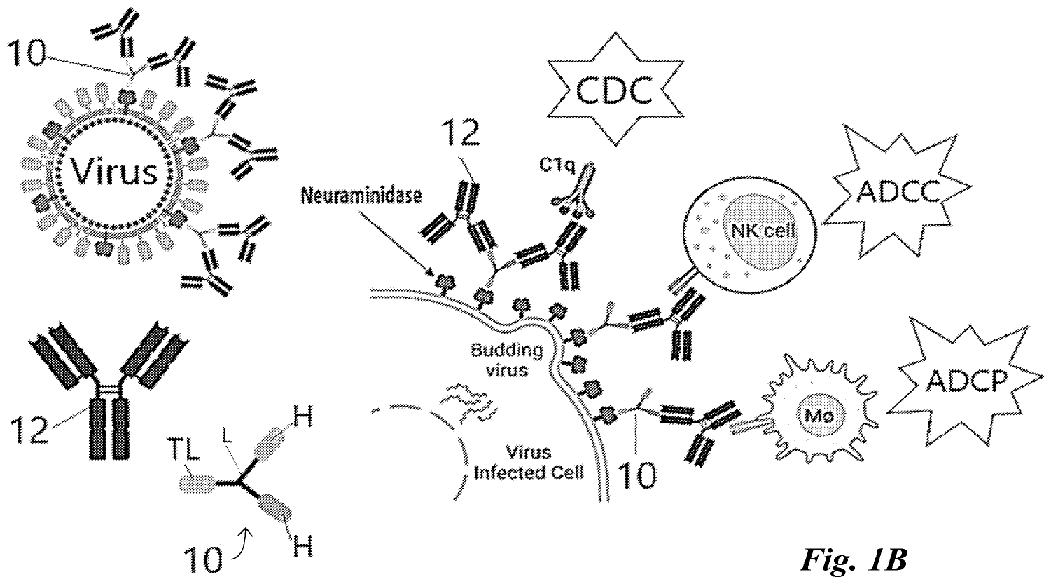

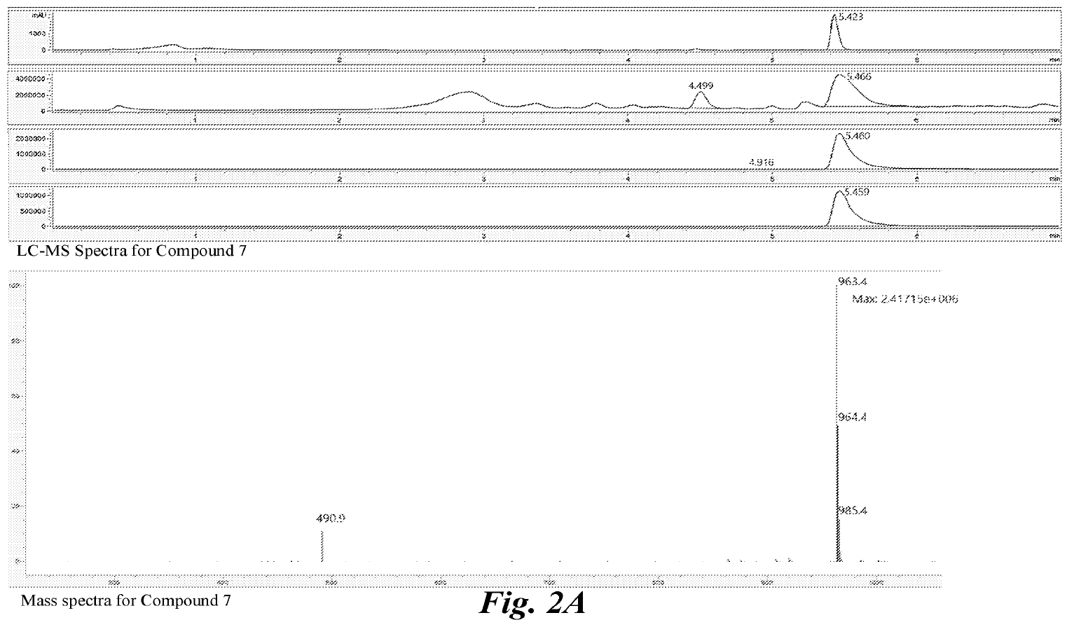

The present disclosure provides materials and methods to recruit antibodies, such as to the surface of a virus, a virus-infected cell, a cancer cell, an immune cell, or a fibroblast. See, e.g., FIG. 1A for a schematic explanation of antibody recruitment.

More specifically, a targeted therapeutic strategy with a dual mechanism of action that elicits host immune response against a target (e.g., a virus, a virus-infected cell, a cancer cell) is disclosed. The conjugates hereof can be trivalent such that they comprise a targeting ligand bound (e.g., via a linker) to at least two haptens that bind to naturally occurring antibodies (e.g., in humans). Once recruited, these anti-hapten antibodies bind and activate innate immune system against the target. Pharmaceutical compositions comprising such conjugates, and methods comprising the administration of such conjugates and compositions are also provided.

### Conjugates

The conjugates hereof can be small molecule ligand-targeted drug conjugates that combine a receptor-specific ligand with at least two haptens. Accordingly, the conjugates can be a trivalent drug that can target a desired cell via the receptor-specific ligand (such as a targeting ligand) and also bind two or more antibodies in a subject via the dual payload. The general scheme is to provide a specific targeting ligand conjugated to an effective payload of two or more haptens to treat viral infections or cancer. The targeting ligand can specifically recognize a target receptor (e.g., an envelop protein of a virus (which can be exclusively expressed on the surface of an infected cell), or a receptor that is overexpressed on a targeted cell (e.g., a folate receptor or the like on a cancer cell). In certain embodiments, one or more of the haptens are selected to activate the innate immune system of the subject (e.g., adjacent to the targeted cell) to recruit immune cells and/or otherwise leverage the subject's own immune system against the virus or cancer.

In certain embodiments, a conjugate has the formula:

TL-L-Hₙ,

or is a pharmaceutically acceptable salt thereof, wherein:
TL is a targeting ligand for a target protein on the surface of a targeted cell;
L is a linker;
H is a hapten; and
n is an integer, which is 2 or greater.

Optionally, at least two of the Hs can be bound by a different antibody when brought into contact with antibodies present in vivo, for example. In certain embodiments, n is an integer that is greater than or equal to 2 (e.g., 2, 3, 4, 5, . . . 10, etc.). n can be 2 or 3.

In many embodiments, the conjugate has the formula:

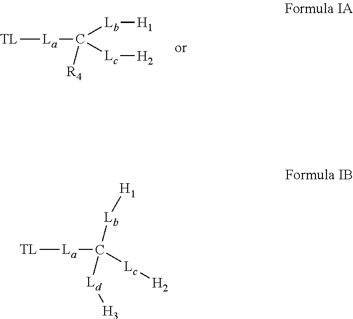

including pharmaceutical salts thereof, where TL is a targeting ligand for a target protein on the surface of a virus, a virus-infected cell, a cancer cell, an immune cell, or a fibroblast; Lₐ, L_{b}, L_{c} and L_{d} are each a linker; C is a carbon atom; and H₁, H₂, and H₃ are each a hapten.

In these and other embodiments, at least two of haptens are bound by a different antibody when brought into contact with antibodies present in vivo, for example. In these and other embodiments, H₁ and H₂ can each bind a different antibody. In these and other embodiments, H₁, H₂, and H₃ can each bind a different antibody.

Optionally, the conjugates can be complexed with an antibody through one or more of the haptens of the conjugate. When bound, each hapten is typically independently bound to different antibodies. Each antibody can be a naturally occurring autologous antibody, an exogenously administered autologous antibody, or an exogenously administered Immunoglobin G (IgG) antibody. The binding to antibodies is typically a non-covalent interaction, such as with hydrogen bonding, coulombic bonds, Ca⁺⁺ bridges, and Lifshitz-van der Waals bonds, among others. The bonding typically occurs in vivo.

Each of H or H₁, H₂, or H₃ (where applicable) can be independently selected from a different hapten. In certain embodiments, where n=2, the happens are different haptens. Where n=3 or more, two or more of the haptens can be the same. Haptens that can be used include, but are not limited to, a rhamnose fragment, an α-galactosyl moiety, a dinitrophenyl (DNP) fragment, a trinitrophenyl (TNP) fragment, a fluorescein fragment, a digoxigenin fragment, a biotin fragment, or an antigen of a virus selected from diphtheria, zoster virus, human papillomavirus (HPV), influenza virus antigen (e.g., haemagglutinin or neuraminidase), SARS-COV-2, yellow fever, respiratory syncytial virus (RSV), herpes simplex virus (HSV), varicella virus, hepatitis A (HAV) antigen (e.g., L-HbsAg, S-HbsAg, M-HbsAg, and preS), hepatitis B (HBV), hepatitis C (HCV), hepatitis D (HDV), hepatitis G (HGV), rotavirus, mumps virus, tetanus, human immunodeficiency virus (HIV) (e.g., gp120 or gp160), cytomegalovirus (CMV), vesicular stomatitis virus (e.g., glycoprotein), rubella virus, smallpox, monkeypox, poliovirus, dengue virus, and measles virus.

In some embodiments, the haptens are each independently selected from a rhamnose fragment, an α-galactosyl moiety, and a DNP fragment. In some embodiments, when n is 2, the haptens are a rhamnose fragment and a DNP fragment.

A hapten can be gp120 or gp160. The hapten can be a glycoprotein.

The targeting ligand of the conjugate can specifically recognize a targeted receptor, such as a targeted protein on a surface of a targeted cell. The targeted cell can be a virus, a virus-infected cell, a cancer cell, an immune cell, or a fibroblast.

By specifically delivering the haptens to cells recognized by the targeting ligand, the conjugates hereof can demonstrate high selectivity toward malignant cells, virus-infected cells, immune cells, and any other cell with a target protein on its surface, while also reducing collateral toxicity. To date, many cancers have been tackled by small molecule ligand-targeted drug conjugates that target overexpressing receptors on tumor cells. These overexpressing receptors include, without limitation, folate receptor (FR), prostate-specific membrane antigen (PSMA), cholecytstokinin 2 receptor (CCK2R), carbonic anhydrase IX (CA IX), and the like. The targeting ligand of the conjugate can have specificity for any of such overexpressing receptors.

The targeting ligand can also have specificity for a virus envelop protein. For example, the target protein can be an envelope protein of a virus or a viral envelope protein on the surface of a virus-infected cell.

Influenza virus, for example, is an enveloped virus. All influenza subtypes are very similar in overall structure and composition; namely, a virus particle is 80-120 nanometers in diameter and has a viral envelope containing two main types of glycoproteins wrapped around a central core. The central core contains the viral ribonucleic acid (RNA) genome and other viral proteins that package and protect this RNA. Unusually for a virus, influenza's genome is not a single piece of nucleic acid but rather seven or eight pieces of segmented negative-sense RNA, each piece of which contains either one or two genes that code for a gene product (protein). For example, the influenza A genome contains 11 genes on eight pieces of RNA that encode for 11 proteins: hemagglutinin (HA), neuraminidase (NA), nucleoprotein (NP), M1, M2, NS1, NS2 (NEP: nuclear export protein), PA, PBT (polymerase basic 1), PB1-F2, and PB2.

HA and NA are the two large glycoproteins on the outside of the viral particles. HA is a lectin that can mediate binding of the virus to target cells and entry of the viral genome into a target cell, while NA is typically involved in the release of progeny virus from infected cells by cleaving sugars that bind the mature viral particles. The target protein can be influenza NA or influenza HA.

In some embodiments of the conjugate hereof, the targeting ligand is an NA or HA inhibitor, which can be used to deliver the conjugate into a virus infected cell and/or virus replication sites (e.g., nose, throat, and lungs). This allows for killing virus infected cells prior to the progeny virus release, hindering viral replication, and/or dampening the early cytokine storm induced by viral infection. In certain embodiments, the targeting ligand is an oseltamivir fragment, a zanamivir fragment, a peramivir fragment, or a laninamivir fragment. In certain embodiments, the targeting ligand is zanamivir.

In other embodiments, the target protein is selected from respiratory syncytial virus (RSV) fusion protein F, a coronavirus spike protein, be hepatitis B virus (HBV) surface antigen or HBV core antigen, a cell-surface receptor on a cancer cell, folate receptor α, prostate-specific membrane antigen (PSMA), carbonic anhydrase 9 (CAIX), luteinizing hormone releasing receptor (LHRH), neurokinin 1 receptor (NKTR), a cell-surface receptor on a tumor-associated macrophage (TAM) or a myeloid-derived suppressor cells, folate receptor β, a cell-surface receptor on a cancer-associated fibroblast, and a fibroblast activation protein (FAP).

In certain embodiments, the targeting ligand is folate or an analog thereof, such as 5-methyltetrahydrofolate (5-MTHF). “Folate” means a folate receptor-binding molecule, including for example folic acid and analogs and derivatives of folic acid such as, without limitation, folinic acid, pteroylpolyglutamic acid, pteroyl-D-glutamic acid, and folate receptor-binding pterdines such as tetrahvdropterins, dihydrofolates, tetrahydrofolates, and their deaza and dideaza analogs.

The terms “deaza” and “dideaza” analogs refer to the art-recognized analogs having a carbon atom substituted for one or two nitrogen atoms in the naturally occurring folic acid structure, or analog or derivative thereof. For example, the deaza analogs may include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate. Other folates useful as complex forming ligands in the context of the present disclosure are the folate receptor-binding analogs pemetrexed, proguanil, pyrimethamine, trimethoprim, pralatrexate, raltitrexed, aminopterin, amethopterin (also known as methotrexate), N¹⁰-methylfolate, 2-deamino-dydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate).

Folic acid and the foregoing analogs and/or derivatives are also termed “a folate,” “the folate,” or “folates” reflecting their ability to bind to folate-receptors. As described herein, such molecules, when conjugated with exogenous molecules, are effective to enhance transmembrane transport, such as via folate-mediated endocytosis. The foregoing can be used in the folate receptor-targeting ligands described herein.

In many embodiments, the conjugate comprises a linker (L) that couples or otherwise connects each hapten to the targeting ligand. In many embodiments, the conjugate comprises linkers Lₐ, L_{b}, L_{c}. In some embodiments, the conjugate comprises linkers Lₐ, L_{b}, L_{c}, and L_{d}. Where the conjugate comprises more than one linker (e.g., Lₐ, L_{b}, L_{c}, and, optionally, L_{d}), the linkers can be the same or different. For example, and without limitation, each of Lₐ, L_{b}, L_{c}, and L_{d} could have the same structure. Alternatively, one or more of the linkers can have a different structure as compared to at least one of the other linkers. In certain embodiments, Lₐ comprises a first structure and L_{b}, L_{c}, and, when present, L_{d} comprise a second structure. In certain embodiments, L_{b}, L_{c}, and, when present, L_{d} comprise different structures from each other.

As used herein, the term “linker” includes a chain of atoms that is bio-functionally adapted to form a chemical bond with a TL and/or hapten and/or a carbon atom and connects two or more functional parts of a molecule to form a conjugate hereof Illustratively, the chain of atoms can be selected from carbon (C), nitrogen (N), oxygen (O), sulfur (S), silicon (Si), and phosphorus (P). The chain of atoms can covalently connect different functional capabilities of the conjugate, such as the targeting moiety and the haptens. The linker can comprise a wide variety of links, such as in the range from about 2 to about 100 atoms in the contiguous backbone. The linker can comprise a slow-release linker (e.g., a non-hydrolyzable linker).

In certain embodiments where the conjugate comprises two haptens, the conjugate can comprise at least three linkers—the first linker connecting the targeting moiety to a carbon atom, the second linker connects the carbon atom to a first hapten and the third linker connects the carbon atom to a second hapten as shown in Formula IA and Formula IB hapten, and the second linker connecting the targeting moiety to the second hapten. Alternatively, the conjugate can comprise a single branching linker that connects all haptens to the targeting moiety (see, e.g., Formula I). In certain embodiments, the linker is a branched linker and at least two of the haptens of the conjugate are connected to different branches of the linker. For example, the linker can comprise a backbone that comprises at least two branches (e.g., 2 branches, 3 branches, etc.) extending therefrom. The different branches can comprise the same structure or different structures as the backbone and/or the other branches. The different branches can, for example, extend from different atoms of a backbone linker or extend from the same atom (e.g., as shown in Formulae IA and IB).

In many embodiments, the linker comprises a polyethylene glycol (PEG) linker, a PEG derivative linker, a peptide, an alkyl group, a peptidoglycan, or a combination of two or more of the foregoing. In some embodiments, the PEG linker can comprise (—CH₂CH₂—O—)ₙ, where n is an integer between and including 1 and 32. In certain embodiments, n of the PEG linker is an integer between and including 1 and 16. In certain embodiments, the linker is an optionally substituted heteroalkyl.

In some embodiments, the linker is a substituted heteroalkyl comprising at least one substituent selected from the group consisting of alkyl, hydroxyl, oxo, PEG, carboxylate, and halo. “Halo” or “halogen” by itself or as part of another substituent means, unless otherwise stated, a fluorine, chlorine, bromine, or iodine atom.

In some embodiments, the linker comprises a spacer. In some embodiments, the spacer comprises a peptidoglycan or a sugar.

The linker can comprise a C₂-C₁₈ alkyl group, a peptide fragment, or a peptidoglycan fragment. As used herein, the term “fragment” means a molecule that has been modified to allow linking in the conjugate either as monovalent linking, such as in the case of a targeting ligand or a hapten, or bivalent linking, such as in the case of the linkers (L). For example, the term “zanamivir fragment” means that zanamivir has been chemically modified so as to allow it to be prepared as the targeting ligand covalently bound to a linker. With respect to zanamivir, a proton, such as from an alcohol, is missing, thus creating an open valence on the corresponding oxygen, which is bound, in turn, to the linker, L. Typically, the alcohol oxygen closest to the dihydropyran ring of zanamivir forms a bond to linker L. Similarly, hapten fragments are moieties based on the corresponding molecules which have been adapted for linking to the linker, L. With regards to the linker L, the corresponding fragments have open valencies to allow for bonding to other linker moieties, targeting ligands, and/or haptens. For example, with respect to PEG, the term (—CH₂CH₂—O—)ₙ refers to a fragment that is bound on both sides to another moiety, such as another linker component, for example. The use of the term “fragment” does not require that from a synthetic perspective; the molecule it refers to is made in the preparation of the conjugate. It is a description for moiety within the conjugate, regardless of how made.

Optionally, the conjugates can be complexed to antibodies, such as when in vivo. Such complexation can be with different antibodies per hapten.

In many embodiments of the disclosure, the conjugate has the structure of Formula I:

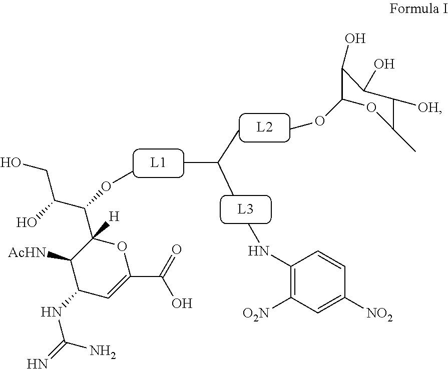 [SMILES: COC1OC(C)C(O)C(O)C1O ; CO[C@H]([C@@H]1OC(C(=O)O)=C[C@H](NC(=N)N)[C@H]1NC(C)=O)[C@H](O)CO ; CNc1ccc([N+](=O)[O-])cc1[N+](=O)[O-]]

or is a pharmaceutically acceptable salt thereof, wherein L1, L2, and L3 are each linkers. In certain embodiments, L1, L2, and L3 are each independently comprise a PEG moiety, an alkyl group, a peptide group and a peptidoglycan group. In certain embodiments, the PEG moiety (—CH₂CH₂—O—)ₙ has an n ranging between and including 1 to 16. The alkyl group can be an alkyl chain between and including 2 and about 18 carbons (e.g., 18 carbons). For example, L1, L2 and L3 can each independently comprise a C₂-C₁₈ alkyl group. L1, L2, and L3 can each independently comprise a peptide fragment or a peptidoglycan fragment.

In certain embodiments, L1 comprises an amide, which can be optionally substituted with an alkyl group, a PEG moiety, an optionally substituted or unsubstituted triazole moiety wherein, if substituted, the substitution can be a carbonylalkyl group and an amide that can optionally be substituted with an alkyl group. Further, in certain embodiments, an amide in L1 is bonded directly to the oxygen of a zanamivir fragment as set forth in Formula I, forming a carbamate moiety with the oxygen. In certain embodiments, the tertiary carbon at the intersection of L1, L2, and L3 is bonded to the nitrogen of an amide of L1. In certain embodiments, the PEG moiety is bound on one side to an amide and on the other side either to an amide or to a substituted or unsubstituted triazole moiety. The PEG moiety can be expressed as (—CH₂CH₂—O—)ₙ where n can be between and including 1 and 32, between and including 2 and 16, between and including 3 and 8, and between and including 4 and 7. In some embodiments, n is 6.

In certain embodiments, L2 comprises an amide moiety optionally substituted with an alkyl group and a PEG moiety. The PEG moiety can be expressed as (—CH₂CH₂—O—)ₙ where n can generally be between and including 1 and 32, between and including 2 and 16, between and including 3 and 8, and between and including 3 and 5. In some embodiments, n is 4.

In certain embodiments, L3 comprises an alkyl amide group, a carbonyl alkyl group, an ether alkyl group, and an amine group which can optionally be substituted with an alkyl group. In some embodiments, L3 comprises a PEG. In some embodiments, L3 comprises a PEG expressed as (—CH₂CH₂—O—)ₙ, where n can be between and including 1 and 32.

In certain embodiments, provided is a dual-hapten conjugate having the structure of Compound 24:

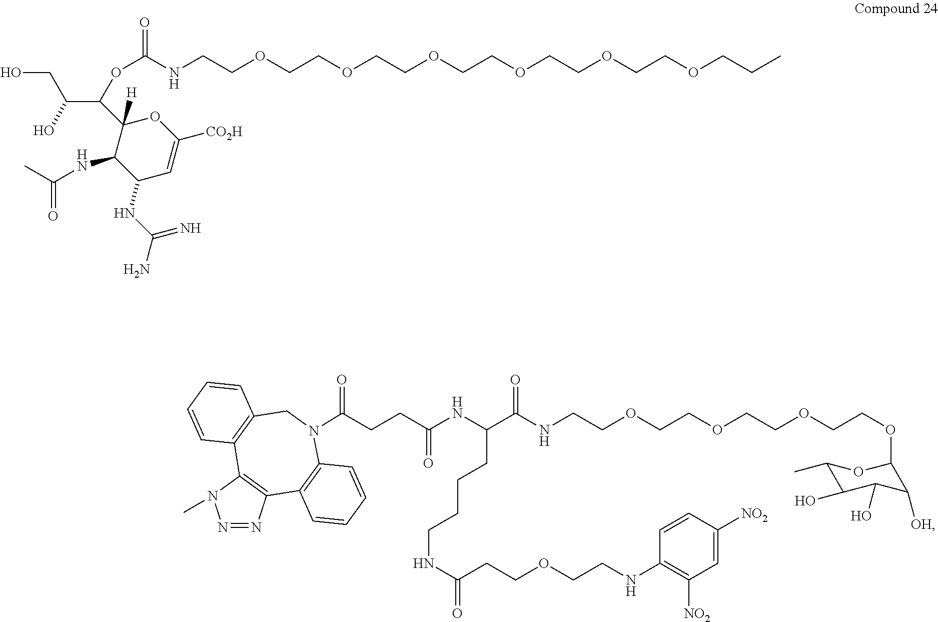

or is a pharmaceutically acceptable salt thereof.

In certain embodiments, provided is a triple hapten conjugate having the structure of Compound 100:

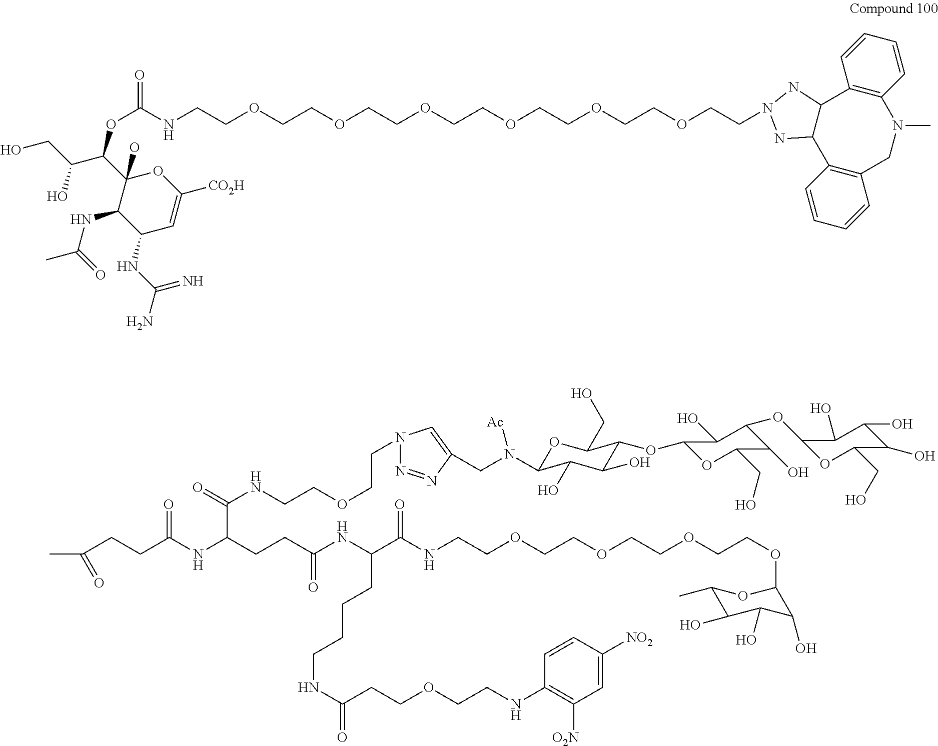

or a pharmaceutically acceptable salt thereof.

In certain embodiments, the conjugate is a dual hapten conjugate having the structure of Compound 101:

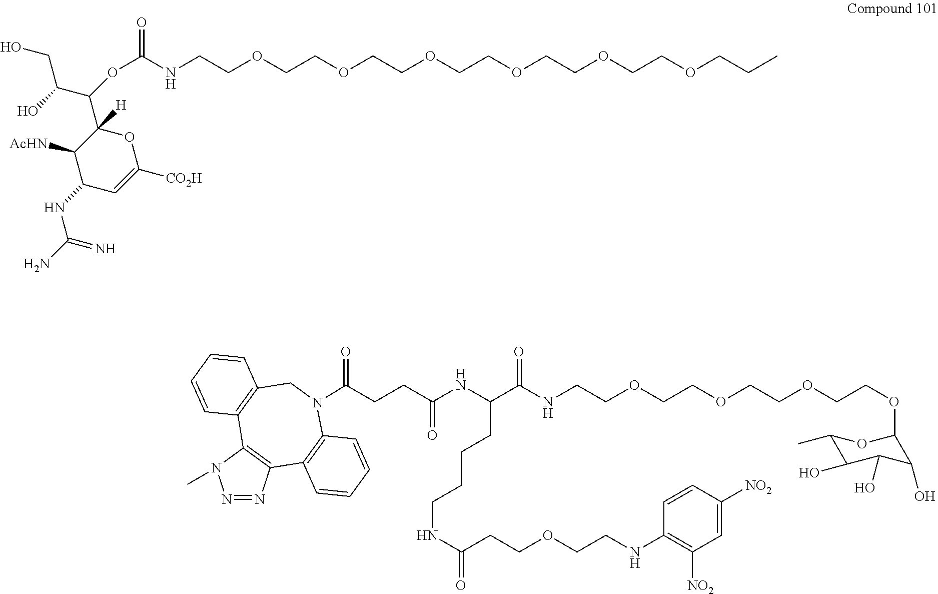

or is a pharmaceutically acceptable salt thereof, wherein Ac is acetate.

In certain embodiments, the conjugate comprises a folate targeting moiety. In certain embodiments, the conjugate is a folate-dual hapten conjugate. In certain embodiments, the conjugate is a folate-dual-hapten conjugate and has a structure of Compound 150:

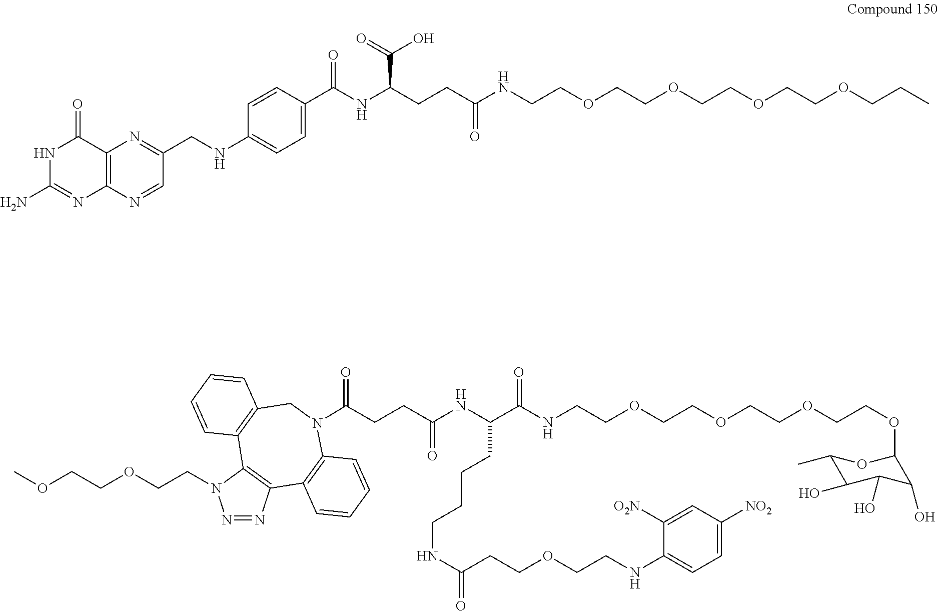

or is a pharmaceutically acceptable salt thereof.

In some embodiments, one or more of the hydroxyl (—OH) groups in the structure of Compounds 24, 100, 101, and/or 150, can be independently replaced with a thiol, a phosphate or a phosphonate ester or —OC(═O)R₁, where R₁ is an alkyl group. In such embodiments, the amine (—NH₂) group can be replaced with —OC(═O)R₂, in which R₂ is an alkyl group. In these and other embodiments, the carboxyl (—COOH) group can be replaced with —OC(═O)R₃, in which R₃ is an alkyl group. R₂ and R₃ can each be, for example, a C₁ to C₆ alkyl group.

Optionally, the conjugate hereof (e.g., Compound 24, Compound 100, Compound 101, or Compound 150) is complexed with antibodies in vivo. Such complexation can be with different antibodies per hapten.

In certain embodiments, processes for making the conjugates hereof are provided. For example, the conjugate of Compound 24 can be prepared as set forth in Example 1 and/or the conjugate of Compound 150 can be prepared as set forth in Example 19. Likewise, other conjugates hereof can be prepared in accordance with the processes of Examples 1 and/or 19 and such processes otherwise known in the art.

In certain embodiments, the conjugate is formulated as a prodrug. The term “prodrug” means a derivative of a conjugate that can hydrolyze, oxidize, or otherwise react under biological conditions (in vitro or in vivo) to provide an active compound or conjugate, particularly a multi-hapten conjugate disclosed herein. Examples of prodrugs include, but are not limited to, derivatives and metabolites of a conjugate hereof that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Formulation of the conjugate as a prodrug can incorporate protecting groups which can further slow hydrolysis of the conjugate in vivo. This can be beneficial where the prodrug is administered to reprogram immune cells in a targeted area and/or to slow cytokine activations in a targeted area.

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Salts of the compounds described herein, such as pharmaceutically acceptable salts, can be prepared by various methods, such as inclusion of an acid in the mobile phases during chromatography purification, or stirring of the products after chromatography purification, with a solution of an acid.

The conjugates hereof can be “deuterated,” meaning one or more hydrogen atoms can be replaced with deuterium. As deuterium and hydrogen have nearly the same physical properties, deuterium substitution is the smallest structural change that can be made. Deuteration is well known to those of ordinary skill in the art.

The conjugates, in some embodiments, can contain one or more asymmetric centers and thus give rise to enantiomers, diastereomers, and other stereoisomeric forms that are defined, in terms of absolute stereochemistry, as (R)- or (S)-. In certain embodiments, the conjugate is of R-configuration. In certain embodiments, the conjugate is of S-configuration. Unless stated otherwise, it is intended that all stereoisomeric forms of the conjugates are contemplated. When the conjugates contain alkene double bonds, and unless specified otherwise, it is intended that both E and Z geometric isomers (e.g., cis or trans) and/or optical isomers are included. In certain embodiments, for example, D and A of a conjugate are arranged in a relative cis orientation. In certain embodiments, D and A of a conjugate are arranged in a relative trans orientation. Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Further, in each of the foregoing and following embodiments, it is to be understood that the formulae include and represent not only all pharmaceutically acceptable salts of the conjugates, but also include any and all hydrates and/or solvates of the conjugate formulae or salts thereof. Indeed, hydrates, solvates, and N-oxides of the conjugates are also contemplated. The term “solvate” means a conjugate, or a salt thereof, that further includes a stoichiometric or non-stoichiometric amount of solvent bound by non-covalent intermolecular forces. Where the solvent is water, the solvate is a hydrate.

It will be appreciated that certain functional groups, such as the hydroxy, amino, and like groups, form complexes and/or coordination compounds with water and/or various solvents, in the various physical forms of the conjugates. Accordingly, the above formulae are to be understood to include and represent those various hydrates and/or solvates.

In each of the foregoing and following embodiments, it is also to be understood that the formulae include and represent any and all crystalline forms, partially crystalline forms, and non-crystalline and/or amorphous forms of the conjugates.

### Pharmaceutical Compositions

Pharmaceutical compositions are further provided. The pharmaceutical composition can comprise any of the conjugates described herein (e.g., a conjugate of Formula I) or the pharmaceutically acceptable salts thereof, and one or more pharmaceutically acceptable excipients or carriers. The term “composition” generally refers to any product comprising more than one ingredient, including the conjugate. The compositions can be prepared from isolated conjugates or from salts, solutions, hydrates, solvates, and other forms of the conjugates.

In certain embodiments, the pharmaceutical composition comprises a plurality of conjugates (e.g., two or more) and a pharmaceutically acceptable excipient. In certain embodiments, a pharmaceutical composition further comprises at least one additional pharmaceutically active agent. The at least one additional pharmaceutically active agent can be an agent useful in the treatment of viral infection, fibrosis, or cancer.

Pharmaceutical compositions can be prepared by combining one or more conjugates with a pharmaceutically acceptable excipient and, optionally, one or more additional pharmaceutically active agents in accordance with methods known in the art and described herein below.

The pharmaceutical compositions hereof can comprise one or more pharmaceutically acceptable carriers, adjuvants, diluents, excipients, and/or vehicles (e.g., conventional nontoxic pharmaceutically acceptable carriers, adjuvants, and vehicles), and combinations thereof. Any pharmaceutically acceptable carriers, diluents, and excipients as known in the art can be used. Examples include, but are not limited to, an excipient, a color additive, a preservative, and a stabilizer. More specific examples include crystal cellulose, calcium carmellose, sodium carmellose, hydropropylcellulose, hydroxypropylmethylcellulose, ethylcellulose, and magnesium stearate.

Solutions of the conjugate or pharmaceutical composition can be aqueous, optionally mixed with a nontoxic surfactant, and/or can contain carriers or excipients, such as salts, carbohydrates and buffering agents (preferably at a pH of from 3 to 9), but, for some applications, they can be more suitably formulated as a sterile non-aqueous solution or as a dried form to be used in conjunction with a suitable vehicle, such as sterile, pyrogen-free water, or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The conjugates can be formulated as pharmaceutical compositions and administered to a mammalian host, such as a human patient, in a variety of forms adapted to the chosen route of administration. The pharmaceutical compositions can be formulated, e.g., for a given route of administration, and manufactured in accordance with methods in the art and described, for example, in Remington, The Science and Practice of Pharmacy, 22^{nd} edition (2012). The composition can be an infusion or an injectable composition, such as a composition that can be injected subcutaneously or intravenously.

The pharmaceutical composition can be administered to a mammalian host, such as a human patient, in a variety of forms adapted to the chosen route of administration. In certain embodiments, the pharmaceutical composition is formulated to be administered subcutaneously. In certain embodiments, the pharmaceutical composition is formulated to be administered orally. In certain embodiments, the pharmaceutical composition is formulated to be administered intramuscularly, intravenously, intraarterially, intraperitoneally, or as any other art-recognized route of parenteral administration.

The pharmaceutical composition can be systemically administered in combination with a pharmaceutically acceptable vehicle. The percentages of the components of the compositions and preparations can vary and can be between about 1 to about 99% weight of the active ingredient(s) (e.g., the compound or conjugate) and a binder, an excipient, a disintegrating agent, a lubricant, and/or a sweetening agent (as are known in the art). The amount of active conjugate in such therapeutically useful compositions is such that an effective dosage level can be obtained (e.g., in the serum or targeted tissue or cell).

Illustrative means of parenteral administration include needle (including microneedle) injectors, needle-free injectors and infusion techniques, as well as any other means of parenteral administration recognized in the art. Parenteral formulations are typically aqueous solutions, which can contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, can readily be accomplished using standard pharmaceutical techniques well-known to those skilled in the art.

The pharmaceutical dosage forms suitable for administration can include sterile aqueous solutions or dispersions or sterile powders comprising the active ingredients that are adapted for the extemporaneous preparation of sterile injectable or infusible solutions or dispersions, optionally encapsulated in liposomes, nanocrystals, or polymeric nanoparticles. In all cases, the ultimate dosage form should be sterile, fluid, and stable under the conditions of manufacture and storage. The liquid carrier or vehicle can be a solvent or liquid dispersion medium comprising, for example and without limitation, water, electrolytes, sugars, ethanol, a polyol (e.g., glycerol, propylene glycol, liquid polyethylene glycols, and the like), vegetable oils, nontoxic glyceryl esters, and/or suitable mixtures thereof. In at least one embodiment, the desired fluidity can be maintained by the formation of liposomes, by the maintenance of the required particle size in the case of dispersions or by the use of surfactants.

Sterile injectable solutions can be prepared by incorporating the pharmaceutical compositions in the required amount of the appropriate solvent with one or more of the other ingredients set forth above, as required, followed by filter sterilization. In the case of sterile powders for the preparation of sterile injectable solutions, vacuum-drying and freeze-drying techniques can be employed, which can yield a powder of the active ingredient plus any additional desired ingredient present in the previously sterile-filtered solutions.

### Methods

Further in view of the above, a method of treating a viral infection in a subject is also provided. The method can comprise administering, such as orally (e.g., from about 0.01 μm/kg to about 10 m/kg is of the conjugate) or intravenously, to the subject an effective amount of the above-described conjugate, prodrug, or composition and a pharmaceutically acceptable excipient. In certain embodiments, an “effective amount” is an amount effective to treat a viral infection, such as influenza. The method can further comprise administering autologous antibodies or allogeneic Immunoglobulin G (IgG) antibodies.

The viral infection can be influenza. The viral infection can be Influenza A. The viral infection can be Influenza B. The method can elicit an immune response leading to clearance of an antibody (Ab)-coated virus or an Ab-coated-virally infected cell via Ab-dependent cellular phagocytosis (ADCP), Ab-dependent cellular cytotoxicity (ADCC), and/or complement-dependent cytotoxicity (CDC).

Still further in view of the above, a method of treating cancer in a subject is also provided. The method comprises administering to the subject an effective amount of an above-described conjugate, prodrug, or composition and a pharmaceutically acceptable excipient. The method can further comprise administering autologous antibodies, allogeneic IgG antibodies, or IVIG.

The cancer can be lung cancer, bone cancer, pancreatic cancer, skin cancer, cancer of the head, cancer of the neck, cutaneous melanoma, intraocular melanoma, uterine cancer, ovarian cancer, endometrial cancer, epithelial cancer, leiomyosarcoma, rectal cancer, stomach cancer, colon cancer, breast cancer, triple-negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, bladder cancer, gastric cancer, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, or adenocarcinoma of the gastroesophageal junction. The cancer can be lung cancer, triple negative breast cancer, colon cancer, gastric cancer, bladder cancer, prostate cancer, or pancreatic cancer.

In certain embodiments, the cancer is a hot cancer. Cancers which are refractory to treatment with a checkpoint inhibitor and typically have low immune infiltration are often referred to as “cold” cancers. Conversely, cancers or tumors which respond to treatment are often referred to as “hot” cancers, and typically have receptors on the cancer cell surface and are likely to trigger a strong immune response in the subject. The hot cancer can be renal cancer. The hot cancer can be lung cancer. The hot cancer can be colorectal cancer.

Combination therapies are also provided. In certain embodiments of a method for treating a viral infection or a cancer, the method further comprises administering a second therapy to the subject. In the case of methods for treating cancer, the second therapy can comprise administering chemotherapy (e.g., an effective amount of chemotherapy). The second therapy can comprise administering (e.g., an effective amount) of sunitinib. The second therapy can comprise administering (e.g., an effective amount) of a PD-1 inhibitor. The second therapy can comprise administering (e.g., an effective amount) of a PDL-1 inhibitor.

The second therapy can comprise administration (e.g., an effective amount) of a folate-toll-like receptor 7 (FA-TLR7) agonist conjugate. As FA-TLR7 agonists can reprogram activated macrophages in the tumor microenvironment (TME) by converting the pro-tumor macrophages into anti-tumor macrophages, this can act synergistically with Compound 150 to further reduce tumor growth.

In certain embodiments, the second therapy comprises one or more of a chemotherapy, radiation therapy, sunitinib, a PD-1 inhibitor, or a PDL-1 inhibitor. In certain embodiments, the method further comprises imaging the subject (e.g., using known imaging modalities) to capture an image of the cancer (e.g., a cancerous tumor).

A method of treating fibrosis in a subject is also provided. The method comprises administering to the subject an effective amount of an above-described conjugate, prodrug, or composition and a pharmaceutically acceptable excipient. The method can further comprise administering autologous antibodies, allogeneic IgG antibodies, or IVIG.

Methods are also provided for activating an immune response in a subject (e.g., in a targeted area of a subject such as a TME). In certain embodiments, a method for activating an immune response in a subject comprises administering to the subject an effective amount of the above-described conjugate, prodrug, or composition and a pharmaceutically acceptable excipient. The immune response can be an innate immune response. The immune response can be activated in a targeted area of the subject such as a TME or a location of a virus replication site (e.g., proximal to one or more virus infected cells). Where a prodrug is administered, such prodrug can further slow hydrolysis of the underlying conjugate and/or slow cytokine activation in the targeted area of the subject.

The method for activating an immune response can further comprise administering to the subject autologous antibodies, allogeneic IgG antibodies, or human IVIG.

Administration of the effective amount of the conjugate or composition can induce reprogramming of M2-type macrophages to M1-type macrophages in the targeted area. Generally, and without any intended limitation, the novel conjugates, compositions, and methods hereof can target the innate immune system of a subject and reprogram the polarization of a macrophage from M2-type to M1-type in favor of the proinflammatory properties of the M1-type phenotype. For example, in at least one exemplary embodiment, such conjugates and compositions comprise a targeting moiety to target a folate receptor (e.g., FRO), such as a folate receptor binding ligand, or an analog, functional fragment, derivative, or a radical thereof (e.g., a pteroyl amino acid), coupled with two or more haptens via a linker. Such embodiments utilize the limited expression of the folate receptor to localize systemically administered conjugates and compositions directly to the folate-expressing cells (e.g., those of cancerous tissue) such that the haptens components can then bind desired antibodies, which recruit immune cells to the targeted site and reprogram activated myeloid cells (e.g., M2-like macrophages) into a proinflammatory M1 polarization. This targeting design advantageously prevents the systemic activation of the immune system (i.e. reduces systemic exposure to such conjugate/composition) and, thus, avoids toxicity, while facilitating activation of the subject's own immune system at or adjacent to the targeted site. As noted above, in certain embodiments, the conjugates or composition hereof can be administered with a second therapy comprising administering an effective amount of a FA-TLR7 agonist, which can function synergistically with the conjugates/compositions hereof by further facilitating the conversion of activated macrophages into anti-tumor (M1-like) macrophages.

The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

An “effective amount” and “an amount effective to treat” refer to any amount that is sufficient to achieve a desired biological effect. Combined with the teachings provided herein, by choosing among the various active conjugates and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the infection, cancer, or other condition being treated, the particular conjugate or composition being administered, concurrently or sequential treatments being administered, the size of the subject, or the severity of the disease or condition. One of ordinary skill in the art can empirically determine the effective amount of a particular compound and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the conjugate, such as twice daily doses.

An “effective amount” (or “effective amount to treat (therapeutically)”) of a conjugate (used interchangeably herein with “compound”) with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

For any conjugate, a therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use, any compound can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 20-2,000 mg of compound per kg body weight of the subject per day. The compounds can be administered in a dose equal or equivalent to 50-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. Where a precursor or prodrug of a compound is to be administered, it is administered in an amount that is equivalent to, i.e., sufficient to deliver, the above-stated amounts of the compound.

The formulations of the compounds can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments can be used to optimize the dose and dosing frequency for any particular compound.

The conjugates can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The conjugates can be administered at a dosage in the range from about 0.2 milligram/kg/day to greater than about 100 mg/kg/day. For example, the dosage can be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The conjugates can be administered at a dosage in the range from about 0.25 milligram/kg/day to about 25 mg/kg/day. For example, the dosage can be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The conjugate or a precursor thereof can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The conjugate or a precursor thereof can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the conjugate can be administered to a subject by any mode that delivers the conjugate to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For oral administration, the conjugates can be formulated readily by combining the active conjugate(s) with pharmaceutically acceptable excipients well-known in the art. Such excipients enable the conjugates to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions, or can be administered without any excipients.

Also contemplated are oral dosage forms of the conjugates. The conjugates can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., Preparation and properties of adducts of streptokinase and streptokinase-plasmin complex with polyethylene glycol and pluronic polyol F38, J Appl Biochem 4: 185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is typically utilized. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

The conjugate can be included in the formulation as fine multi-particulates in the form of granules or pellets of particle size about 1 mm. The formulation of the material for capsule administration could also be as a powder, lightly compressed plugs or even as tablets. Therapeutic agent could be prepared by compression.

Colorants and flavoring agents may all be included. For example, the compound can be formulated (such as by liposome or microsphere encapsulation) and then further contained within an edible product, such as a refrigerated beverage containing colorants and flavoring agents.

One may dilute or increase the volume of the compound with an inert material. These diluents can include carbohydrates, especially mannitol, a-lactose, anhydrous lactose, cellulose, sucrose, modified dextrans and starch. Certain inorganic salts also can be used as fillers including calcium triphosphate, magnesium carbonate and sodium chloride. Some commercially available diluents are Fast-Flo, Emdex, STA-Rx 1500, Emcompress and Avicell.

Disintegrants can be included in the formulation of therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders and these can include powdered gums such as agar, Karaya or tragacanth. Alginic acid and its sodium salt are also useful as disintegrants.

Binders can be used to hold the compound together to form a hard tablet and include materials from natural products such as acacia, tragacanth, starch and gelatin. Others include methyl cellulose (MC), ethyl cellulose (EC) and carboxymethyl cellulose (CMC). PVP and HPMC can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents which can be used include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei & Garren, Pulmonary delivery of peptide drugs: Effect of particle size on bioavailability of leuprolide acetate in healthy male volunteers, J Pharmaceutical Research 7: 565-569 (1990); Adjei et al., Bioavailability of leuprolide following intratracheal administration to beagle dogs, International J Pharmaceutics 63:135-144 (1990) (leuprolide acetate); Braquet et al., Effect of endothelin-1 on blood pressure and bronchopulmonary system of the guinea pig, J Cardiovascular Pharmacology 13 (suppl. 5):143-146 (1989) (endothelin-1); Hubbard et al., Annals of Internal Medicine 3: 206-212 (1989) (al-antitrypsin); Smith et al., Pulmonary deposition and clearance of aerosolized alpha-1-proteinase inhibitor administered to dogs and to sheep, J Clinical Investigation 84: 1145-1146 (1989) (a-1-proteinase); Oswein et al., Aerosolization of Proteins, Proceedings of Symposium on Respiratory Drug Delivery II, Keystone, Colorado, March, 1990 (recombinant hepatocyte growth hormone); Debs et al., Lung-specific delivery of cytokines induces sustained pulmonary and systemic immunomodulation in rats, J Immunology 140: 3482-3488 (1988) (interferon-gamma and tumor necrosis factor alpha) and U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same).

Contemplated for use are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The conjugates, when it is desirable to deliver them systemically, can be formulated for parenteral administration by injection, e.g., by bolus injection or continuous infusion.

Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran.

Optionally, the suspension can also contain suitable stabilizers or agents which increase the solubility of the compounds to allow for the preparation of highly concentrated solutions.

Alternatively, the active conjugates can be in powder form for constitution with a suitable vehicle, e.g., sterile pyrogen-free water, before use.

The conjugates can also be formulated in rectal or vaginal compositions such as suppositories or retention enemas, e.g., containing conventional suppository bases such as cocoa butter or other glycerides.

In addition to the formulations described above, a conjugate can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase or excipients. Examples of such excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, New methods of drug delivery, Science 249(4976): 1527-1533 (1990).

The conjugate and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions contain an effective amount of a compound as described herein and optionally one or more other therapeutic agents included in a pharmaceutically acceptable excipient. The term “pharmaceutically acceptable excipient” means one or more compatible solid or liquid fillers, diluents or encapsulating substances which are suitable for administration to a human or other vertebrate animal. The term “excipient” denotes an organic or inorganic ingredient, natural or synthetic, with which the active ingredient is combined to facilitate the application. The components of the pharmaceutical compositions also can be commingled with the compounds, and with each other, in a manner such that there is no interaction which would substantially impair the desired pharmaceutical efficiency.

Therapeutic agent(s), including specifically, but not limited to, a compound, can be provided in particles. “Particles” as used herein means nanoparticles or microparticles (or in some instances larger particles) that can consist in whole or in part of the compound or the other therapeutic agent(s) as described herein. The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Bioerodible hydrogels based on photopolymerized poly(ethylene glycol)-co-poly(.alpha.-hydroxy acid) diacrylate macromers, Macromolecules 26(4): 581-587 (1993), the teachings of which are specifically incorporated by reference herein. These include polyhyaluronic acids, casein, gelatin, glutin, polyanhydrases, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Conjugate(s) can be contained in controlled-release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

### Certain Definitions

The term “about” as used herein can allow for a degree of variability in a value or range, for example, within 10%, within 5%, or within 1% of a stated value or of a stated limit of a range.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation.

An “alkyl group” is a saturated, partially saturated, or unsaturated straight chain or branched non-cyclic hydrocarbon having from 1 to 10 carbon atoms (C₁-C₁₀ alkyl), from 1 to 8 carbons (C₁-C₈ alkyl), from 1 to 6 (C₁-C₆ alkyl), 1 to 4 (C₁-C₄ alkyl), 1 to 3 (C₁-C₃ alkyl), or 2 to 6 (C₂-C₆ alkyl) carbon atoms. In some embodiments, the alkyl group has monovalency. Examples of alkyl groups with monovalency include —CH₃, —CH₂CH₃, and the like. Monovalent alkyls may be found on substitutions in the chain of linker, L, for example. In some embodiments, the alkyl group has bivalency, such as when found in the chain of the linker, L. Examples of alkyl groups with bivalency include, but are not limited to, —CH₂—, —CH₂CH₂—, and the like. In some embodiments, the alkyl group is a saturated alkyl group. In some embodiments, an alkyl group is an unsaturated alkyl group, also termed an alkenyl group or an alkynyl group.

The term “heteroalkyl” by itself or in combination with another term means, unless otherwise stated, a stable straight or branched chain, or combination(s) thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quarternized. The heteroatom(s) 0, N, P, S, and Si may be placed at any interior position of the heteroalkyl group or at the position at which the alkyl group is attached to the remainder of the molecule. Examples include, without limitation, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃. —CH₂—CH₂—S(O)₂—CH₃, —CH₂═CHO—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Any use of section headings and subheadings is solely for ease of reference and is not intended to limit any disclosure made in one section to that section only; rather, any disclosure made under one section heading or subheading is intended to constitute a disclosure under each and every other section heading or subheading.

Various modifications and variations of the described compositions, methods, and uses of the technology will be apparent to those skilled in the art without departing from the scope and spirit of the technology as described. Although the technology has been described in connection with specific exemplary embodiments, the invention as claimed should not be unduly limited to such specific embodiments. Indeed, various modifications of the described modes for carrying out the invention that are obvious to those skilled in the art are intended to be within the scope of the following claims.

The terms and expressions, which have been employed, are used as terms of description and not of limitation. In this regard, where certain terms are defined and are described or discussed elsewhere, the definitions and all descriptions and discussions are intended to be attributed to such terms. There also is no intention in the use of such terms and expressions of excluding any equivalents of the features shown and described or portions thereof.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

### EMBODIMENTS

The following embodiments, represented as clauses, are not meant to be limiting but further describe the inventions of the disclosure.

Clause A. A conjugate having the formula:

TL-L-Hₙ

or a pharmaceutically acceptable salt thereof, wherein:
TL is a targeting ligand for a target protein on the surface of a virus, a virus-infected cell, a cancer cell, an immune cell, or a fibroblast;
L is a linker;
H is a hapten; and
n is an integer of 2-3; and optionally, wherein at least two of the Hs can each bind a different antibody when brought into contact therewith.

Clause B. The conjugate of clause A, wherein at least two of the Hs are each bound by an antibody.

Clause C. The conjugate of clauses A or B, wherein each H is bound by a different antibody.

Clause D. The conjugate of any one of clauses A-C, wherein each H is independently selected from a rhamnose fragment, an α-galactosyl moiety, a dinitrophenyl fragment, a trinitrophenyl fragment, or a combination thereof.

Clause E. The conjugate of clause A, wherein n is 2.

Clause F. The conjugate of clause A, wherein n is 3.

Clause G. The conjugate of any one of clauses A-C, E, or F, wherein each H is independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

Clause H. The conjugate of any one of clauses A-C, wherein n is 2, a first H is a DNP fragment, and a second H is a rhamnose fragment.

Clause I. The conjugate of clause A, wherein at least one H is an influenza virus antigen selected from haemagglutinin and neuraminidase.

Clause J. The conjugate of clause A, wherein at least one H is a hepatitis antigen selected from L-HBsAg, S-HBsAg, M-HBsAg, and preS.

Clause K. The conjugate of clause A, wherein at least one H is gp120 or gp160.

Clause L. The conjugate of clause A, wherein at least one H is a glycoprotein.

Clause M. The conjugate of any one of clauses A-C, E, F, or I-L, wherein the target protein is an envelope protein of a virus or a viral envelope protein on the surface of a virus-infected cell.

Clause N. The conjugate of any one of clauses A-C, E, F, or I-L, wherein the target protein is influenza neuraminidase or influenza hemagglutinin.

Clause O. The conjugate of any one of clauses A-C, E, F, or I-L, wherein the target protein is a respiratory syncytial virus fusion protein F.

Clause P. The conjugate of clauses A-C, E, F, or I-L, wherein the target protein is coronavirus spike protein.

Clause Q. The conjugate of clauses A-C, E, F, or I-L, wherein the target protein is hepatitis B virus surface antigen or HBV core antigen.

Clause R. The conjugate of clauses A-C, E, F, or I-L, wherein the target protein is a cell-surface receptor on a cancer cell.

Clause S. The conjugate of clause A, wherein the target protein is folate receptor.

Clause T. The conjugate of clause S, wherein the target protein is folate receptor α or folate receptor β.

Clause U. The conjugate of any one of clauses A-C, E, F, or I-L, wherein the target protein is a prostate-specific membrane antigen.

Clause V. The conjugate of any one of clauses A-C, E, F, or I-L, wherein the target protein is carbonic anhydrase 9.

Clause W. The conjugate of any one of clauses A-C, E, F, or I-L, wherein the target protein is luteinizing hormone releasing hormone receptor.

Clause X. The conjugate of any one of clauses A-C, E, F, or I-L, wherein the target protein is a neurokinin 1 receptor.

Clause Y. The conjugate of any one of clauses A-C, E, F, or I-L, wherein the target protein is a cell-surface receptor on a tumor-associated macrophage.

Clause Z. The conjugate of any one of clauses A-C, E, F, or I-L, wherein the target protein is a cell-surface receptor on a myeloid-derived suppressor cells.

Clause AA. The conjugate of any one of clauses A-C, E, F, or I-L, wherein the target protein is a cell-surface receptor on a cancer-associated fibroblast.

Clause BB. The conjugate of any one of clauses A-C, E, F, or I-L, wherein the target protein is a fibroblast activation protein.

Clause CC. The conjugate of any one of clauses A-C, E, F, or I-L, wherein the targeting ligand is a neuraminidase inhibitor.

Clause DD. The conjugate of any one of clauses A-C, E, F, or I-L, wherein the targeting ligand is an oseltamivir fragment, a zanamivir fragment, a peramivir fragment, or a laninamivir fragment.

Clause EE. The conjugate of clause A, wherein the targeting ligand is a zanamivir fragment.

Clause FF. The conjugate of any one of clauses A-C, E, F, I-L, S or T, wherein the targeting ligand is a folic acid fragment or an analog thereof.

Clause GG. The conjugate of any one of clauses A-C, E, F, I-L, S or T, wherein the targeting ligand is 5-methyltetrahydrofolate.

Clause HH. The conjugate of any one of clauses A-C, E, F, I-L, S, T, or EE, wherein L comprises (—CH₂CH₂—O—)ₙ, where n is an integer between and including 1 and 32, a peptide, a peptidoglycan, or a combination of two or more of the foregoing.

Clause II. The conjugate of any one of clauses A-C, E, F, I-L, S, T, or EE, wherein L is a branched linker and at least two of the haptens are connected to different branches of the linker, wherein the different branches optionally extend from different atoms of the linker.

Clause JJ. The conjugate of clause A, wherein the targeting ligand is a folic acid fragment or a derivative thereof, at least a first H comprises a rhamnose fragment, and at least a second H comprises a dinitrophenyl fragment.

Clause KK. The conjugate of any one of clauses A-JJ formulated as a prodrug.

Clause LL. A conjugate having the formula

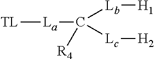

or pharmaceutically acceptable salt thereof, wherein:
TL is a targeting ligand for a target protein on the surface of a virus, a virus-infected cell, a cancer cell, an immune cell, or a fibroblast;
Lₐ. L_{b}, and L, are each a linker, which can be the same or different;
C is a carbon atom;
R₄ is selected from a hydrogen, C₁-C₅ alkyl, C₁-C₅ alkenyl, or C₁-C₅ alkynl group;
H₁ and H₂ are each a hapten; and
optionally, wherein H₁ and H₂ each can bind a different antibody.

Clause MM. A conjugate having the formula

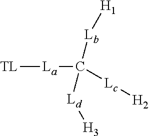

and pharmaceutically acceptable salts thereof, wherein:
TL is a targeting ligand for a target protein on the surface of a virus, a virus-infected cell, a cancer cell, an immune cell, or a fibroblast;
Lₐ, L_{b}, L_{c} and L_{d} are each a linker, which can be the same or different;
C is a carbon atom;
H₁, H₂, and H₃ are each a hapten; and
optionally, wherein each H₁, H₂ and H₃ can each bind a different antibody.

Clause NN. The conjugate of clause MM, wherein H₁ and H₂ are each bound by an antibody.

Clause OO. The conjugate of clause MM, wherein H₁, H₂, and H₃ are each bound by an antibody.

Clause PP. The conjugate of clause LL or NN, wherein H₁ and H₂ are each independently selected from a rhamnose fragment, an α-galactosyl moiety, a dinitrophenyl fragment, a trinitrophenyl fragment, or a combination thereof.

Clause QQ. The conjugate of clause MM or PP, wherein H₁, H₂ and H₃ are each independently selected from a rhamnose fragment, an α-galactosyl moiety, a dinitrophenyl fragment, a trinitrophenyl fragment, or a combination thereof.

Clause RR. The conjugate of clause LL or NN, wherein H₁ or H₂ are each independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

Clause SS. The conjugate of clause LL or 00, wherein H₁, H₂ or H₃ are each independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

Clause TT. The conjugate of clause LL or 00, wherein H₁ is a DNP fragment, and H₂ is a rhamnose fragment.

Clause UU. The conjugate of clause LL or MM, wherein at least one hapten is an influenza virus antigen selected from haemagglutinin and neuraminidase.

Clause VV. The conjugate of clause LL or MM, wherein at least one hapten is a hepatitis antigen selected from L-HBsAg, S-HBsAg, M-HBsAg, and preS.

Clause WW. The conjugate of clause LL or MM, wherein at least one hapten is gp120 or gp160.

Clause XX. The conjugate of clause LL or MM, wherein at least one hapten is a glycoprotein.

Clause YY. The conjugate of any one of clauses LL-OO, wherein the target protein is an envelope protein of a virus or a viral envelope protein on the surface of a virus-infected cell.

Clause ZZ. The conjugate of any one of clauses LL-OO, wherein the target protein is influenza neuraminidase or influenza hemagglutinin.

Clause AAA. The conjugate of any one of clauses LL-OO, wherein the target protein is a respiratory syncytial virus fusion protein F.

Clause BBB. The conjugate of any one of clauses LL-OO, wherein the target protein is coronavirus spike protein.

Clause CCC. The conjugate of any one of clauses LL-OO, wherein the target protein is hepatitis B virus surface antigen or HBV core antigen.

Clause DDD. The conjugate of any one of clauses LL-OO, wherein the target protein is a cell-surface receptor on a cancer cell.

Clause EEE. The conjugate of any one of clauses LL-OO, wherein the target protein is folate receptor.

Clause FFF. The conjugate of any one of clauses LL-OO, wherein the target protein is folate receptor α or folate receptor β.

Clause GGG. The conjugate of any one of clauses LL-OO, wherein the target protein is a prostate-specific membrane antigen.

Clause HHH. The conjugate of any one of clauses LL-OO, wherein the target protein is carbonic anhydrase 9.

Clause III. The conjugate of any one of clauses LL-OO, wherein the target protein is luteinizing hormone releasing hormone receptor.

Clause JJJ. The conjugate of any one of clauses LL-OO, wherein the target protein is a neurokinin 1 receptor.

Clause KKK. The conjugate of any one of clauses LL-OO, wherein the target protein is a cell-surface receptor on a tumor-associated macrophage.

Clause LLL. The conjugate of any one of clauses LL-OO, wherein the target protein is a cell-surface receptor on a myeloid-derived suppressor cells.

Clause MMM. The conjugate of any one of clauses LL-OO, wherein the target protein is a cell-surface receptor on a cancer-associated fibroblast.

Clause NNN. The conjugate of any one of clauses LL-OO, wherein the target protein is a fibroblast activation protein.

Clause OOO. The conjugate of any one of clauses LL-OO, wherein the targeting ligand is a neuraminidase inhibitor.

Clause PPP. The conjugate of any one of clauses LL-OO, wherein the targeting ligand is an oseltamivir fragment, a zanamivir fragment, a peramivir fragment, or a laninamivir fragment.

Clause QQQ. The conjugate of any one of clauses LL-OO, wherein the targeting ligand is a zanamivir fragment.

Clause RRR. The conjugate of any one of clauses LL-OO, wherein the targeting ligand is a folic acid fragment or an analog thereof.

Clause SSS. The conjugate of any one of clauses LL-OO, wherein the targeting ligand is 5-methyltetrahydrofolate.

Clause TTT. The conjugate of clause MM, wherein at least one of Lₐ, L_{b}, L_{c}, and L_{d} each independently comprise: (—CH₂CH₂—O—)ₙ, where n is an integer between and including 1 and 32, an alkyl group, a peptide, a peptidoglycan, or a combination of two or more of the foregoing.

Clause UUU. The conjugate of clause NN, wherein at least one of Lₐ, L_{b}, and L_{c}, each independently comprise: (—CH₂CH₂—O—)ₙ, where n is an integer between and including 1 and 32, an alkyl group, a peptide, a peptidoglycan, or a combination of two or more of the foregoing.

Clause VVV. The conjugate of clause TTT or UUU, wherein n is an integer between and including 1 and 16.

Clause WWW. The conjugate of clause MM or OO, wherein at least one of Lₐ, L_{b}, and L_{c}, and L_{d} comprises a peptide fragment or a peptidoglycan fragment.

Clause XXX. The conjugate of clause MM or NN, wherein at least one of Lₐ, L_{b}, and L_{c} comprises a peptide fragment or a peptidoglycan fragment.

Clause ZZZ. The conjugate of clause MM or OO, wherein Lₐ, L_{b} and L_{c} L_{d} each independently comprise a C₂-C₁₈ alkyl group.

Clause AAAA. The conjugate of clause LL or NN, wherein Lₐ, L_{b} and L_{c} each independently comprise a C₂-C₁₈ alkyl group.

Clause BBBB. A conjugate of the formula:

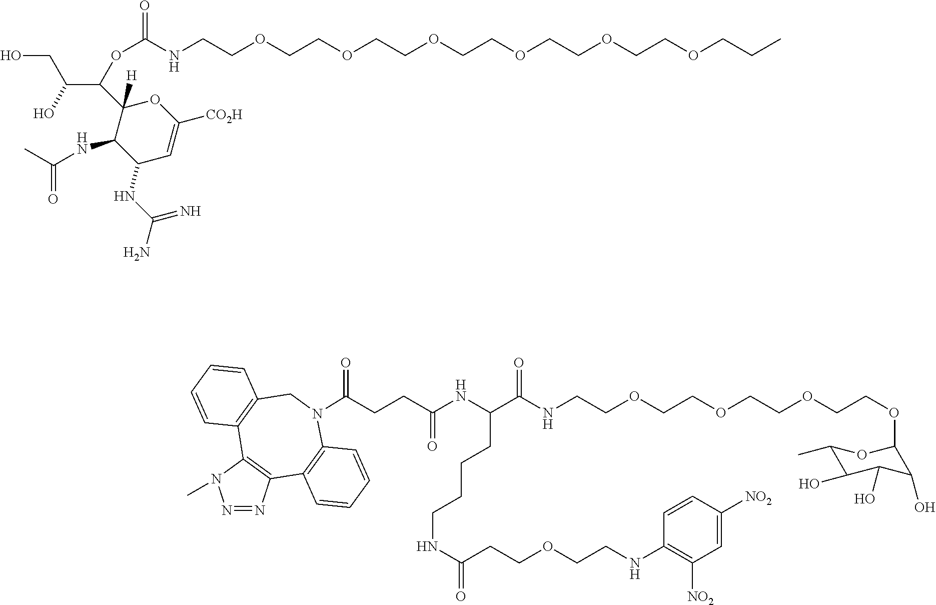

or a pharmaceutically acceptable salt thereof.

Clause CCCC. A conjugate of the formula:

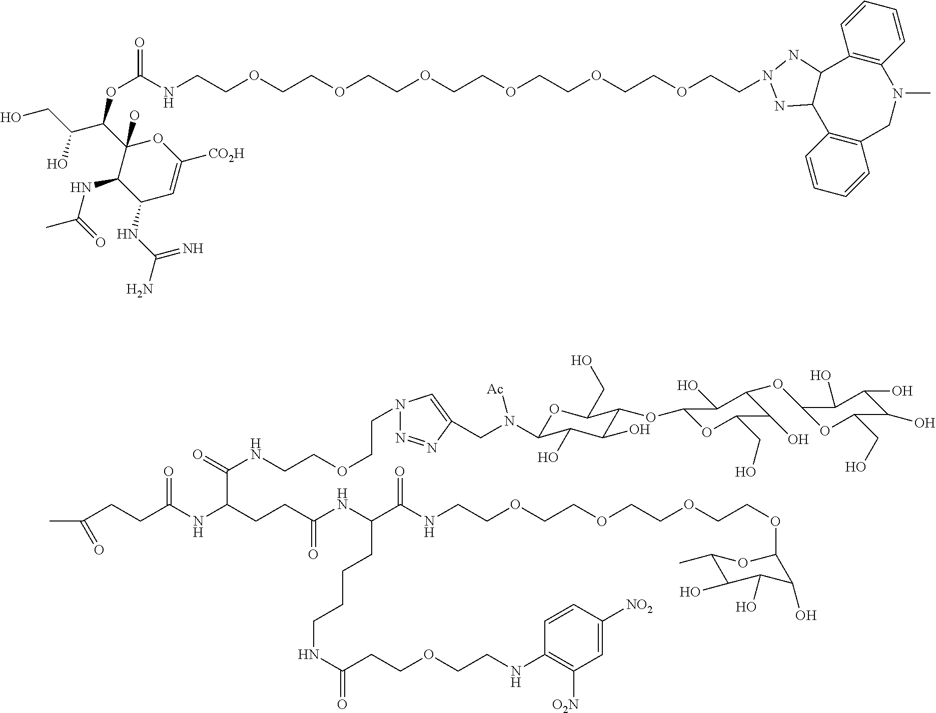

or a pharmaceutically acceptable salt thereof.

Clause DDDD. The conjugate of clause AAAA or BBBB, in which one or more of the —OH groups are independently replaced with a thiol, a phosphate, or a phosphante ester.

Clause FEEL. The conjugate of clause AAAA or BBBB, in which one or more of the —OH groups are replaced with —OC(═O)R, wherein R is an alkyl group.

Clause FFFF. The conjugate of clause AAAA or BBBB, in which one or more of the —OH groups are replaced with —OC(═O)R, wherein R is a C₁-C₆ alkyl group.

Clause GGGG. The conjugate of clause AAAA or BBBB, in which the amine (—NH₂) group is replaced with —OC(═O)R₂, and R₂ is an alkyl group.

Clause HHHH. The conjugate of clause AAAA or BBBB, in which the amine (—NH₂) group is replaced with —OC(═O)R₂, and R₂ is a C₁-C₆ alkyl group.

Clause IIII. The conjugate of clause AAAA or BBBB, in which the carboxyl (—COOH) group is replaced with —OC(═O)R₃, wherein R₃ is an alkyl group.

Clause JJJJ. The conjugate of clause AAAA or BBBB, in which the carboxyl (—COOH) group is replaced with —OC(═O)R₃, wherein R₃ is a C₁-C₆ alkyl group.

Clause KKKK. A conjugate of the formula:

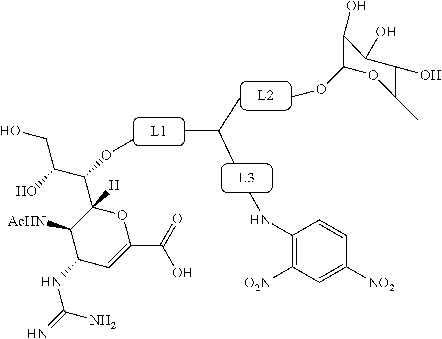 [SMILES: COC1OC(C)C(O)C(O)C1O ; CO[C@H]([C@@H]1OC(C(=O)O)=C[C@H](NC(=N)N)[C@H]1NC(C)=O)[C@H](O)CO ; CNc1ccc([N+](=O)[O-])cc1[N+](=O)[O-]]

or a pharmaceutically acceptable salt thereof, wherein L1, L2 and L3 are linkers.

Clause LLLL. The conjugate of clause JJJJ, wherein one or more of L1, L2 and L3 comprises (—CH₂CH₂—O—)ₙ wherein n is an integer between and including 1 and 16.

Clause MMMM. The conjugate of clause JJJJ or KKKK, wherein L1, L2 and L3 each independently comprise a C₂-C₁₈ alkyl group, a peptide fragment, or a peptidoglycan fragment.

Clause MMMM. A conjugate of the formula:

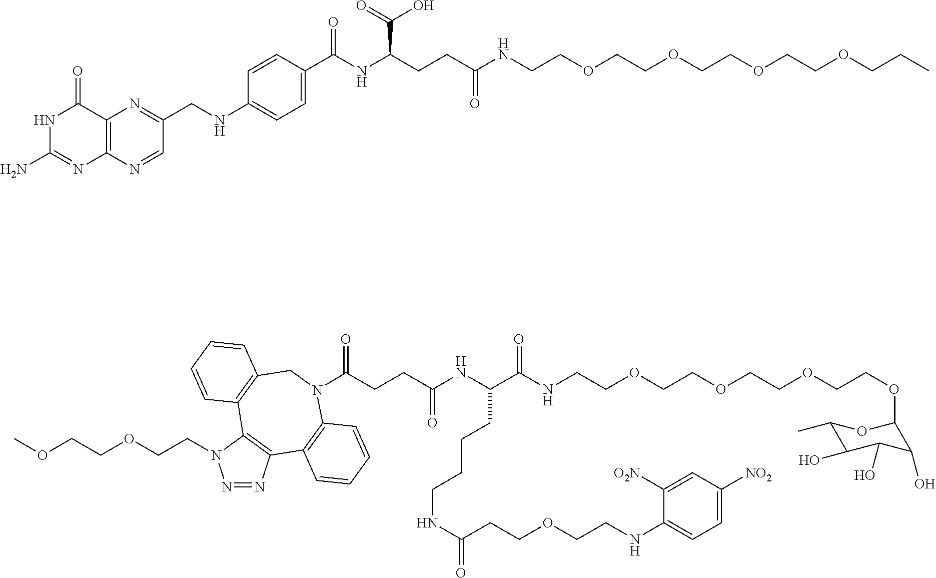

or a pharmaceutically acceptable salt thereof.

Clause NNNN. The conjugate of any one of clauses AAAA-MMMM, further complexed to one or more antibodies in vivo.

Clause OOOO. A pharmaceutical composition comprising a conjugate of any one of clauses A-MMMM and a pharmaceutically acceptable excipient.

Clause PPPP. A method of treating a viral infection in a subject comprising administering to the subject an effective amount of the conjugate of any one of clauses A-MMMM or a pharmaceutical composition of clause OOOO.

Clause QQQQ. The method of clause PPPP, further comprising administering to the subject autologous antibodies or allogeneic immunoglobulin G (IgG) antibodies.

Clause RRRR. The method of clause PPPP, wherein the viral infection is influenza.

Clause SSSS. The method of any one of clauses PPPP—RRRR, wherein the conjugate or the pharmaceutical composition is administered orally.

Clause TTTT. The method of clause PPPP, wherein the conjugate or the pharmaceutical composition is administered once daily.

Clause UUUU. The method of clause PPPP, wherein the conjugate or the pharmaceutical composition is administered more than once daily.

Clause VVVV. The method of clause PPPP, wherein the conjugate or the pharmaceutical composition is administered twice daily.

Clause WWWW. A method of treating cancer in a subject comprising administering to the subject an effective amount of the conjugate of any one of clauses A-MMMM or a pharmaceutical composition of clause OOOO.

Clause XXXX. The method of clause WWWW, further comprising administering to the subject autologous antibodies or allogeneic IgG antibodies.

Clause YYYY. The method of clause WWWW, wherein the cancer is a hot cancer.

Clause ZZZZ. The method of clause WWWW, wherein the cancer is renal cancer, lung cancer, or colorectal cancer.

Clause AAAAA. The method of any one of clauses PPPP—ZZZZ, wherein the conjugate or the pharmaceutical composition is administered orally or intravenously.

Clause BBBBB. The method of any one of clauses PPPP—ZZZZ, wherein the conjugate or the pharmaceutical composition is administered once daily.

Clause CCCCC. The method of any one of clauses PPPP—ZZZZ, further comprising administering a second therapy to the subject, wherein the second therapy comprises chemotherapy, sunitinib, a PD-1 inhibitor, or a PDL-1 inhibitor.

Clause DDDDD. A method for activating an immune response in a subject comprising administering to the subject an effective amount of the conjugate of any one of clauses A-MMMM or a pharmaceutical composition of clause OOOO.

Clause EEEEE. The method of clause DDDDD, wherein the immune response is an innate immune response.

Clause FFFFF. The method of clause DDDDD, wherein the immune response is activated in a targeted area of the subject, wherein the targeted area is a tumor microenvironment or a location of a virus replication site.

Clause GGGGG. The method of any one of clauses DDDDD-FFFFF, further comprising administering to the subject autologous antibodies or allogeneic IgG antibodies.

Clause HHHHH. The method of clause FFFFF, wherein administration of the effective amount of the conjugate or the pharmaceutical composition induces reprogramming of M2-type macrophages to M1-type macrophages in the targeted area.

Clause IIIII. The conjugate of clause A, wherein at least two of the Hs can each bind a different antibody when brought into contact with antibodies in vivo.

Clause JJJJJ. The conjugate of clause A, wherein two of the Us can each bind a different antibody when brought into contact with antibodies in vitro.

### List of Abbreviations

| | |
|---|---|
| MeOH | methanol |
| EtOH | ethanol |
| EtOAc | ethyl acetate |
| DCM | dichloromethane |
| ACN | acetonitrile |
| THF | tetrahydrofuran |
| TFA | trifluoroacetic acid |
| TIPS | triisopropylsilane |
| Py | pyridine |
| TEA | triethyl amine |
| DIPEA | N,N-diisopropylethylamine |
| HATU | 1-[bis(dimethylamino)-methylene]-1H-1,2,3- |
| | triazolo[4,5-b]pyridinium 3-oxid hexafluorophosphate |
| Ac | Acetyl |
| Ac2O | Acetic anhydride |
| DBCO-NHS | Dibenzocyclooctyne-N-hydroxysuccinimidyl ester |
| DBCO | Dibenzocyclooctyne |
| DCC | N,N′-Dicyclohexylcarbodiimide |
| DMSO | dimethylsulfoxide |
| BF3 Et2O | boron trifluoride etherate |
| HATU | 1-[bis(dimethylamino)-methylene]-1H-1,2,3- |
| | triazolo[4,5-b]pyridinium |
| PyBOB | benzotriazole-1-yl-oxy-tris-pyrrolidino-phosphonium |
| | hexafluorophosphate |
| NHS | N-hydroxysuccinimide |
| M | molar |
| DMAP | N,N-Dimethyl aminopyridine |
| PPh₃ | triphenyl phosphine |
| PEG | polyethylene glycol |
| ^{t}Bu | tert-butyl |
| NH₄OAc | ammonium acetate |
| Equiv. | equivalence |
| aq. | aqueous |
| min | minutes |
| g | grams |
| mg | milligram |
| mL | milliliter |
| mmol | millimoles |
| Qty. | quantity |
| LC-MS | liquid chromatography-mass spectrometry |
| ESI | electron spray ionization |
| m/z | mass/charge ratio |
| pTSOH | p-toluene sulfonic acid |
| Na₂CO₃ | sodium carbonate |
| NaOMe | sodium methoxide |
| Zan | zanamivir |
| Rha | rhamnose |
| DNP | dinitrophenyl |
| Fol | Folate |
| FR | Folate receptor |
| XO | Xofluza ® |
| TAMI | Tamiflu ® |
| IN | intranasal |
| IV | intravenous |
| OG | oral gavage |
| SC | subcutaneous |

### EXAMPLES

The following examples serve to illustrate the present disclosure. The examples are not intended to limit the scope of the claims in any way.

### Example 1

### Synthesis of Zan-PEG₆-DNP-Rhamnose (Zan-Dual Hapten) Drug Conjugate (Compound 24)

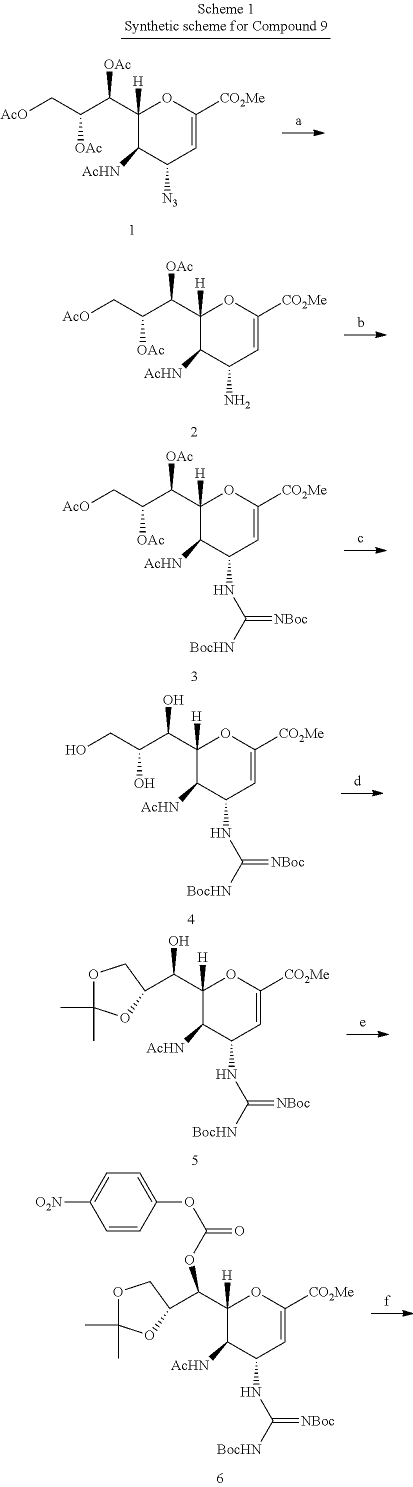

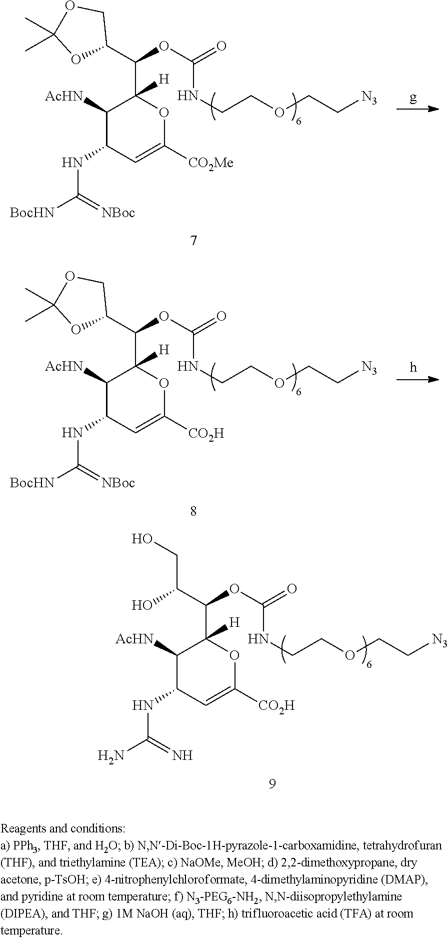

### Synthetic Scheme for Compound 12

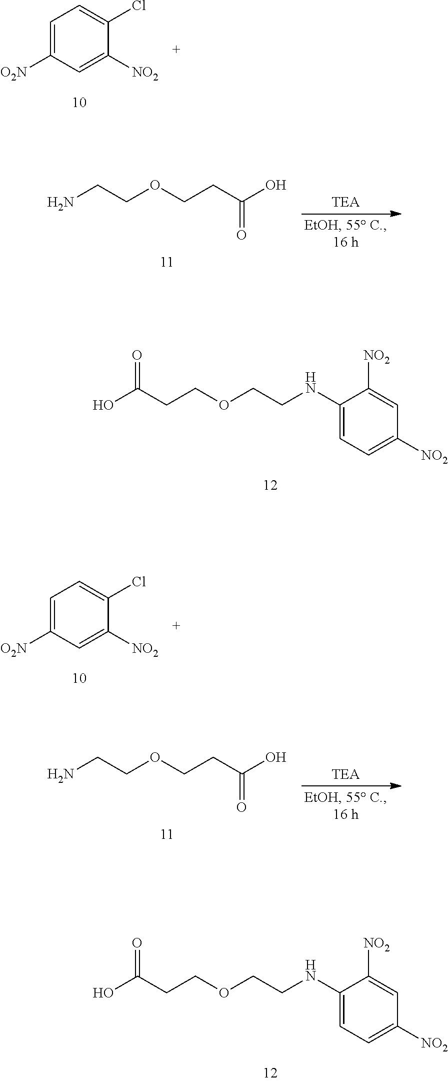 [SMILES: O=[N+]([O-])c1ccc(Cl)c([N+](=O)[O-])c1 ; NCCOCCC(=O)O ; O=C(O)CCOCCNc1ccc([N+](=O)[O-])cc1[N+](=O)[O-] ; O=[N+]([O-])c1ccc(Cl)c([N+](=O)[O-])c1 ; NCCOCCC(=O)O ; O=C(O)CCOCCNc1ccc([N+](=O)[O-])cc1[N+](=O)[O-]]

### Synthetic Scheme for Compound 16

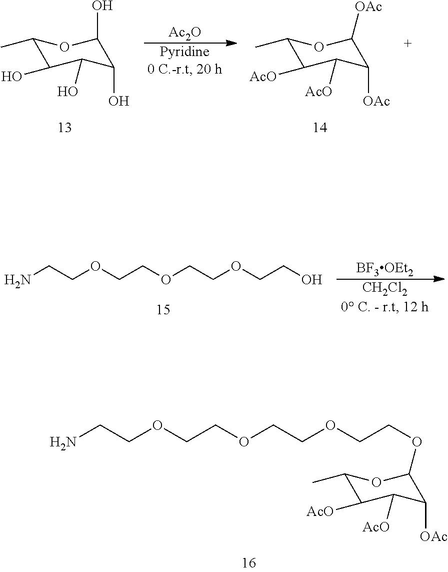

### Synthetic Scheme for Zan-DNP-Rhamnose (Compound 24)

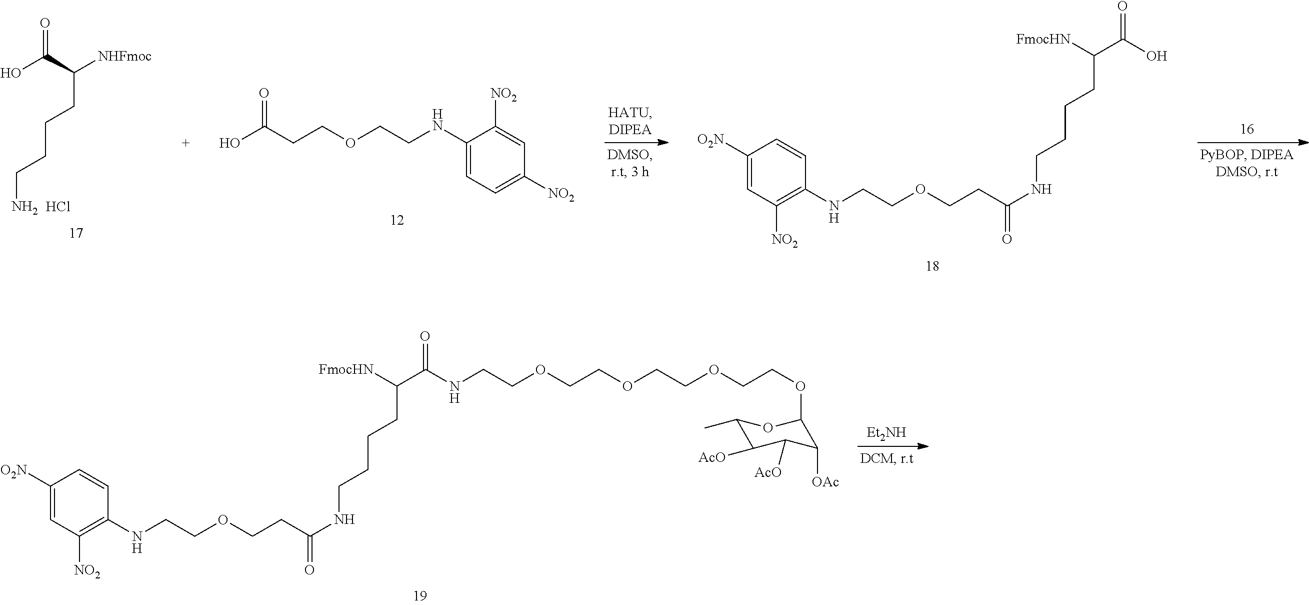

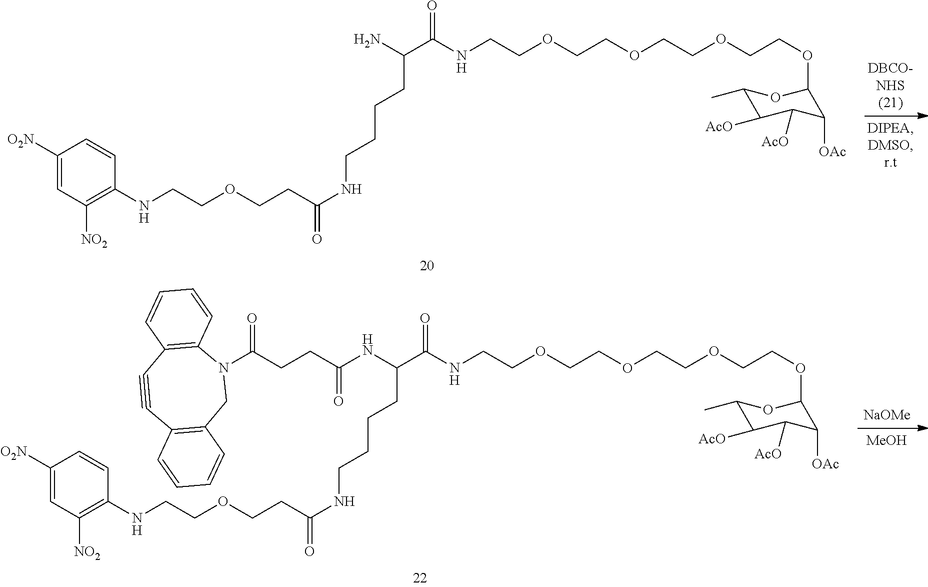

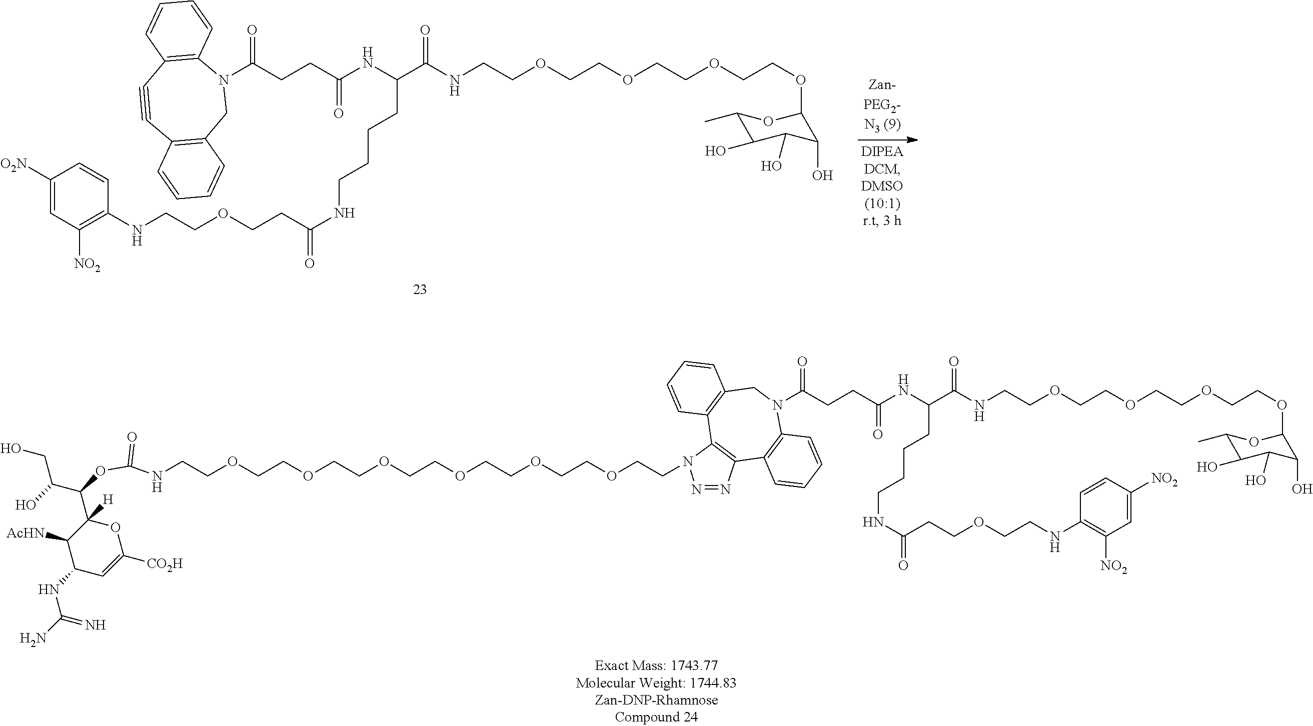

where Ac is acetate.

Synthesis of Compound 2: Zanamivir derivative 1 was prepared from sialic acid according to the literature methods reported in Chandler et al., Synthesis of the potent influenza neuraminidase inhibitor 4-guanidino Neu5Ac2en. X-Ray molecular structure of 5-acetamido-4-amino-2, 6-anhydro-3, 4, 5-trideoxy-Derythro-L-gluco-nononic acid, J Chem Society, Perkin Transactions 1: 1173-1180 (1995); Shidmoossavee et al., Chemical insight into the emergence of influenza virus strains that are resistant to Relenza, J Am Chem Soc 135: 13254-13257 (2013); and Ying & Gervay-Hague, One-Bead-One-Inhibitor-One-Substrate Screening of Neuraminidase Activity, ChemBioChem 6: 1857-1865 (2005). To a solution of zanamivir intermediate (1) (5 g, 11 mmol) in THF (40 mL), triphenylphosphine (3.67 g, 14 mmol, 1.27 equiv.) was added and the resulting solution was stirred at room temperature for 12 hours.

Subsequently, water (10 mL) was added and the solution was stirred at room temperature for another 24 hours. The reaction solution was then concentrated, and the crude product was purified by flash column chromatography on a Teledyne CombiFlash Rf+Lumen (silica gel column, 0-100% EtOAc in hexanes) to give 2 as a yellow powder. Product 2 was isolated, 2.89 g, yield, 61%.

Synthesis of Compound 3: Triethylamine (1.5 mL) was added to a solution of Compound 2 (2.74 g, 6.37 mmol) and N,N′-bis(tertbutoxycarbonyl)-1H-pyrazole-1-carboxamidine (2.57 g, 8.28 mmol, 1.30 equiv.) dissolved in THF (20 mL). The reaction mixture was stirred overnight at room temperature, and then concentrated and purified by flash column chromatography on a Teledyne CombiFlash Rf+Lumen (silica gel column, 0-100% EtOAc in hexanes) to give Compound 3 as a white solid (4.14 g, 97%).

Synthesis of Compound 5: NaOMe (2.9 mL, 0.5 M, 1.414 mmol) was added to a stirred solution of Compound 3 (4.225 g, 6.281 mmol) in anhydrous methanol (70 mL). The reaction mixture was then stirred for 1 hour. Dowex 50×W8 (H⁺) resin was added to neutralize the reaction mixture and filtered, concentrated to lead Compound 4 and was used for the next step without further purification.

To the Compound 4 in dry acetone (70 mL) was added 2,2-dimethoxypropane (7.7 mL, 6.54 g, 62.81 mmol, 10 equiv.), followed by p-toluenesulfonic acid (120 mg, 0.628 mmol, 0.1 equiv.) and the resulting mixture was stirred overnight at room temperature. The reaction mixture was then concentrated and purified by flash column chromatography on a Teledyne CombiFlash Rf+Lumen (silica gel column, 0-100% EtOAc in hexanes) to give Compound 5 as a white solid, Product isolated 2.4 g and yield, 65%.

Synthesis of Compound 6: To a solution of Compound 5 (1.46 g, 2.49 mmol) in pyridine (30 mL) was added 4-Dimethylaminopyridine (2.13 g, 17.43 mmol) and 4-nitrophenylchloroformate (3.51 g, 17.43 mmol). The reaction mixture was stirred ovemight at room temperature, and then concentrated and purified by flash column chromatography on a Teledyne CombiFlash Rf+Lumen (silica gel column, 0-100% EtOAc in hexanes) to give Compound 6 as a white solid (1.62 g, 87%).

Synthesis of Compound 7: To a solution of Activated-Zanamivir, Compound 6 (0.1 g, 0.13 mmol) in THF (2.7 mL) were added N₃-PEG₆-NH₂ (0.05 g, 0.14 mmol, 1.05 equiv.), followed by DIPEA (0.12 mL, 0.67 mmol, 5.0 equiv.) at room temperature under argon and stirred for overnight. Progress of the reaction was monitored by thin-layer chromatography (TLC) and LC/MS.

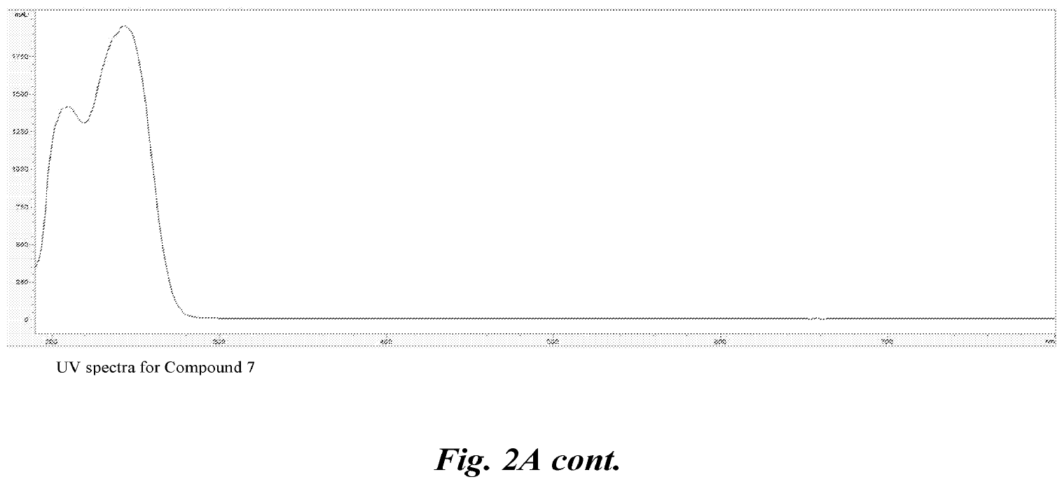

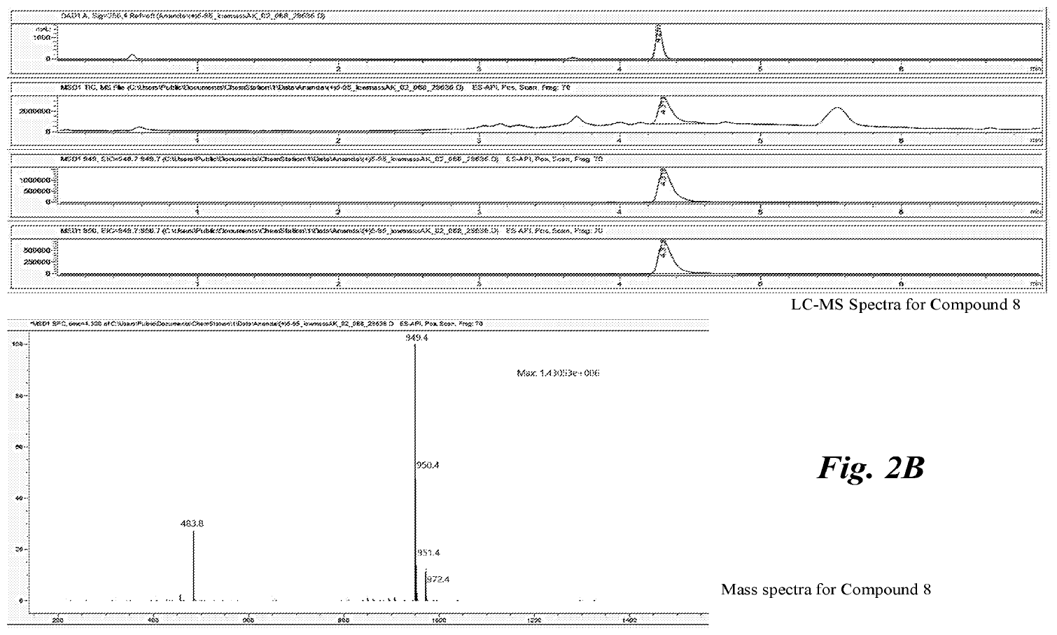

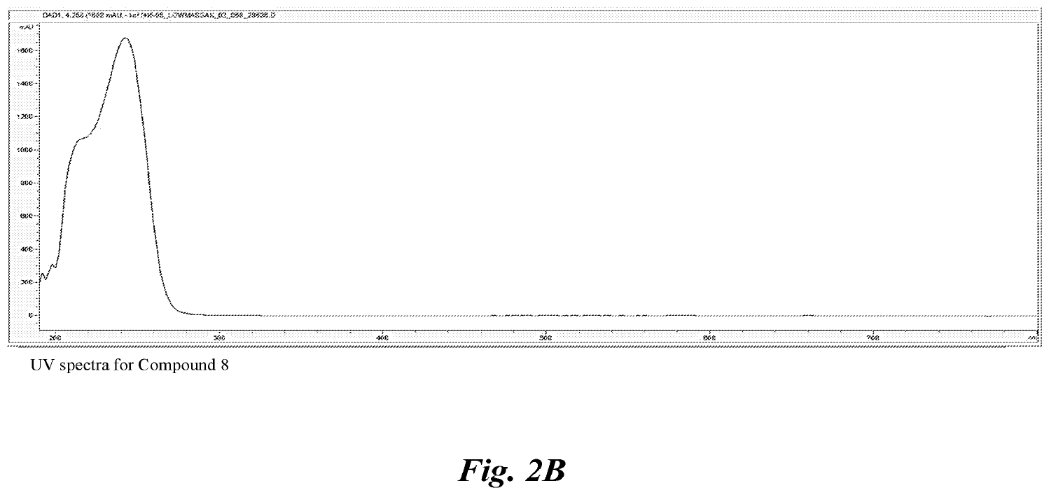

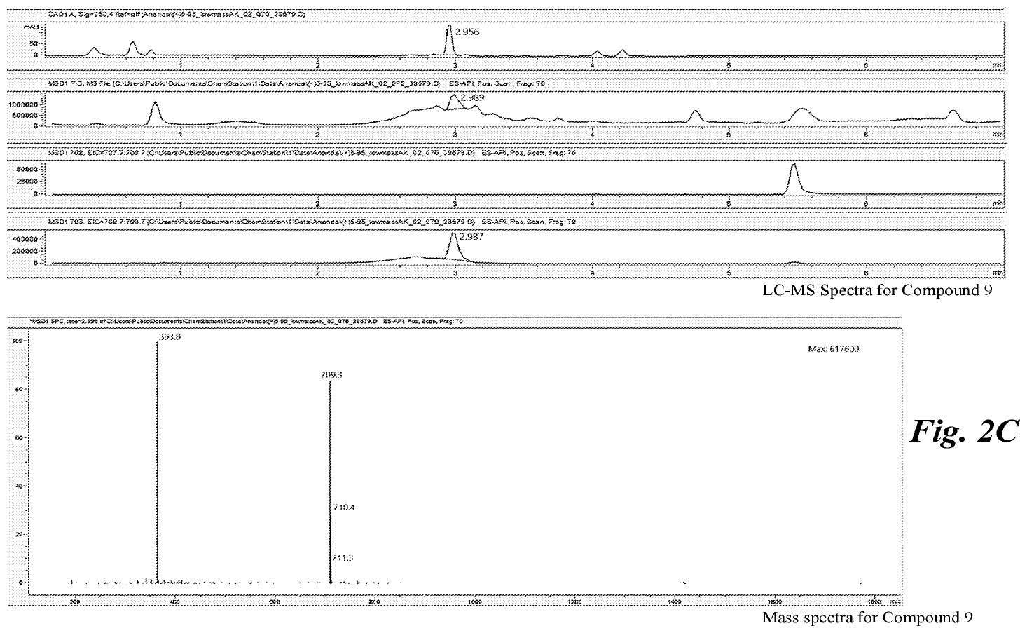

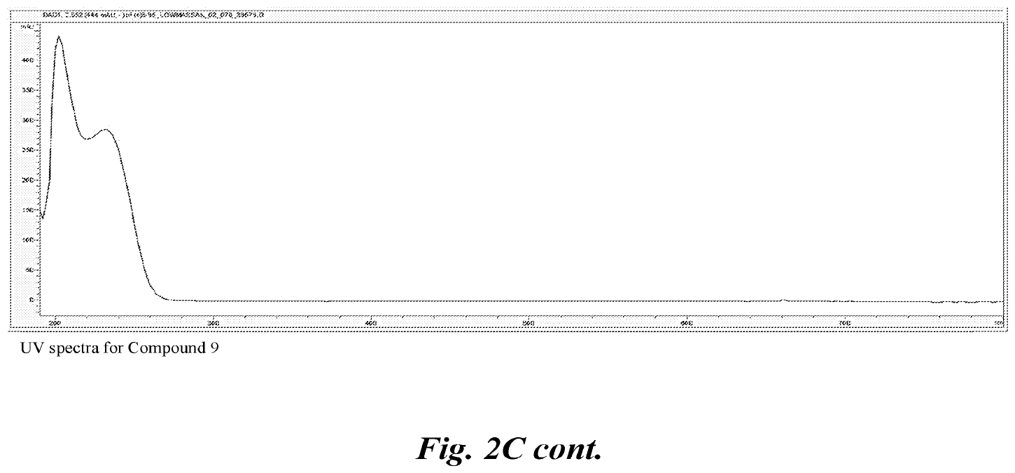

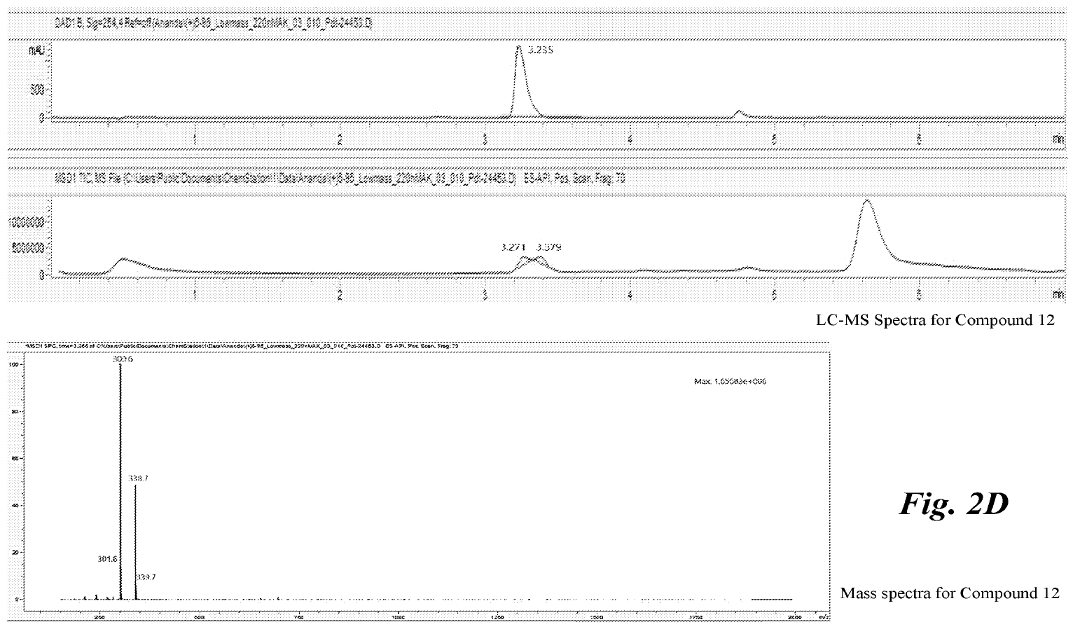

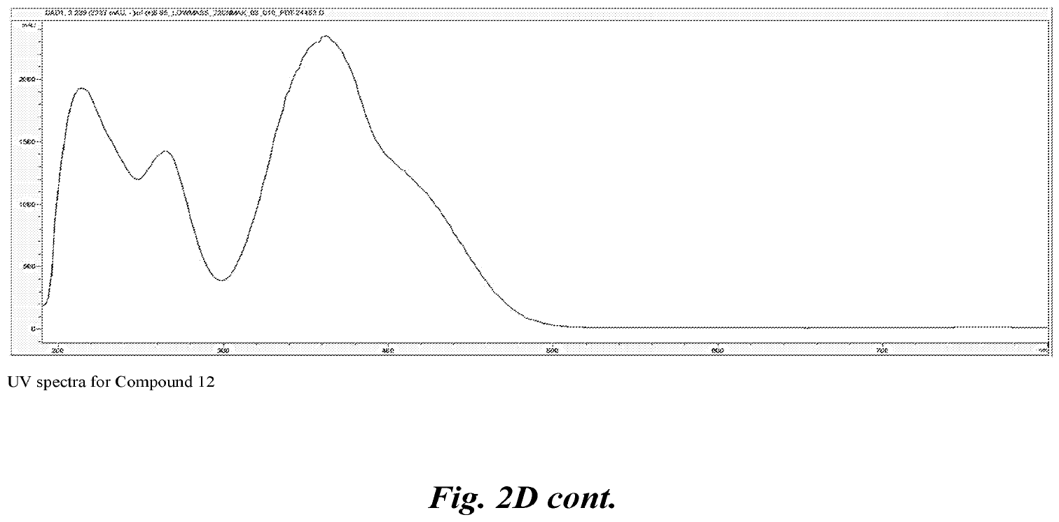

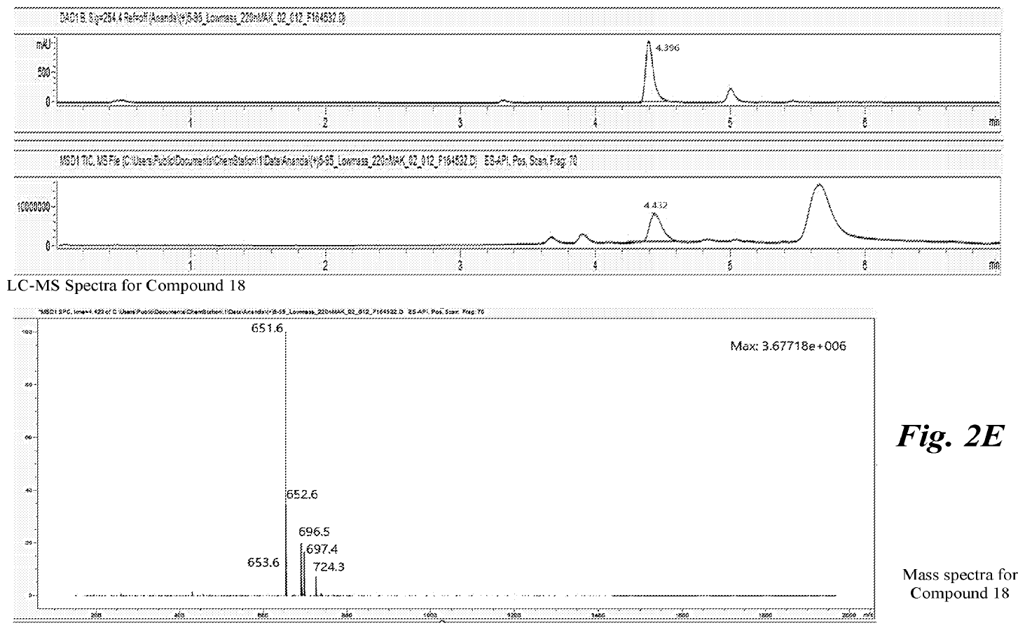

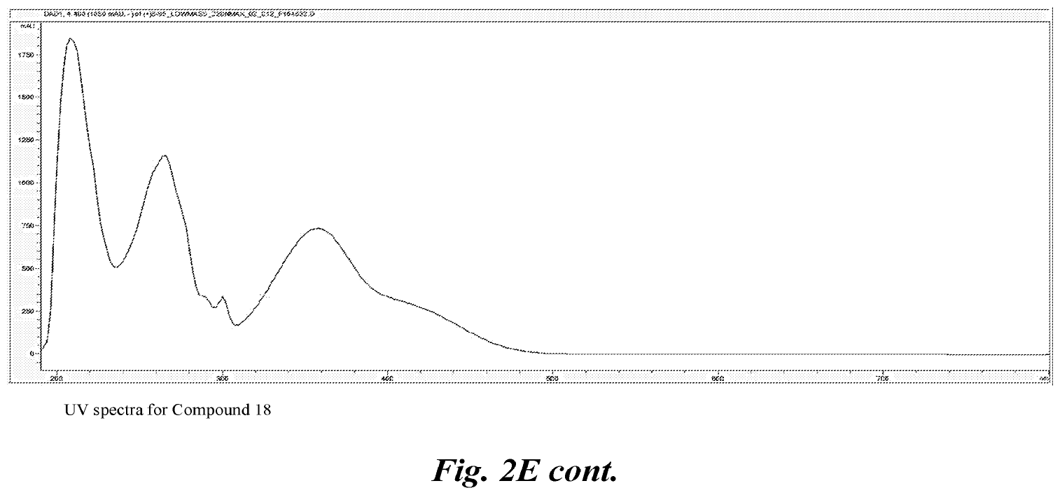

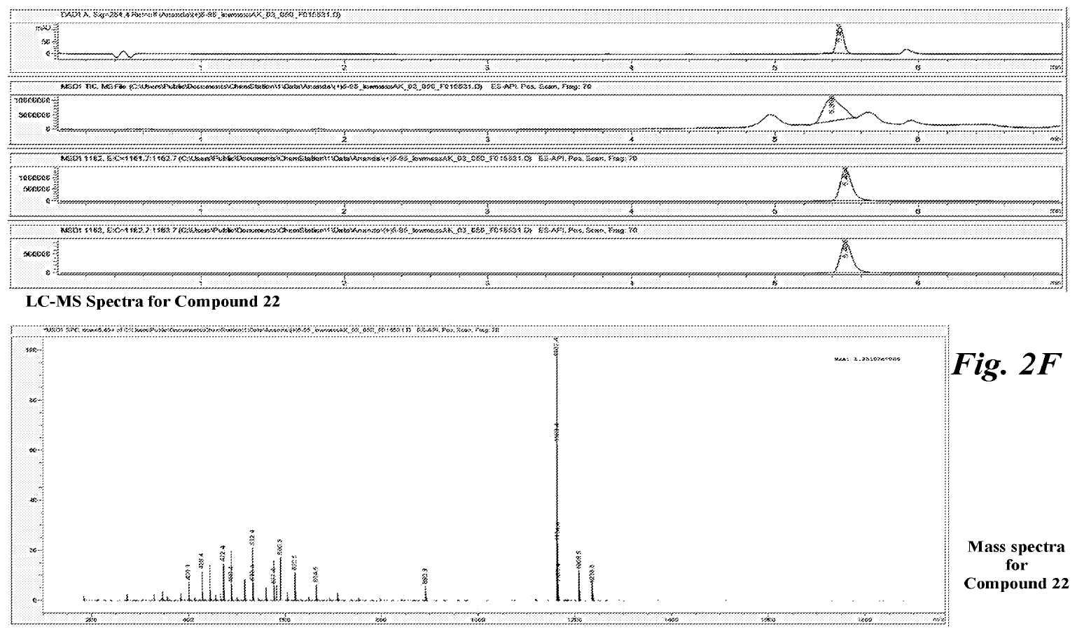

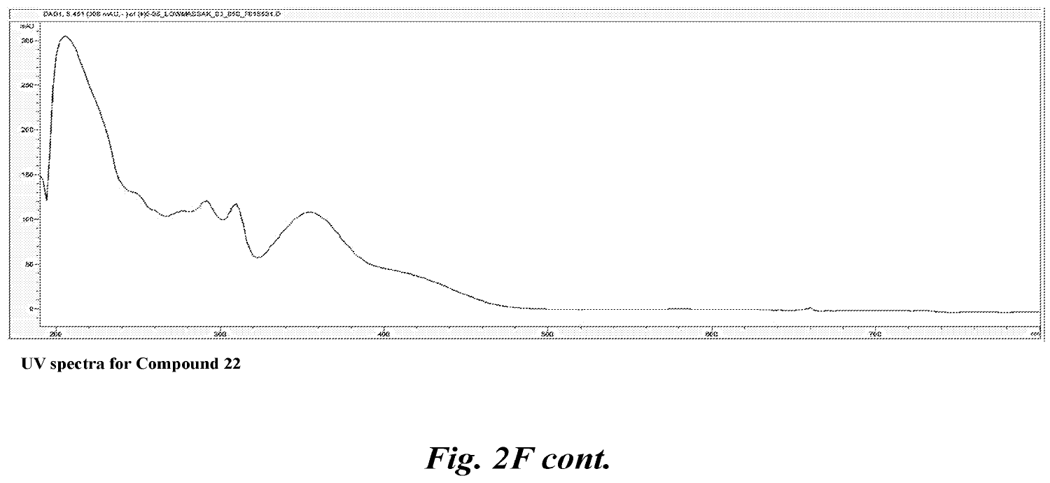

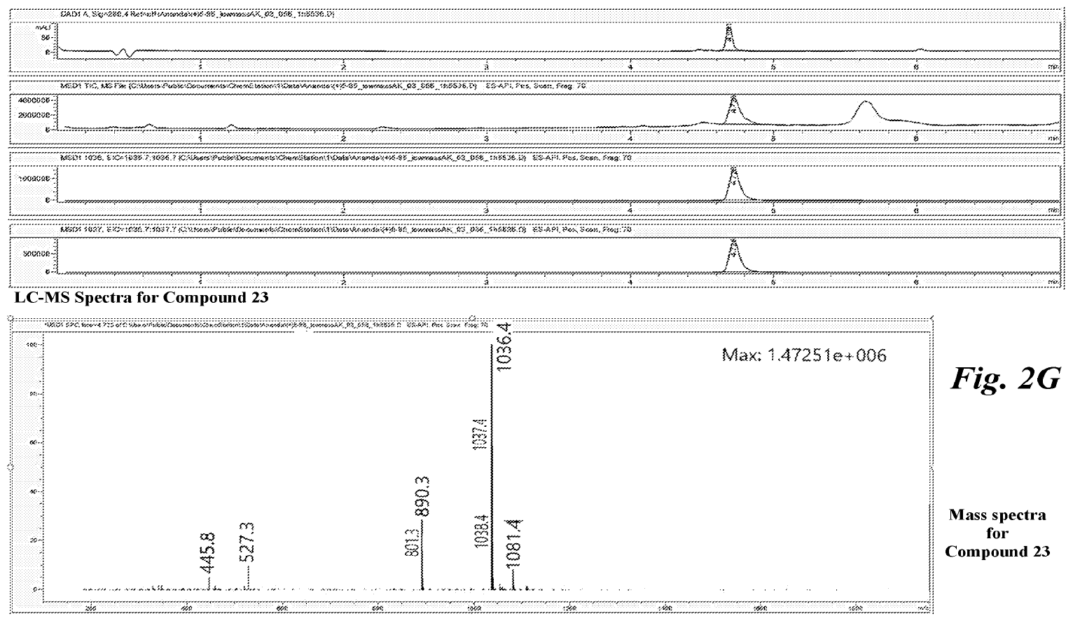

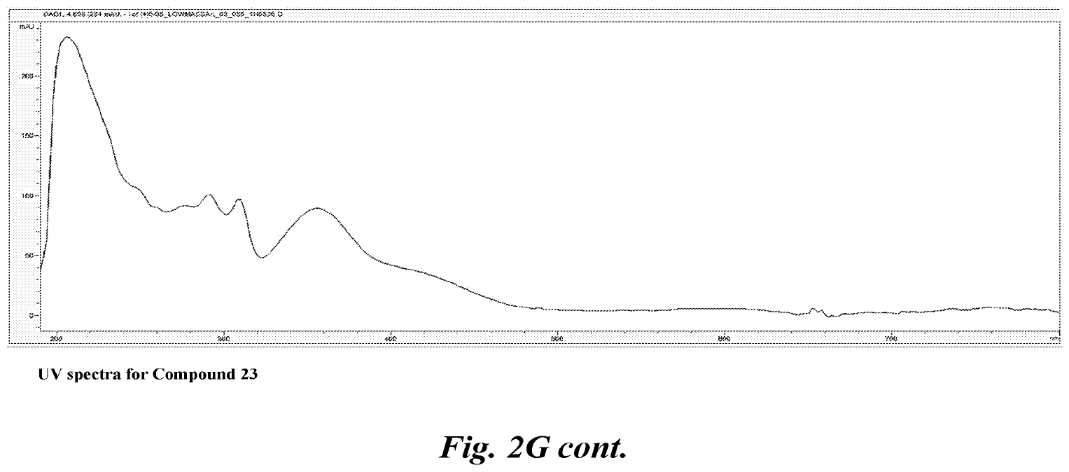

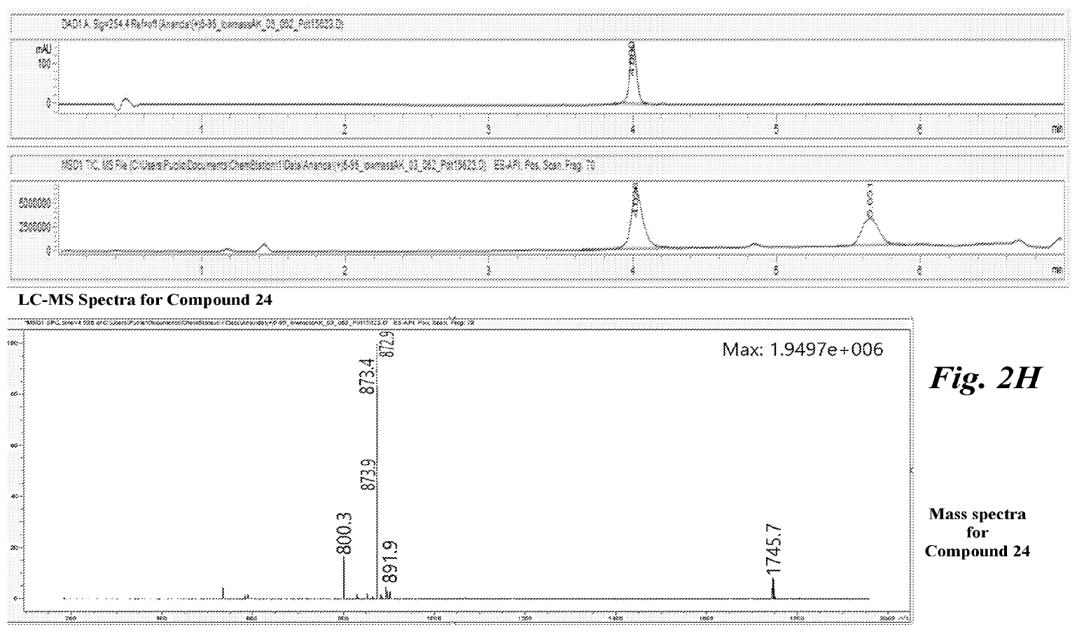

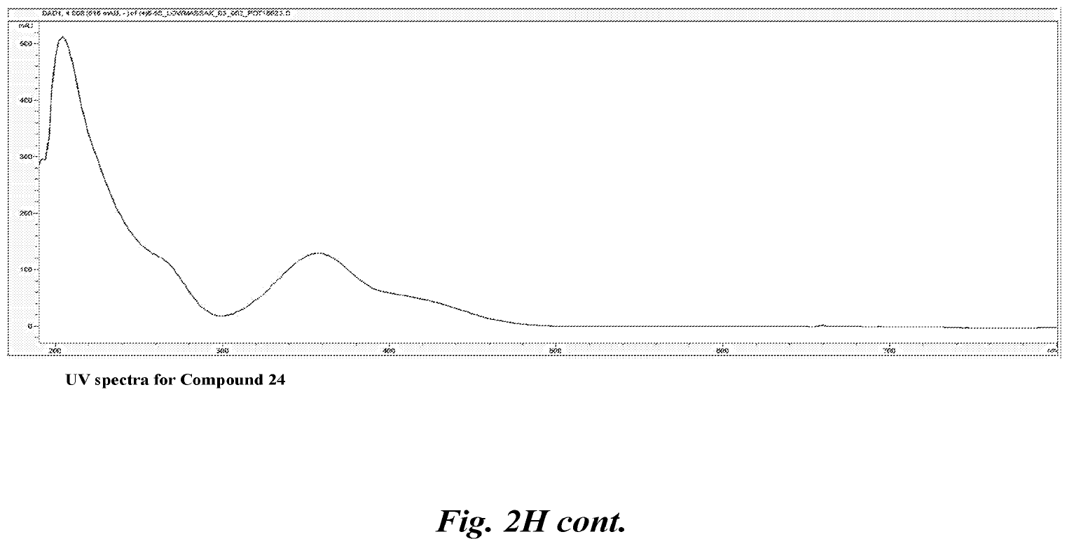

After completion of the reaction, the reaction mixture was concentrated under reduced pressure. The crude product was purified by flash column chromatography on a Teledyne CombiFlash Rf+Lumen (silica gel column, 0-10% MeOH in DCM) to give Compound 7 as a gummy solid (0.12 g, 93%) (See FIG. 2A).

Synthesis of Compound 9: Compound 7 (75 mg, 0.078 mmol) was dissolved in THF:MeOH (6:1, 1.5 mL) and treated dropwise with 1 M NaOH (100 L). The reaction mixture was stirred at room temperature for 1 hour at which time LC-MS analysis revealed that deprotection of methyl ester was completed. The reaction mixture was neutralized by adding Dowex® 50WX8 (H+) resin, filtered, and concentrated under reduced pressure. The intermediate crude product 8 was used directly for the next step without further purification. (See FIG. 2B).

To the intermediate crude compound 8, was added TFA (0.5 mL). The reaction mixture was stirred for an hour at room temperature and LC-MS was used to indicate the reaction was complete. TFA was removed by rotary evaporation under reduced pressure and the crude product was under vacuum to give Compound 9. The total yield over the 2 steps was 91%. (See FIG. 2C).

Synthesis of DNP-PEG₁-CO₂H (Compound 12): To a solution of 1-chloro-2,4-dinitrobenzene, Compound 10 (0.5 g, 2.47 mmol) and 3-(2-aminoethoxy)propanoic acid, Compound 11 (0.33 g, 2.47 mmol) dissolved in EtOH (25 mL) was added TEA (1.38 mL, 9.87 mmol). The reaction mixture was heated to 55° C. for 16 hours and reaction progress was monitored by LC-MS. After completion of the reaction, which was confirmed by disappearing of one of the starting materials (i.e., dinitrobenzene), the reaction mixture was cooled and concentrated under reduced pressure. The crude mixture was purified by flash column chromatography on Teledyne CombiFlash Rf+Lumen (silica-gel column, 0-20% methanol in DCM) to yield Compound 12 as a yellow solid, yield, 90%). LC-MS [M+H]+=299.98. (See FIG. 2D).

Synthesis of Compound 14: α-L-Rhamnose monohydrate (Compound 13, 1.0 g, 5.49 mmol) was dissolved in 9.2 mL of anhydrous pyridine. The solution was stirred in an ice bath and purged with nitrogen prior to the dropwise addition of acetic anhydrase (4.15 mL, 43.92 mmol, 8.0 equiv.). The reaction mixture was brought to room temperature slowly and monitored via TLC (Hexane/EtOAc, 65:35) and LC-MS, which indicated complete consumption of α-L-rhamnose monohydrate after 20 hours of reaction under an inert atmosphere. The reaction mixture was poured into ethyl acetate and extracted twice with 1.0 M HCl. The organic layer was washed with saturated sodium carbonate solution, water and brine and dried over anhydrous sodium sulfate, and concentrated under vacuum. The resulting crude oil product (Compound 14) (yield, 98%) was used for the next step.

Synthesis of Compound 16: 1,2,3,4-Tetra-O-acetyl-α-L-rhamnose (Compound 14, 0.50 g, 1.50 mmol) was dissolved in DCM (7.5 mL) prior to addition of amino-PEG₄-OH (Compound 15, 0.35 g, 1.81 mmol, 1.2 equiv.) under an inert atmosphere. The reaction flask was placed in an ice bath, and boron trifluoride diethyletherate (0.56 mL, 4.51 mmol, 3.0 equiv.) was added dropwise over 30 minutes. The reaction mixture was stirred at ice bath for 2 hours before the reaction was allowed to reach room temperature. Monitoring via TLC (Hexane/EtOAc, 30:70, Rf=0.30) and LC-MS indicated complete consumption of 1,2,3,4-tetra-O-acetyl-α-L-rhamnose after 16 hours of reaction. The reaction mixture was poured into ice water and extracted with DCM.

The combined organic layers were washed twice with saturated sodium bicarbonate solution, water and brine and dried over anhydrous sodium sulfate and concentrated under vacuum. The resulting crude purified by silica gel column chromatography (Hexane/EtOAc, 20:80) and product (Compound 16) isolated, yield, 90%.

Synthesis of Compound 18: 1-[bis(dimethylamino)-methylene]-1H-1,2,3-triazolo[4,5-b]pyridinium 3-oxid hexafluorophosphate, HATU (0.11 g, 0.28 mmol, 0.85 equiv.) was added to a solution of 3-(2-((2,4-dinitrophenyl)amino)ethoxy)propanoic acid (Compound 12, 0.1 g, 0.33 mmol) in dimethyl sulfoxide (2 mL), followed by DIPEA (0.29 mL, 1.67 mmol, 5.0 equiv.) under argon atmosphere and stirred for 10 minutes at room temperature. Fmoc-Lys-OH·HCl (Compound 17, 0.11 g, 0.27 mmol, 0.8 equiv.) was added to the reaction mixture and stirred for 2-3 hours at room temperature. Progress of the reaction was monitored by LC-MS. After completion of the reaction was confirmed by LC-MS, the reaction mixture was quenched by adding water and extracted with ethyl acetate. The combined organic layer was washed with brine, dried over anhydrous sodium sulfate and concentrated under vacuum. The resulting crude was purified by silica gel column chromatography (Hexane/EtOAc, 20:80) and product (Compound 18) was isolated, yield, 70%. (See FIG. 2E).

Synthesis of Compound 19: To a solution of acid (Compound 18, 0.06 g, 0.09 mmol) and Rham(OAc)₃-PEG₄-NH₂ (Compound 16, 0.04 g, 0.09 mmol, 1.0 equiv.) in dimethyl sulfoxide (1.5 mL) was added benzotriazole-1-yl-oxy-tris-pyrrolidino-phosphonium hexafluorophosphate, PyBOP (0.05 g, 0.10 mmol, 1.1 equiv.), followed by DIPEA (0.081 mL, 0.46 mmol, 5.0 equiv.) under argon atmosphere at room temperature. Progress of the reaction was monitored by LC/MS.

After completion of the reaction was confirmed by LC-MS, the reaction mixture was quenched by adding water and extracted with ethyl acetate. The combined organic layer was washed with brine, dried over anhydrous sodium sulfate and concentrated under vacuum. The resulting crude was purified by silica gel column chromatography (0-10% MeOH in DCM) and the product (Compound 19) was isolated, yield, 93%.

Deprotection of -Fmoc group (Compound 20): To Compound 19 (0.01 g, 0.01 mmol) in dry DCM (0.2 ml) was added DEA (100 uL) under argon at room temperature. The solution was stirred for 1 hour at room temperature until the reaction was completed as demonstrated by LC-MS. The DEA was removed by rotary evaporation under reduced pressure and the crude product was precipitated in diethyl ether to give Compound 20 as a yellow solid with a quantitative yield. LC-MS [M+H]+=876.1.

Synthesis of Compound 22: To a solution of DBCO-NHS (Compound 21, 0.012 g, 0.029 mmol, 1.05 equiv.) and compound 20 (0.024 g, 0.027 mmol, 1.0 equiv.) dissolved in DCM:DMSO (10:1, 0.6 mL) was added N,N-DIPEA (0.048 μl, 0.27 mmol, 10 equiv.) dropwise under argon.

The reaction mixture was stirred at room temperature for 2-3 hours and progress of the reaction was monitored by LC-MS. After completion of the reaction, the solvent was evaporated using rota-evaporator under reduced pressure and the crude was purified by silica-gel column chromatography on Teledyne CombiFlash Rf+Lumen (0-10% methanol in DCM) to afford Compound 22 as a yellowish solid, yield, 93%). LC-MS [M+H]+=1163.2. (See FIG. 2F).

Hydrolysis of OAc groups (Compound 23): Compound 22 (0.025 g, 0.022 mmol) was dissolved in anhydrous MeOH (1 mL) and treated dropwise with 0.5 M NaOH (25 μL). The reaction mixture was stirred at room temperature for 1 hour at which time LC-MS analysis revealed that the product formation. After completion of the reaction, the reaction mixture was neutralized by adding Dowex® 50WX8 (H+) resin, filtered, and concentrated under reduced pressure. The yellowish crude product (Compound 23) was used directly for the next step without further purification. LCMS [M+H]+=1037.3. (See FIG. 2G).

Synthesis of zan-PEG₆-DNP-Rhamnose (Compound 24): To a solution of zanamivir-PEG₆-azide (Compound 9) (2.4 mg, 0.003 mmol) and Compound 23 (3.5 mg, 0.003 mmol, 1.0 equiv.) in dry DCM:DMSO (10:1, 0.2 mL) was added DIPEA (6 μl, 0.03 mmol, 10 equiv.) dropwise under argon. The reaction mixture was stirred at room temperature (room temperature) for 2 hours and the progress of the reaction was monitored by LC-MS. After completion of the reaction, solvent was removed by rotary evaporation under reduced pressure and the crude product was purified by prep-high-performance liquid chromatography (HPLC) on a C18 column (5-95% acetonitrile in aqueous 20 mM NH₄OAc, pH 7 buffer, 60 minutes, flow 7 mL/minute) to yield Zan-PEG₆-DNP-Rhamnose (Compound 24) as a yellow powder, yield, 50%). LC-MS [M+H]+=1745.9. (See FIG. 2H).

### Example 2

### Comparison of Compound 24 with Zan-Fc-WT and Zan-Fc-DLE

6-8 weeks old female BALB/c mice (n=5/group) were infected with 100 LD₅₀ of Influenza virus A/Puerto Rico/8/1934 (H1N1) (Catalog No. NR-348, BEI Resources, NIAID, NIH) on day 0 of the experiment. Mice (except the phosphate-buffered saline (PBS) only group) were given an intraperitoneal injection of 8 g/kg human intravenous immunoglobulin (IVIg) at 24 hours post-infection (hpi) to achieve humanized titer of anti-DNP and anti-Rhamnose antibodies at the time of test article administration. Mice were treated with test articles at 48 hpi. Zan-DNP-Rhamnose was given as a single intranasal dose of 1.5 μmol/kg. zan-Fc-WT and zan-Fc-DLE were given as a single intravenous dose of 10 μg/mouse (synthesis details below). Mice in the two control groups received PBS as placebo.

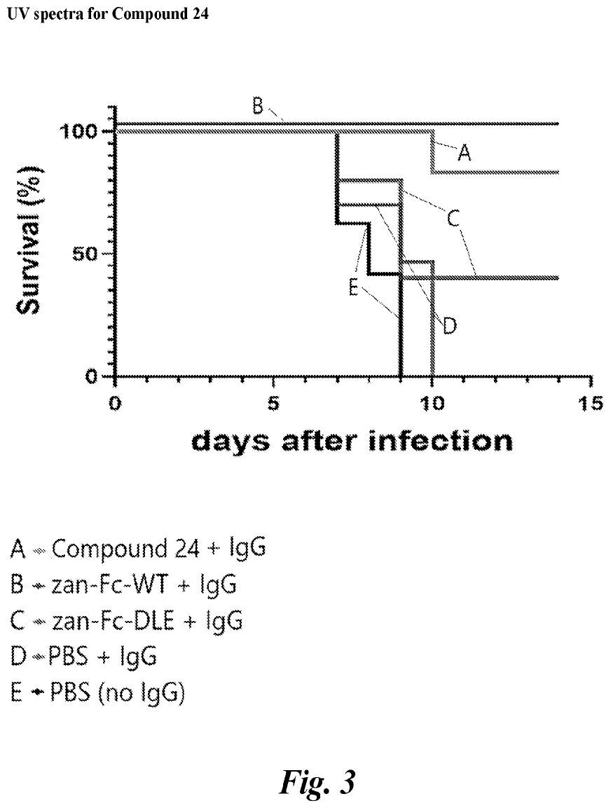

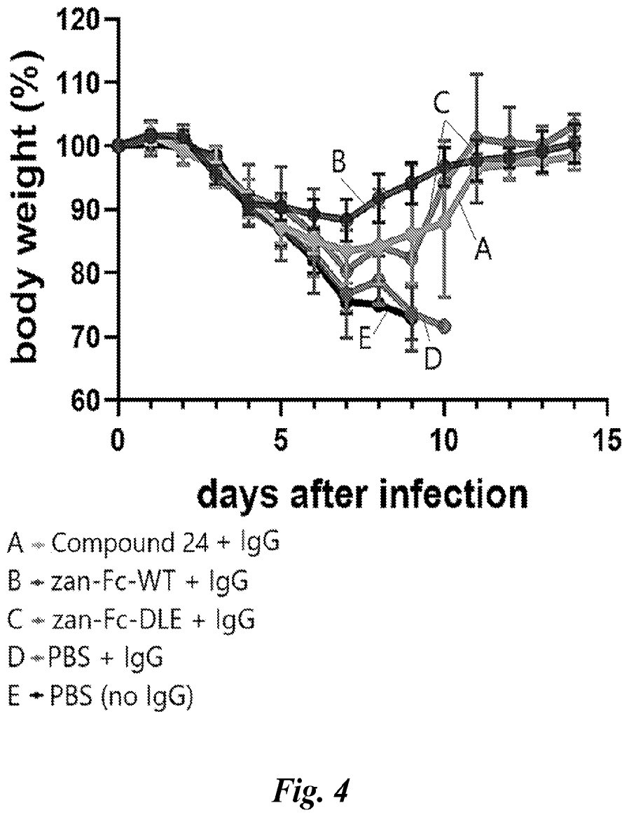

For evaluation of drug efficacy, mice were weighed and monitored daily for 14 days post-infection and counted as dead when they lost 25% of their body weight or were diagnosed as moribund. Results of this experiment are depicted in FIG. 3 and FIG. 4.

FIG. 3 is a graph of days after infection vs. survival (%) for mice (n=5/group) infected with 100 LD₅₀ of influenza A H1N1/PR8, removal of anti-influenza antibodies, intraperitoneal administration of IVIg (GAMUNEX®—C) at 24 hpi, and administration of conjugate at 48 hpi.

FIG. 4 is a graph of days after infection vs. body weight (%) for the mice treated as described in FIG. 3.

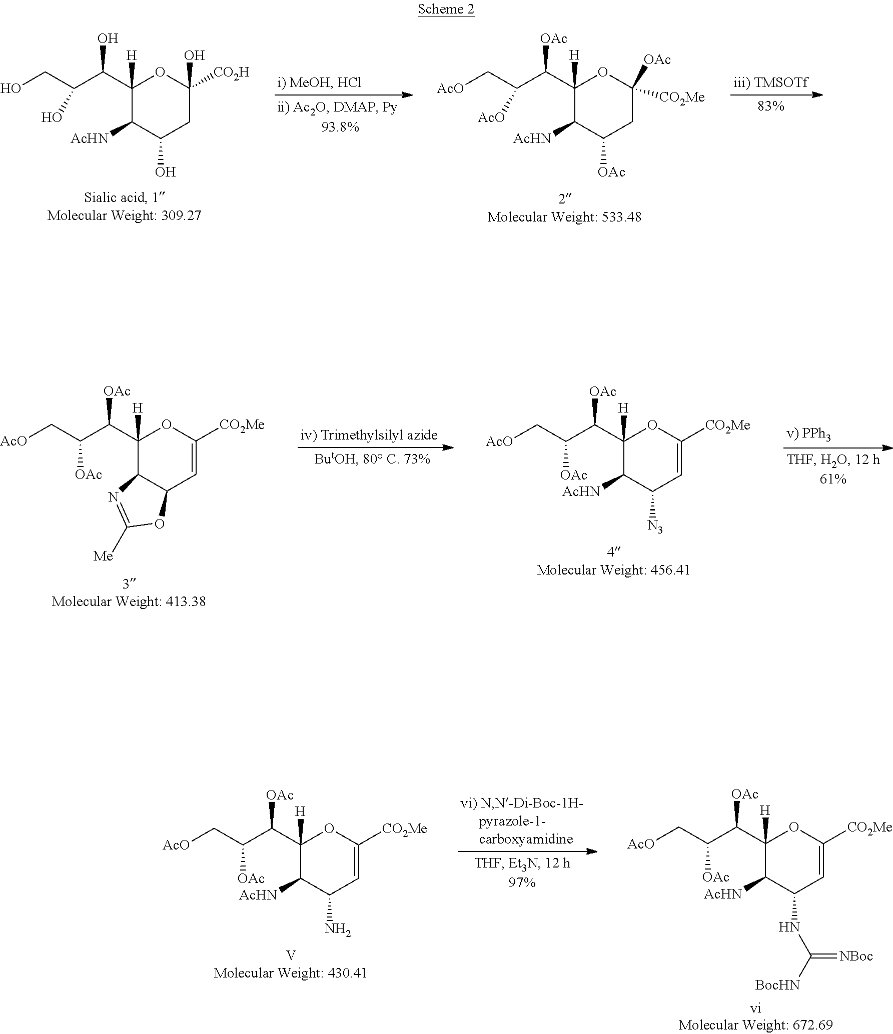

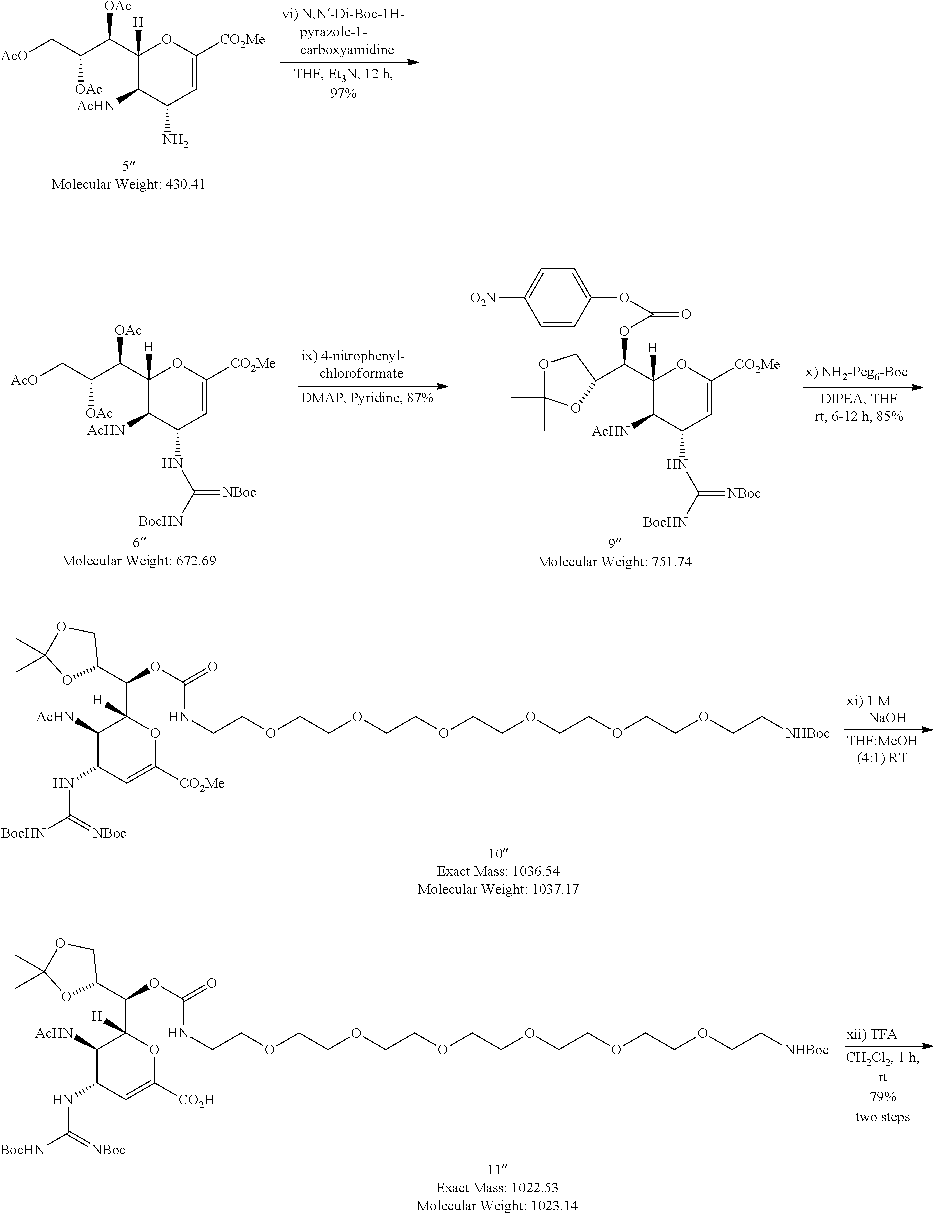

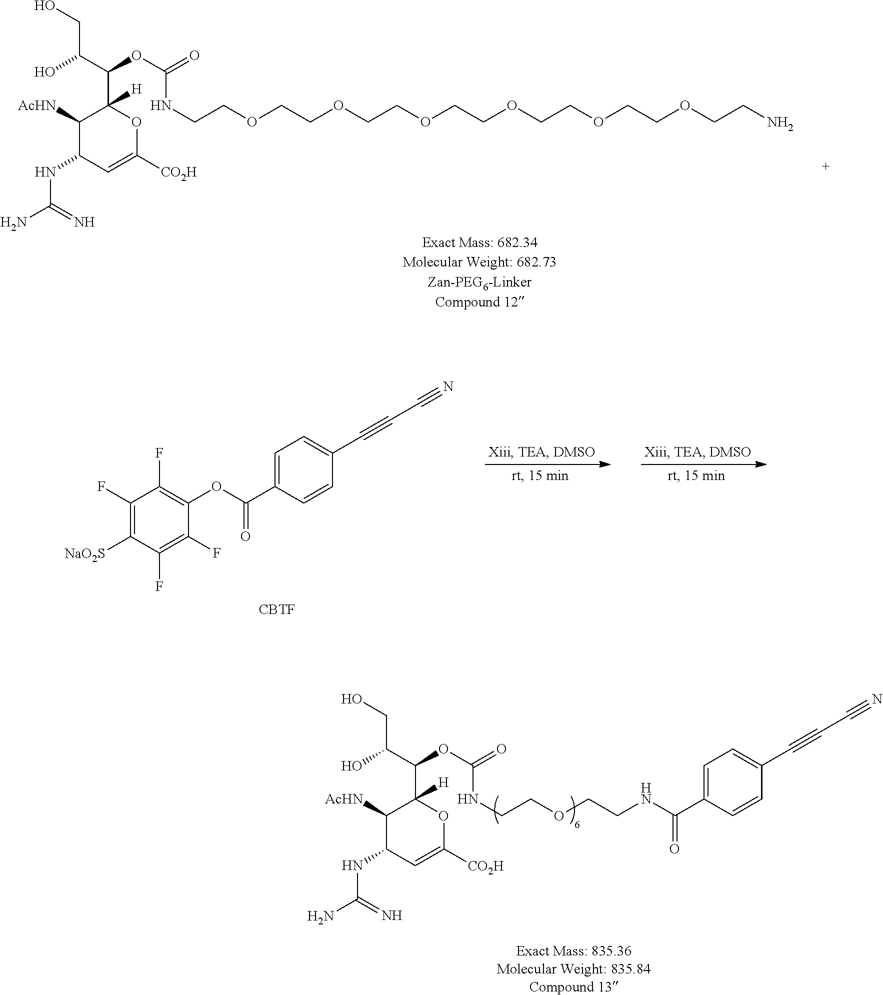

Zanamivir-azide, Compound 4″, was prepared from sialic acid according to previously reported literature methods (Chandler et al., Synthesis of the potent influenza neuraminidase inhibitor 4-guanidino Neu5Ac2en. X-ray molecular structure of 5-acetamido-4-amino-2,6-anhydro-3,4,5-trieoxy-D-erythro-L-gluco-nononic acid, J Chemical Society, Perkin Transactions 1: 1173-1180 (1995); Shidmoossavee et al., Chemical insight into the emergence of influenza virus strains that are resistant to Relenza, J American Chemistry Society 135(36): 13254-13257 (2013); Ying, L. & Gervay-Hague, One-bead-one-inhibitor-one-substrate screening of neuraminidase activity, ChemBioChem 6(10): 1857-1865 (2005).

Synthesis of Compound 5″: To a solution of zanamivir-azide (Compound 4″) (5 g, 11 mmol) in THF (40 mL), triphenylphosphine (3.67 g, 14 mmol, 1.27 equiv.) was added, and the resulting solution was stirred at rt for 12 hours. Subsequently, water (10 mL) was added, and the solution was stirred at rt for another 24 hours. The reaction solution was then concentrated, and the crude product was purified by flash column chromatography on a Teledyne CombiFlash Rf+Lumen (silica gel column, 0-100% EtOAc in hexanes) to give Compound 5″ as a yellow powder. Product (Compound 5″) isolated, 2.89 g; yield, 61.1%.

Synthesis of Compound 6″: To a solution of Compound 5″ (2.74 g. 6.37 mmol) and N,N′-bis(tertbutoxycarbonyl)-1H-pyrazole-1-carboxamidine (2.57 g, 8.28 mmol, 1.30 equiv.) dissolved in THF (20 mL) was added triethylamine (1.5 mL). The reaction mixture was stirred overnight at rt and then concentrated and purified by flash column chromatography on a Teledyne CombiFlash Rf+Lumen (silica gel column, 0-100% EtOAc in hexanes) to give Compound 7″ as a white solid (4.14 g, 97%).

Synthesis of Compound 8″: NaOMe (2.9 mL, 0.5 M, 1.414 mmol) was added to a stirred solution of compound (Compound 6) (4.225 g, 6.281 mmol) in anhydrous methanol (70 mL). The reaction mixture was then stirred for 1 hour. Dowex 50×W8 (H⁺) resin was added to neutralize the reaction mixture, and the mixture was filtered and concentrated to give lead Compound 7″, which was used for the next step without further purification.

To the Compound 7″ in dry acetone (70 mL) was added 2,2-dimethoxypropane (7.7 mL, 6.54 g, 62.81 mmol, 10 equiv.), followed by p-toluenesulfonic acid (120 mg, 0.628 mmol, 0.1 equiv.). The resulting mixture was stirred overnight at rt. The reaction mixture was then concentrated and purified by flash column chromatography on a Teledyne CombiFlash Rf+Lumen (silica gel column, 0-100% EtOAc in hexanes) to give Compound 8″ as a white solid (2.4 g, 65.2%).

Synthesis of Compound 9″: To a solution of Compound 8″ (1.46 g, 2.49 mmol) in pyridine (30 mL) were added 4-dimethylaminopyridine (2.13 g, 17.43 mmol) and 4-nitrophenylchloroformate (3.51 g, 17.43 mmol). The reaction mixture was stirred overnight at rt and then concentrated and purified by flash column chromatography on a Teledyne CombiFlash Rf+Lumen (silica gel column, 0-100% EtOAc in hexanes) to give activated-Zanamivir (Compound 9″) as a white solid (1.62 g, 87%).

Synthesis of Compound 10″: To a solution of activated-Zanamivir (Compound 9″) (0.2 g, 0.27 mmol) in THF (12.0 mL) were added tert-Boc-N-amido-PEG₆-amine (0.124 g, 0.29 mmol, 1.1 equiv.) followed by DIPEA (0.23 mL, 1.33 mmol, 5.0 equiv.) at rt under argon with stirring for 6-12 hour. Progress of the reaction was monitored by TLC and LC/MS. After completion of the reaction, the reaction mixture was concentrated under reduced pressure. The crude product was purified by flash column chromatography on a Teledyne CombiFlash Rf+Lumen (silica gel column, 0-20% MeOH in DCM) to give Compound 10″ as a gummy solid (234 mg, 85%).

Synthesis of Compound 12″: Compound 10″ (0.22 g, 0.21 mmol) was dissolved in THF (1.5 mL) and treated drop-wise with 1 M NaOH (0.6 mL). The reaction mixture was stirred at rt for 1 hour at which time LC-MS analysis revealed that deprotection of methyl ester was completed. The reaction mixture was neutralized by adding Dowex® 50WX8 (H+) resin, filtered, and concentrated under reduced pressure. The intermediate crude Compound 11″ was used directly for the next step without further purification.

TFA (1.5 mL) was added to the intermediate crude Compound 11″. The solution was stirred for 1 hour at rt until the reaction was completed as demonstrated by LC-MS. TFA was removed by rotary evaporation under reduced pressure and treated with diethyl ether (3×2 mL) and dried under vacuum to give Compound 12″ as a gummy product. The total yield over the 2 steps was 79%.

Synthesis of Compound 13″: To a solution of Compound 12″ (4 mg, 5.86 μmol) in DMSO (200 μL) were added sodium 4-((4-(cyanoethynyl)benzoyl)oxy)-2,3,5,6-tetrafluorobenzenesulfonate and CBTF (2.60 mg, 6.15 μmol, 1.05 equiv.), followed by triethylamine (8.20 L, 58.59 μmol, 10.0 equiv.), at rt under argon and stirring for 10-15 minutes. Progress of the reaction was monitored by TLC and LC-MS. After completion of the reaction, Compound 13″ was used directly for the next step without any purification (it is better to purify using silica gel column for conjugation with Fc protein).

For the LC-MS evaluations, the conditions were as follows: Column: XBridge BEH C18 Column, 130 Å, 3.5 μm, 3 mm×100 mm; Mobile phase: A: 20 mM ammonium bicarbonate buffer, pH 7; B; Acetonitrile (HPLC Grade); Method run: 5-95% B, 7 minutes, 0.75 mL/minutes.

WT-Fc Expression, purification, and QC analysis: The IgG1 C_{H}2-C_{H}3 wild-type was synthesized and sub-cloned into an expression vector using known methods. The confirmed plasmid DNA was prepared and used to transfect transiently CHO—S cells. Five to six days after transfection, the cell suspension was centrifuged at 8,000 rpm for 30 minutes to recover the supernatant fraction. The IgG1 Fc wild-type (WT-Fc) was purified by protein A affinity chromatography.

Briefly, the culture supernatant was passed through 0.22 μm filters before loading onto polypropylene columns packed with Protein A high-capacity agarose resin. The resulting flow-through was collected and passed twice more through the column before any unbound protein was washed away with >10 CV (Column Volume) of 1×PBS. The WT-Fc was eluted with 3 ml of 100 mM citrate buffer (pH 3.0) and immediately neutralized with 1 ml of 1 M Tris (pH 8.0). Samples were buffer-exchanged into 1×PBS using Amicon Ultra-4 (Millipore) spin columns with a 10 kDa cutoff, and the purity of purified samples was assessed by 4-20% gradient SDS-PAGE gel. The final Fc protein was approximately 26 kDa as monomeric form. The yield is around 30 to 40 mg per 100 ml culture.

Final cysteine-based conjugation for Zan-PEG₆-Fc-WT: To the purified Fc protein in PBS solution, pH 7.2, was added Compound 13″ (10-12 equiv.) while stirring slowly at 4° C. for 10 minutes. After completion of the addition, the reaction mixture was stirred for 12-72 hours and progress of the reaction was monitored by SDS-PAGE and Matrix Assisted Laser Desorption/Ionization (MALDI) mass analysis.

After completion of reaction, the conjugated crude product was purified using molecular weight cut off (MWCO; 10 kDa, Vivaspin 500, Catalog #GE28-9322-25) filters and centrifugation at 4° C., 15,000/RPM, 10 minute to remove all unreacted linker as well as low molecular weight impurities. The process was repeated 3-5 times (at 5 mg/mL concentration, yield: 60-70%).

The purity and molecular weight of zan-PEG₆-Fc-WT conjugate was confirmed by SDS-PAGE and MALDI analysis, respectively. The final product (zan-Fc-WT) molecular weight was ˜29 kDa, whereas the molecular weight for the Fc-protein alone was 26 kDa.

The above protocol was followed for a second cysteine-based conjugate, zan-Fc-DLE, and the product was confirmed by SDS-PAGE and MALDI analysis. As used herein, the term “zan-Fc-WT” is used interchangeably with the term “Zan-PEG₆-Fc-WT”.

### Example 3

### Comparison of Single Vs. Dual Hapten at 48- and 96-Hpi

A first group of 6-8 weeks old female BALB/c mice (n=5/group) were infected with 10 LD₅₀ of Influenza virus A/Puerto Rico/8/1934 (H1N1) (Catalog No. NR-348, BEI Resources, NIAID, NIH) on day 0 of the experiment. Mice (except the PBS only group) were given an intraperitoneal injection of 8 g/kg human Wig at 24 hpi to achieve humanized titer of anti-DNP and anti-Rhamnose antibodies at the time of test article administration. Mice were treated with test articles at 48 hpi. All the three test articles were administered as a single intranasal dose of 1.5 umol/kg. Mice in the two control groups received PBS as placebo.

For evaluation of drug efficacy, mice were weighed and monitored daily for 14 days post-infection and counted as dead when they lost 25% of their body weight or were diagnosed as moibund.

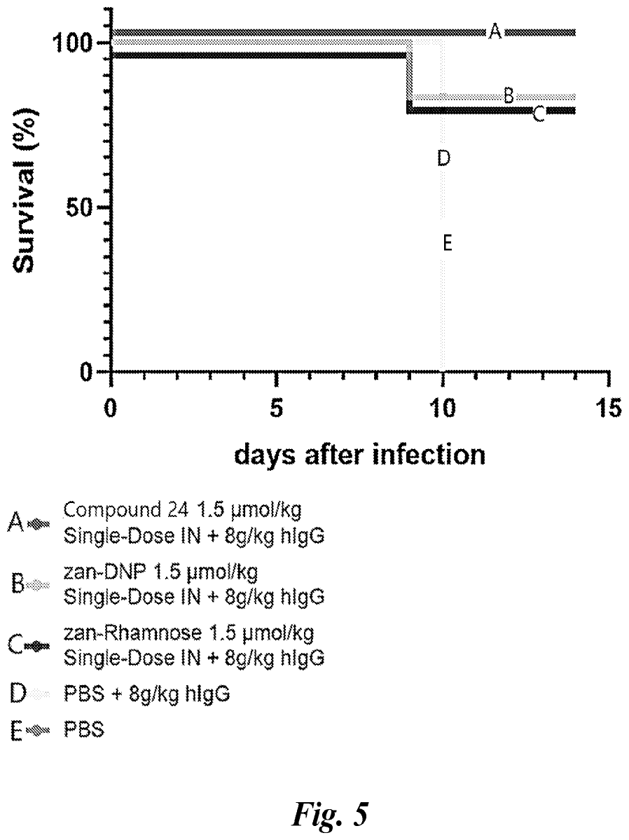

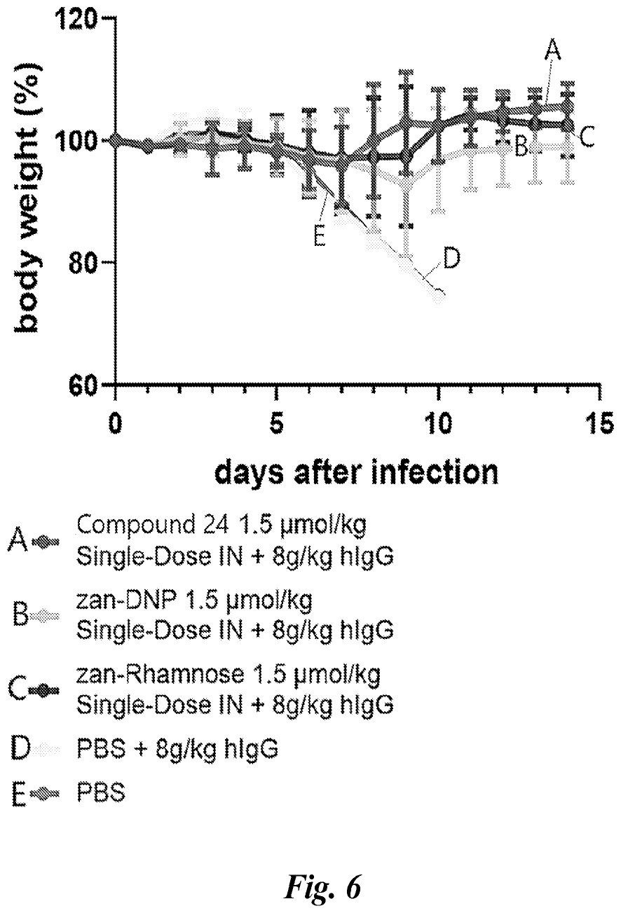

Results of this experiment are depicted in FIG. 5 and FIG. 6. FIG. 5 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMIUNEX®—C) at 24 hpi, and administration of conjugate at 48 hpi. FIG. 6 is a graph of days after infection vs. body weight (%) for the mice treated as described in FIG. 5.

A second group of 6-8 weeks old female BALB/c mice (n=5/group) were infected with 10 LD₅₀ of Influenza virus A/Puerto Rico/8/1934 (H1N1) (Catalog No. NR-348, BEI Resources, NIAID, NIH) on day 0 of the experiment. Mice (except the PBS only group) were given an intraperitoneal injection of s g/kg human IVIg at 72 hpi to achieve humanized titer of anti-DNP and anti-Rhamnose antibodies at the time of test article administration. Mice were treated with test articles at 96 hpi. All the three test articles were administered as a single intranasal dose of 1.5 umol/kg. Mice in the two control groups received PBS as placebo.

As with the previous study, for evaluation of drug efficacy, mice were weighed and monitored daily for 14 days post-infection and counted as dead when they lost 25% of their body weight or were diagnosed as moribund.

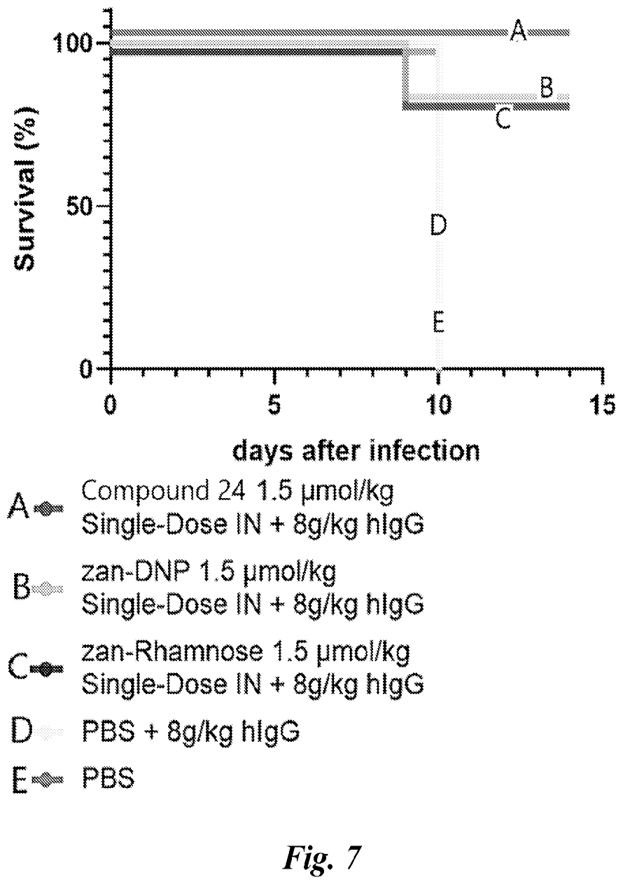

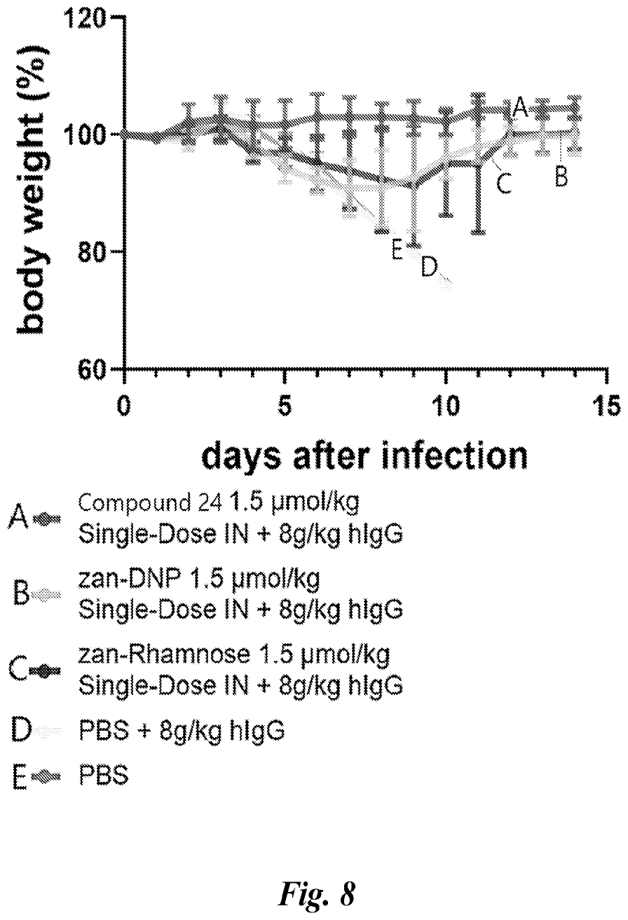

Results of this experiment are depicted in FIG. 7 and FIG. 8. FIG. 7 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX©—C) at 24 hpi, and administration of conjugate at 96 hpi. FIG. 8 is a graph of days after infection vs. body weight (%) for the mice treated as described in FIG. 7.

### Example 4

### Comparison of Dual Hapten with Commercially Available Drugs at 48- and 96-Hours Post-Infection

A first group of 6-8 weeks old female BALB/c mice (n=5/group) were infected with 10 LD₅₀ of Influenza virus A/Puerto Rico/8/1934 (H1N1) (Catalog No. NR-348, BET Resources, NIAID, NIH) on day 0 of the experiment. Mice (except the PBS only group) were given an intraperitoneal injection of 8 g/kg human IVIg at 24 hours hpi to achieve humanized titer of anti-DNP and anti-Rhamnose antibodies at the time of test article administration.

Treatment of the infected mice began at 48 hpi. Zan-DNP-Rhamnose (Compound 24) was administered as a single intranasal dose of 15 umol/kg. Tamiflu was given twice daily for five consecutive days at 5 mg/kg dose and Xofluza was given twice daily for five days at 1.5 mg/kg dose. Mice in the two control groups received PBS as placebo.

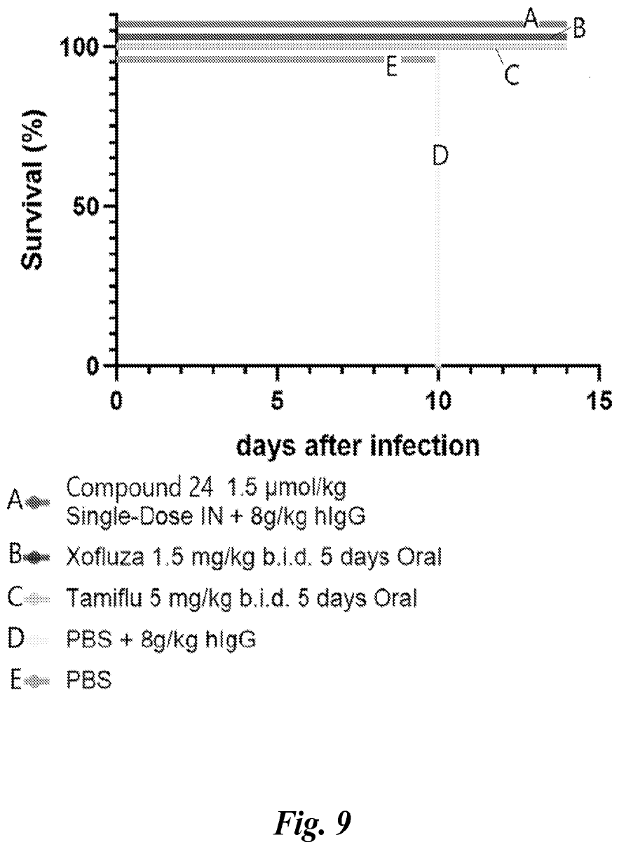

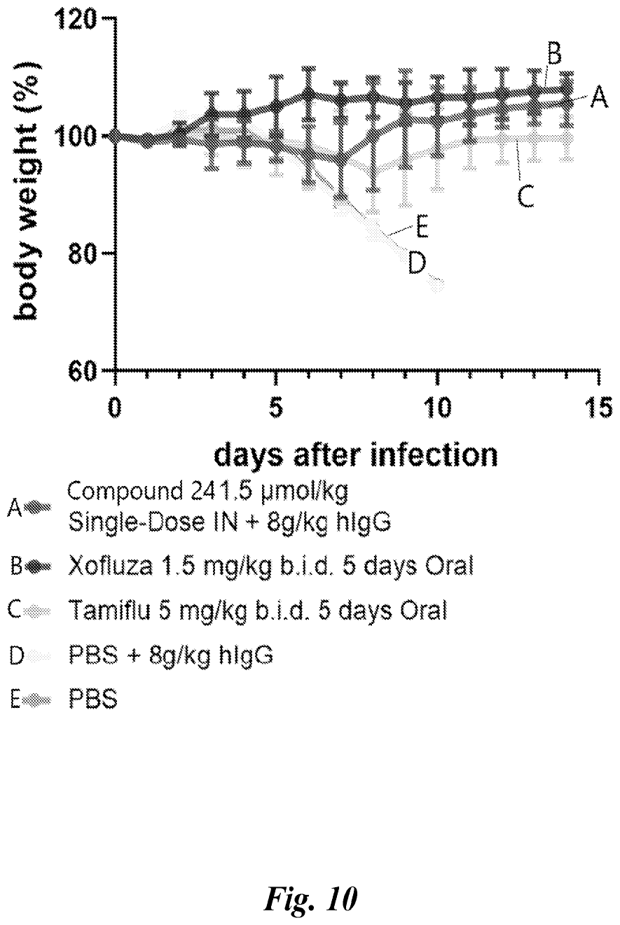

For evaluation of drug efficacy, mice were weighed and monitored daily for 14 days post-infection and counted as dead when they lost 25% of their body weight or were diagnosed as moribund. Results of this experiment are depicted in FIG. 9 and FIG. 10. FIG. 9 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX®—C) at 24 hpi, and administration of conjugate at 48 hpi. FIG. 10 is a graph of days after infection vs. body weight (%) for the mice treated as described in FIG. 9.

Another group of 6-8 weeks old female BALB/c mice (n=5/group) were infected with 10 LD₅₀ of Influenza virus A/Puerto Rico/8/1934 (H1N1) (Catalog No, NR-348, BET Resources, NIAID, NIH) on day 0 of the experiment. Mice (except the PBS only group) were given an intraperitoneal injection of 8 g/kg human IVIg at 72 hpi to achieve humanized titer of anti-DNP and anti-Rhamnose antibodies at the time of test article administration.

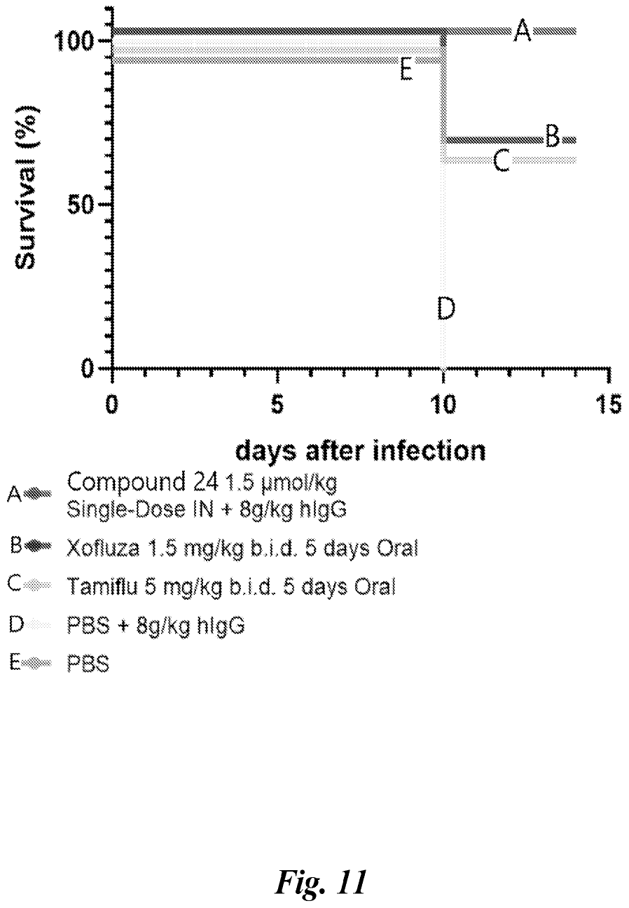

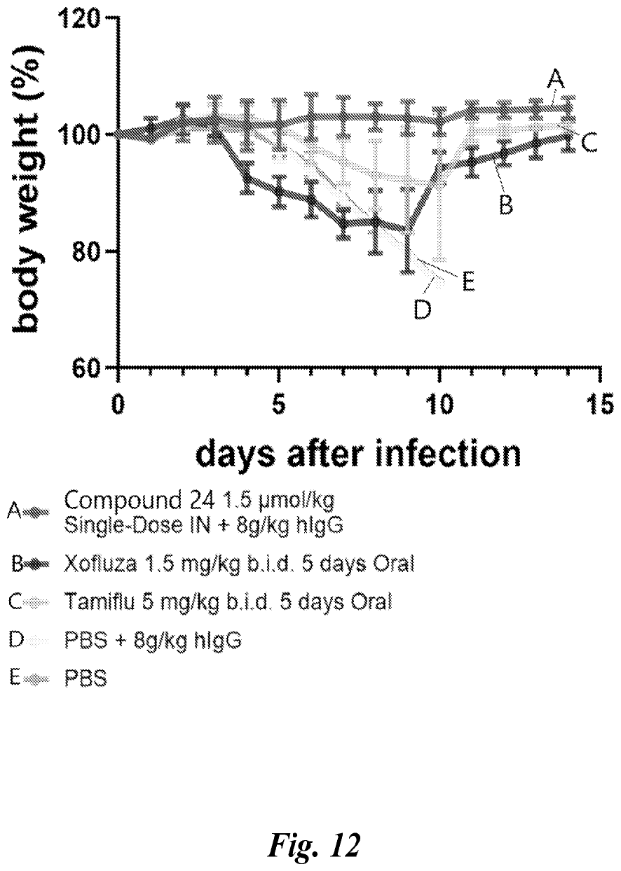

Treatment of the infected mice began at 96 hpi. Zan-DNP-Rhamnose was administered as a single intranasal dose of 1.5 umol/kg. Tamiflu was given twice daily for five consecutive day s at 5 mg/kg dose and Xofluza was given twice daily for five days at 1.5 mg/kg dose. Mice in the two control groups received PBS as placebo. For evaluation of drug efficacy, mice were weighed and monitored daily for 14 days post-infection and counted as dead when they lost 25% of their body weight or were diagnosed as moribund. Results of this experiment are depicted in FIG. 1I and FIG. 12. FIG. 11 is a graph of days after infection vs. survival (%) for mice (n=5/group) infected with 100 LD₅₀ of influenza A H1N1/PR8, removal of anti-influenza antibodies, intraperitoneal administration of human IgG (IVIg (GAMUNEX®—C) at 24 hpi, and administration of conjugate at 96 hpi. FIG. 12 is a graph of days after infection vs. body weight (%) for the mice treated as described in FIG. 11.

### Example 5

### Comparison of Viral Titer of Dual Hapten with Viral Titers of Commercially Available Drugs at 48 Hpi

6-8 weeks old female BALB/c mice (n=5/group) were infected with 10 LD₅₀ of Influenza virus A/Puerto Rico/8/1934 (H1N1) (Catalog No. NR-348, BEI Resources, NIAID, NIH) on day 0 of the experiment. Mice (except the PBS only group) were given an intraperitoneal injection of 8 g/kg human IVIg at 24 hours before test article (TA) administration to achieve humanized titer of anti-DNP and anti-Rhamnose antibodies at the time of test article administration.

Treatment of the infected mice began at either 48 hpi or 96 hpi. Zan-DNP-Rhamnose (ZDR) (Compound 24), zan-DNP (ZD) and zan-Rhamnose (ZR) were administered as a single intranasal dose of 1.5 μmol/kg. Tamiflu was given twice daily for five consecutive days at 5 mg/kg dose and Xofluza was given twice daily for five days at 1.5 mg/kg dose. Mice in the two control groups received PBS as placebo.

To evaluate the rate of reduction in viral titer within 24 hours of TA administration, 2 mice from each cohort were sacrificed by CO₂ asphyxiation at 24 hours post-TA administration and their lungs were harvested and immediately homogenized using gentleMACS Octo Dissociator (Miltenyi Biotec, Bergisch Gladbach, Germany). Viral titers from the lumg homogenates were measured by real time reverse transcription-polymerase chain reaction (RT-PCR). RNA was extracted from the homogenates using Quick-RNA™ Microprep Kit (Zymo Research Corporation, Irvine, California). cDNA synthesis and reverse transcription were performed according to a standard protocol. The primer/probe sets were synthesized to recognize two highly conserved regions of influenza matrix (M) gene. To construct a standard curve for calculation of viral titers, 10-fold dilutions of influenza virus A/Puerto Rico/8/1934 (H1N1) stock solution with a known viral titer were run in parallel with the lung homogenates.

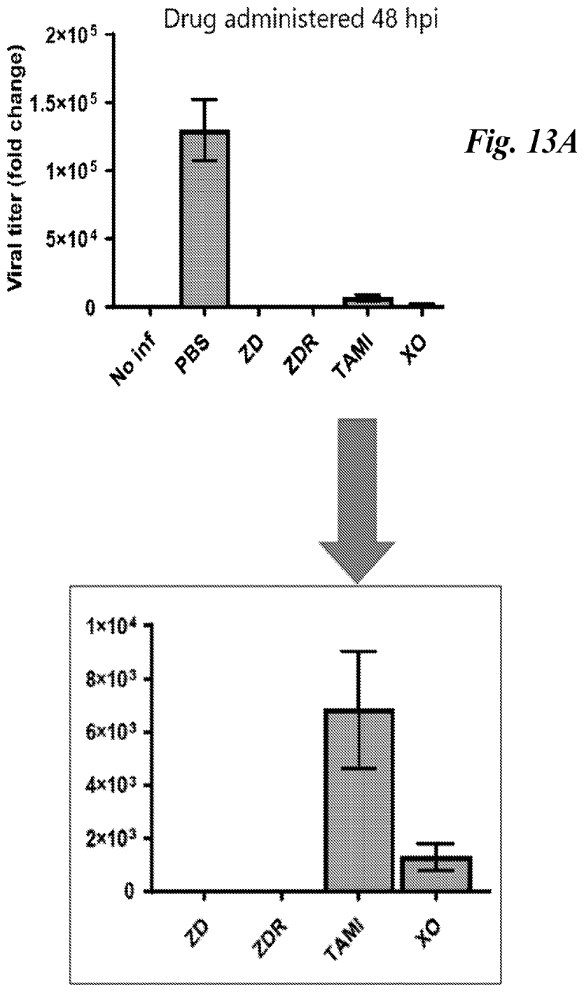

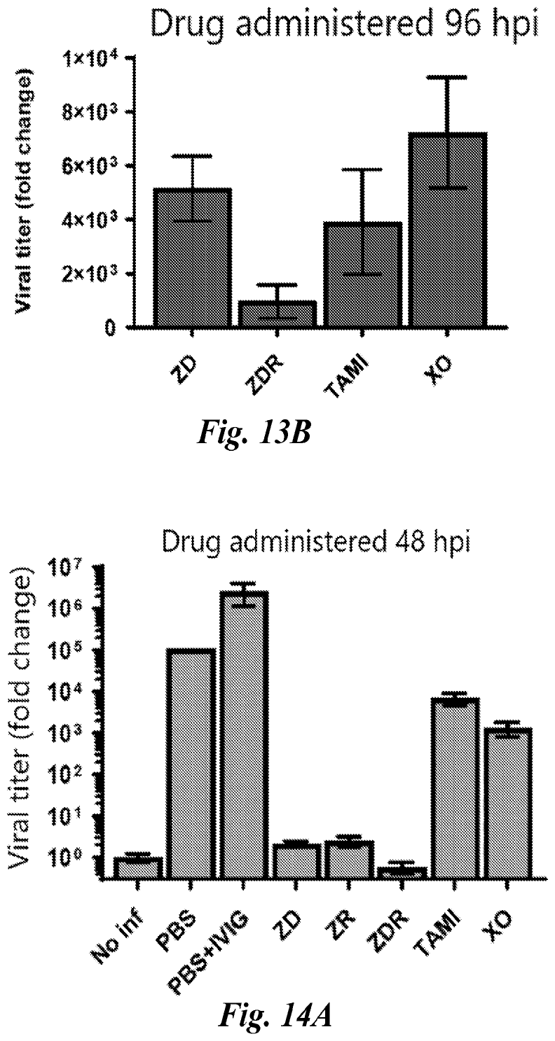

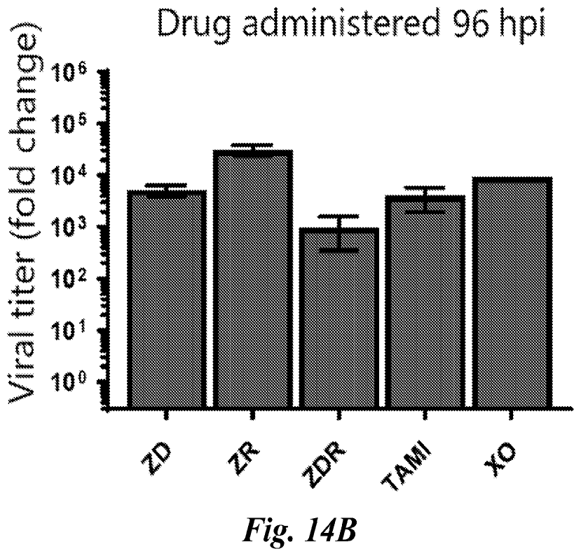

Results of this experiment are depicted in FIGS. 13A, 13B, 14A and 14B. FIG. 13A shows the viral titer (fold change) for the conjugates (Compound 24=ZDR and zan-DNP=ZD) and commercially available drugs (Xofluxa=XO and Tamiflu=TAMI) 48 hpi. FIG. 13B shows the viral titer (fold change) for the conjugates (Compound 24=ZDR and zan-DNP=ZD) and commercially available drugs (Xofluxa=XO and Tamiflu=TAMI) 96 hpi. FIG. 14A shows the viral titer (fold change) for the conjugates (Compound 24=ZDR and zan-DNP=ZD) and commercially available drugs (Xofluxa=XO and Tamiflu=TAMI) 48 hpi. FIG. 14B shows the viral titer (fold change) for the conjugates (Compound 24=ZDR and zan-DNP=ZD) and commercially available drugs (Xofluxa=XO and Tamiflu=TAMI) 96 hpi.

### Example 6

### Oral Administration of Compound 24

6-8 weeks old female Balb/c mice (5 mice/group) were infected with 100×LD₅₀ of Influenza A/H1N1/PR8/1934. Human IVIg was used as the source of anti-hapten antibodies and injected at a dose of 8 g/kg, 24 hours prior to drug administration. The test article, zanamivir-DNP-rhamnose (Compound 24), was administered at 48 hours post-infection with a single dose of 1.5 or 4.5 μmol/kg through intravenous (IV) and oral routes. Mice were weighed and monitored daily for 14 days post-infection and counted as dead when they either lost 25% of their body weight or were diagnosed as moribund.

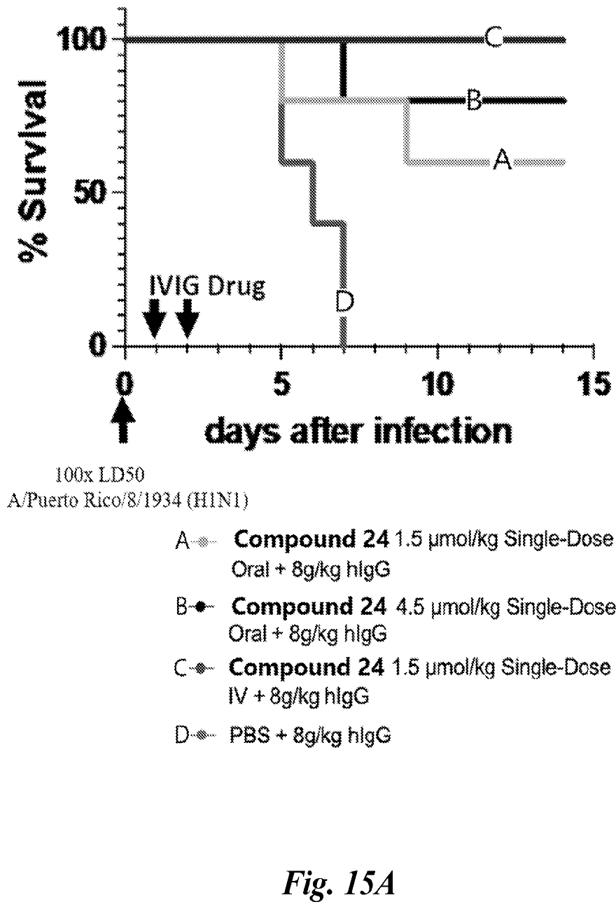

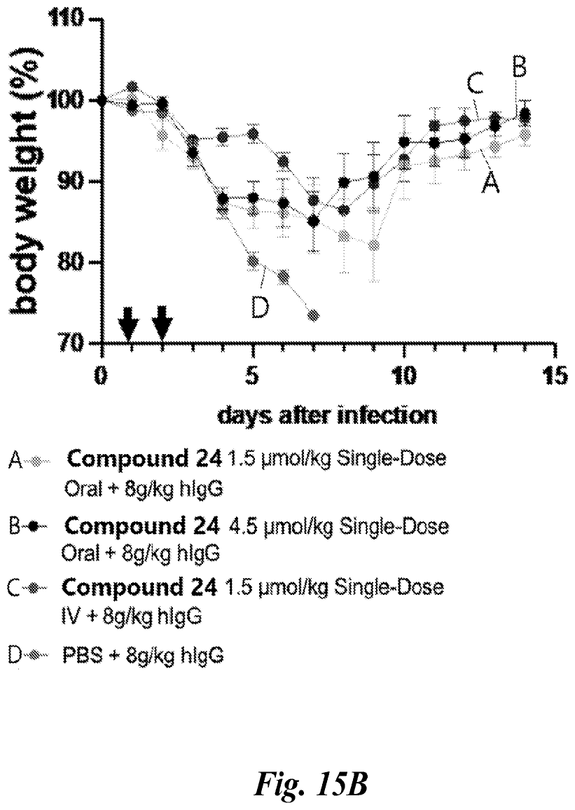

Survival rate dropped to 60% for the same dose of the test article when administered orally, and it increased to 80% with a 3× higher dose. FIG. 15A is a graph of days after infection vs. % survival. FIG. 15B is a graph of days after infection vs. body weight (%).

### Example 7

### Quantitation of Lung Viral Titers

6-8 weeks old female Balb/c mice (2 mice/group) were inoculated intranasally (IN) with 100×MLD₅₀ of influenza A/H1N1/PR8/1934 and then treated 48 hours later with a single IN/IV/OG/SC dose of 1.5 μmol/kg (2.6 mg/kg) or 4.5 μmol/kg (7.8 mg/kg) Compound 24 or PBS. Mice were euthanized 3 days post-infection, and their lungs were harvested and immediately homogenized using gentleMACS Octo Dissociator (Miltenyi Biotec, Bergisch Gladbach, Germany). Viral titers from the lung homogenates were measured by real time RT-PCR. Ribonucleic acid (RNA) was extracted from the homogenates using Quick-RNA™ Microprep Kit (Zymo Research Corporation, Irvine, California). Equal amount of RNA was used for qrt-PCR using One Step PrimeScript™ RT-PCR Kit (Takara Bio, Inc., Kusatsu, Japan) according to manufacturer's protocol. The primer/probe sets were synthesized to recognize two highly conserved regions of influenza matrix (M) gene. To construct a standard curve for calculation of viral titers, 10-fold dilutions of influenza virus A/Puerto Rico/8/1934 (H1N1) stock solution with a known viral titer were run in parallel with the lung homogenates.

Oral administration of Compound 24 in mice did not reduce the viral titer as fast as when administered intranasally or IV with the same or 3× higher dose. Reduced bioavailability resulted in slower reduction of viral titer in the lungs for oral administration compared to IV and IN routes of administration.

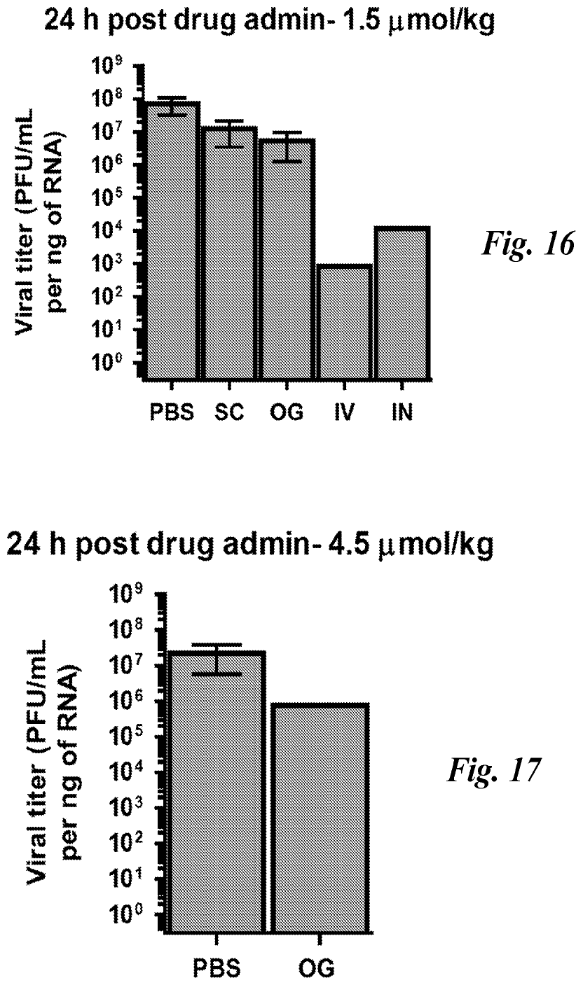

FIG. 16 is a bar graph of route of administration (SC=subcutaneous; OG=oral gavage; IV=intravenous; IN=intranasal; PBS=phosphate-buffered saline) vs. viral titer (PFU/ml per ng of RNA). FIG. 17 is a bar graph of OG and PBS vs. viral titer (PFU/ml per ng of RNA).

### Example 8

### Pharmacokinetics of Oral Compound 24

6-8 weeks old female Balb/c mice (3 mice/group) were administered with a single intravascular (IV) or oral dose of 1.5 μmol/kg (2.6 mg·kg), 1.5 μmol/kg (2.6 mg/kg), or 13.5 μmol/kg (23.4 mg/kg) of Compound 24 and blood samples were collected at 5 minutes, 15 minutes, 30 minutes, 1 hour, 2 hours, 4 hours, 6 hours, 8 hours and 12 hours post-drug administration. The plasma concentration of Compound 24 was determined by liquid chromatography with tandem mass spectrometry (LC-MS/MS). IVIg was administered as the source of anti-hapten antibodies 24 hours prior to drug administration.

Oral administration resulted in a significantly lower Cₘₐₓ (maximum concentration in the plasma) compared to IV, even at a 9× higher dose, but the half-life (t_{1/2}) increased from approximately 0.4 hours to 4 hours with a 9× higher dose.

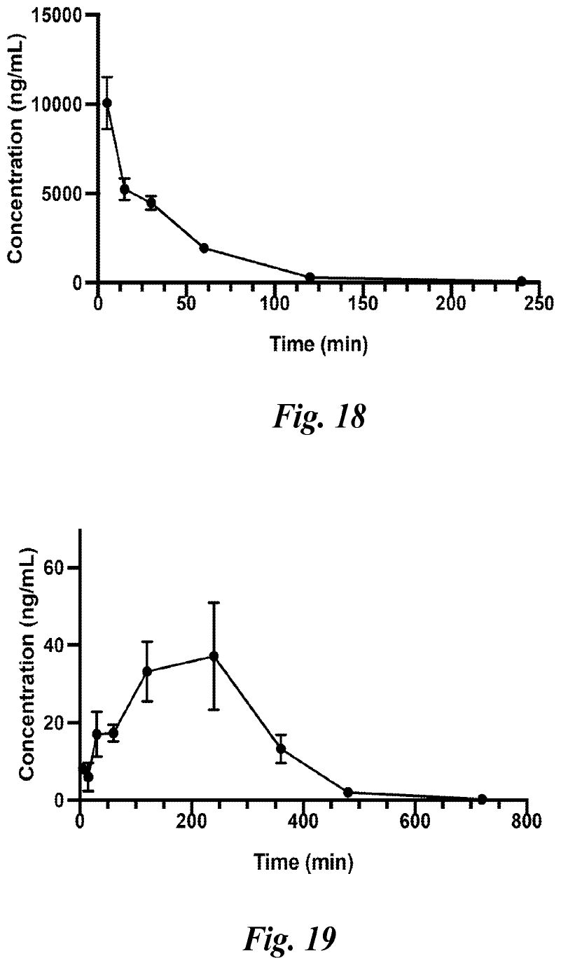

FIG. 18 is a graph of time (h=hours) vs. concentration (ng/mL) for IV administration of a single dose of 1.5 mol/kg (n=3 mice/timepoint). FIG. 19 is a graph of time (h=hours) vs. concentration (ng/mL) for oral administration of a single dose of 13.5 gmol/kg (n=3 mice/timepoint).

### Example 9

### Virus Challenge and Mouse Therapy Studies of Increasing the Oral Dosing Frequency

6-8 weeks old female Balb/c mice (5 mice/group) were infected with 100×LD₅₀ of Influenza A/H1N1/PR8/1934. Human IVIg was used as the source of anti-hapten antibodies and injected at a dose of 8 g/kg at 24 hours prior to drug administration. The test article, Compound 24, was administered at 48 hpi with a single dose of 1.5 μmol/kg zan-DNP-Rhamnose (IV) or two doses of 4.5 umol/kg zan-DNP-rhamnose (oral). Mice were weighed and monitored daily for 14 days post-infection and counted as dead when they either lost 25% of their body weight or were diagnosed as moribund.

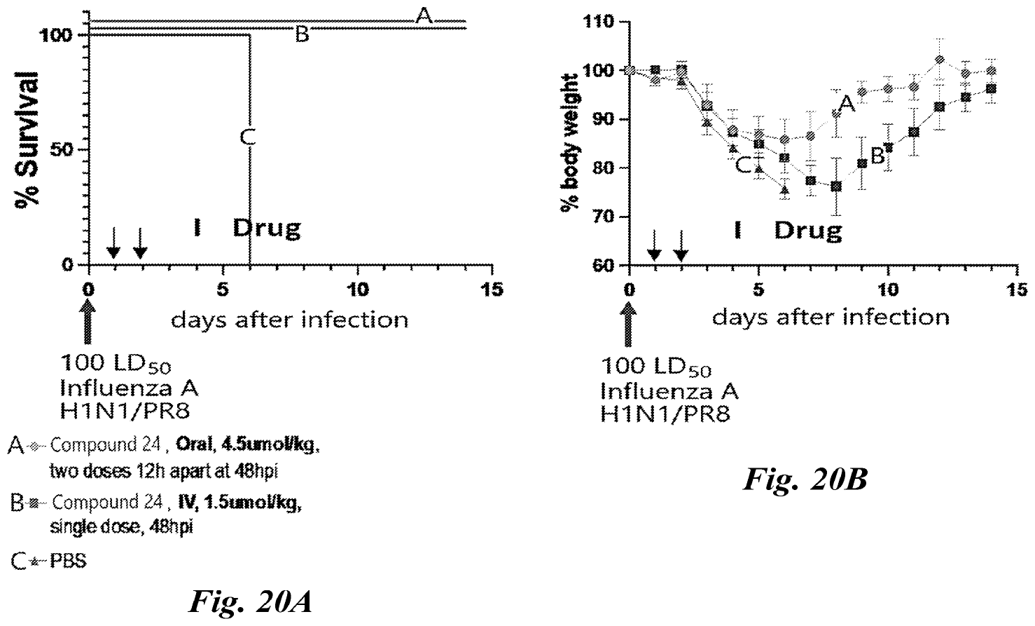

Increasing the dosing frequency to two doses 12-hours apart provided 100% survival (as compared to 80% survival with a single dose of 4.5 mol/kg zan-DNP administered orally). FIG. 20A is a graph of days after infection vs. % survival. FIG. 20B is a graph of days after infection vs. % body weight.

### Example 10

### Quantitation of Lung Viral Titers

6-8 weeks old female Balb/c mice (2 mice/group) were inoculated IN with 100×MLD₅₀ of Influenza A/H1N1/PR8/1934 and then treated 48 hours later with a single IV dose of 1.5 μmol/kg (2.6 mg/kg) or two oral doses (12 hours apart) of 4.5 μmol/kg (7.8 mg/kg) Compound 24 or PBS. Mice were euthanized at 3-, 5- and 8-days post-infection, and their lungs were harvested and immediately snap frozen in RNA lysis buffer. Lung homogenates were prepared using gentleMACS Octo Dissociator (Miltenyi Biotec, Bergisch Gladbach, Germany). Viral titers from the lung homogenates were measured by qrt-PCR. RNA was extracted from the homogenates using Quick-RNA™ Microprep Kit (Zymo Research Corporation, Irvine, California). Equal amount of RNA was used for qrt-PCR using One Step PrimeScript™ RT-PCR Kit (Takara Bio, Inc., Kusatsu. Japan) according to manufacturer's protocol. The primer/probe sets were synthesized to recognize two highly conserved regions of influenza matrix (M) gene. To construct a standard curve for calculation of viral titers, 10-fold dilutions of influenza virus A/Puerto Rico/8/1934 (H1N1) stock solution with a known viral titer were run in parallel with the lung homogenates.

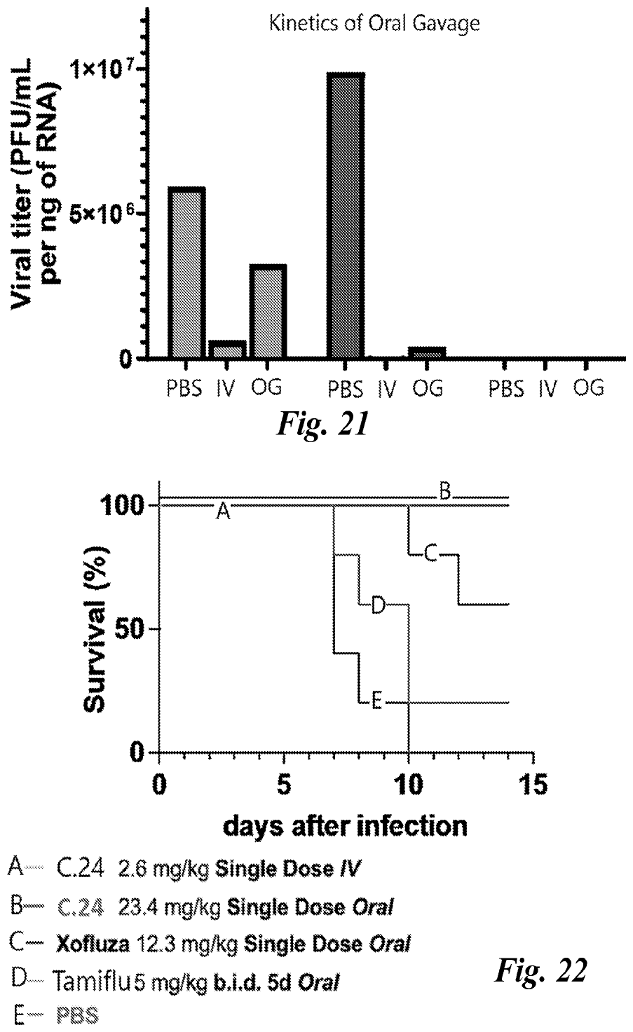

Oral administration resulted in a slower rate of viral titer reduction than IV administration but was able to eradicate completely the viral titer by day 8 post-infection. Both IV and oral administration of Compound 24 can fully eliminate viral infection from the lungs, with faster action through the IV route than the oral route. FIG. 21 is a graph of days post-infection for PBS and IV and oral (OG=oral gavage) administration of zanamivir-DNP-rhamnose vs. viral titer (PFU/ml per ng of RNA).

### Example 11

### Efficacy Comparison of Compound 24 with the Standard of Care (SOCs) (96 Hpi Treatment)

6-8 weeks old female Balb/c mice (5 mice/group) were infected with 10×LD₅₀ of Influenza A/H1N1/PR8/1934 (day 0). A control group of healthy mice (not infected at day 0) were also tested as a control.

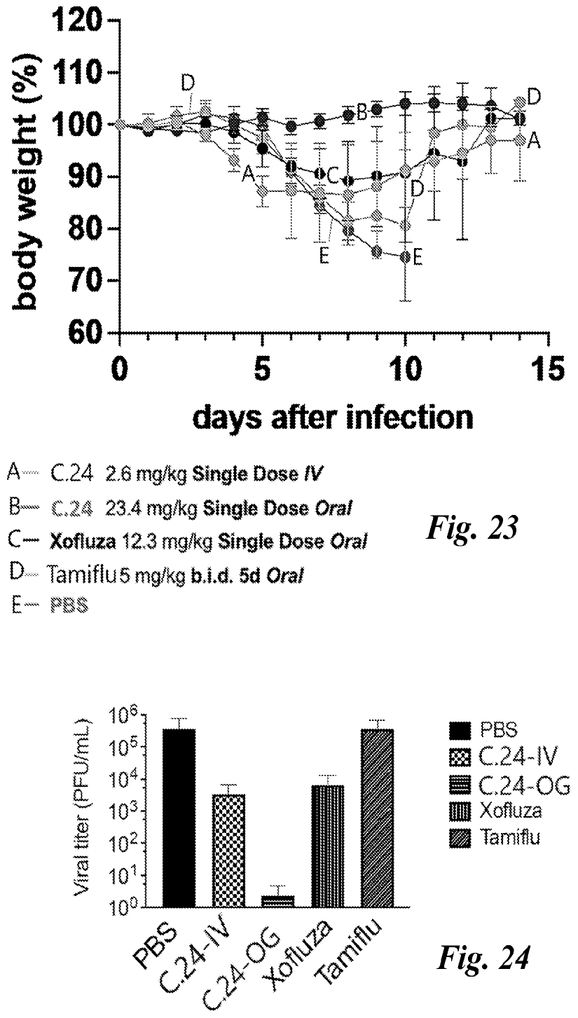

Human IVIg was used as the source of anti-hapten antibodies and injected at an optimized dose of 6 g/kg, 24 hours prior to drug administration to each cohort. The test articles and the SOCs were administered at 96 hpi with a single IV dose of 2.6 mg/kg of Compound 24 (C.24 (A) in FIGS. 22 and 23), a single oral dose of 23.4 mg/kg of Compound 24 (C.25 (B) in FIGS. 22 and 23), a single oral dose of 12.3 mg/kg baloxavir marboxil (Xofluza®) (Xofluza (C) in FIGS. 22 and 23), five days of twice daily oral administration of 5 mg/kg oseltamivir phosphate (Tamiflu©) (Tamiflu (D) in FIGS. 22 and 23), or PBS (E in FIGS. 22 and 23) (healthy mice data not shown in FIGS. 22 and 23). Mice were weighed and monitored daily for 14 days post-infection and counted as dead when they either lost 25% of their body weight or were diagnosed as moribund.

Both single dose IV (A) and oral administration of Compound 24 (B) resulted in 100% survival of virus-infected mice that was not achievable by either of the two SOC drugs tested. Compound 24 outperformed the current standard of care antivirals when treated at 96 hpi. FIG. 22 is a graph of days after infection vs. survival (%). FIG. 23 is a graph of days after infection vs. body weight (%). (Compound 24=C.24 in FIGS. 23 and 24).

24 hours after drug administration (120 hpi), mice were subjected to isoflurane anesthesia followed by cervical dislocation. Lung tissues were fixed in 10% neutral buffered formalin (NBF), embedded in Paraffin, sectioned, and stained with Hematoxylin and Eosin (H&E) and then imaged under Nikon Eclipse light microscope at 10× magnification. Qualitative histological analysis of the lung was performed, with images shown in FIG. 33. The virus-infected mice that did not receive treatment exhibited signs of diffuse alveolar damage, pulmonary edema, and excessive infiltration of inflammatory cells, whereas mice treated with Compound 24 either intravenously or orally exhibited a significant decrease in virus-induced histopathological changes in the lung tissue as compared to the no-treatment group.

In parallel, two cohorts of healthy mice were injected with Compound 24 IV and Compound 24 OG respectively and another cohort of mice received no intervention. 24 hours after drug administration, mice were subjected to isoflurane anesthesia followed by cervical dislocation. Lung, kidney, liver, stomach, and small intestinal tissues were fixed in 10% neutral buffered formalin (NBF), embedded in Paraffin, sectioned, and stained with H&E (except kidney, kidney tissues were stained with periodic acid Schiff (PAS)) and then imaged under Nikon Eclipse light microscope at 10× magnification. Qualitative histological analysis of the lung was performed, with images shown in FIG. 33. No abnormal changes were observed in tissue morphology in the mice injected with Compound 24 as compared to the no intervention group.

### Example 12

### Efficacy Comparison of Compound 24 with Mono-Haptens (96 Hpi Treatment)

A targeted therapeutic strategy with a dual mechanism of action that elicits host immune response against the virus and virus-infected cells is disclosed. The neuraminidase inhibitor zanamivir is deployed (as a fragment) as a targeting ligand. Neuraminidase appears both on the influenza viral envelope and the infected cell surface. The conjugate comprising the zanamivir fragment is bound to haptens that bind to naturally occurring antibodies in humans. Once recruited, these anti-hapten antibodies bind and activate innate immune system against the virus and virus-infected cells.

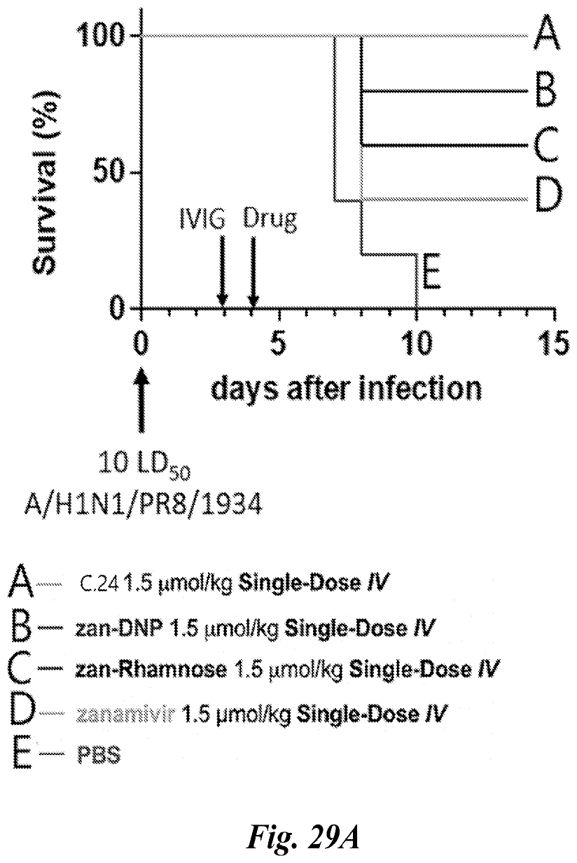

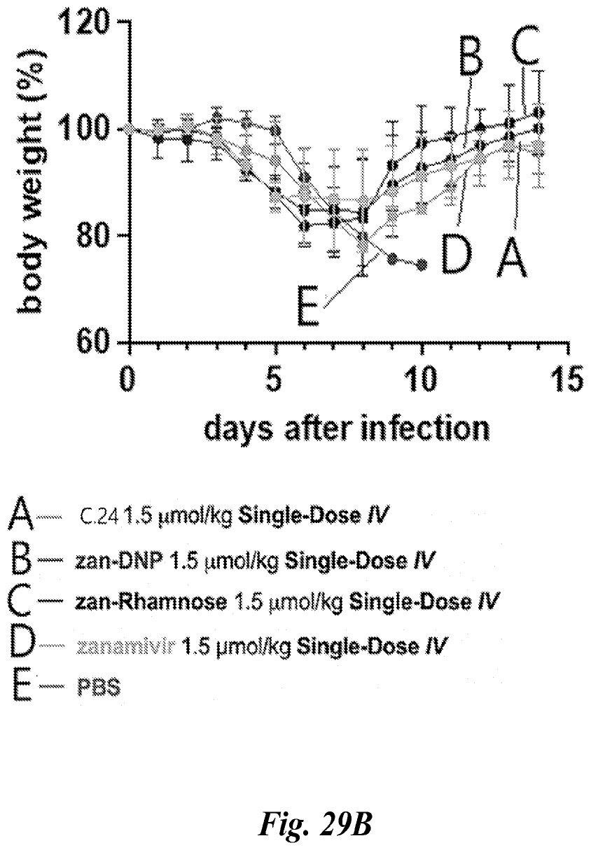

6-8 weeks old female Balb/c mice (5 mice/group) were infected with 10×LD₅₀ of Influenza A/H1N1/PR8/1934 (day 0). Human IVIg was used as the source of anti-hapten antibodies and injected at an optimized dose of 6 g/kg, 24 hours prior to drug administration. The test articles were administered at 96 hpi with a single IV dose of 1.5 μmol/kg of: (a) Compound 24 ((A) in FIGS. 29A and 29B); (b) zanamivir-DNP (mono-hapten conjugate; (B) in FIGS. 29A and 29B); (c) zanamivir-Rhamnose (mono-hapten conjugate; (C) in FIGS. 29A and 29B); (d) zanamivir ((D) in FIGS. 29A and 29B); or (e) PBS (control) ((E) in FIGS. 29A and 29B). Mice were weighed and monitored daily for 14 days post-infection and counted as dead when they either lost 25% of their body weight or were diagnosed as moribund.

When tested in BALB/c mice supplemented with IVIg and infected with influenza A virus (H1N1, A/Puerto Rico/8/1934), the zanamivir-dual hapten conjugate (Compound 24) demonstrated superior antiviral activity than mono-hapten conjugates at both early and late-stage infection. Further, the dual hapten conjugate showed better activity in late-stage infection in comparison to the mono-hapten conjugates at a single dose. Accordingly, Compound 24 may treat both early and late-stage influenza infection.

### Example 13

### 24-Hour Lung Titer Comparison

6-8 weeks old female Balb/c mice (5 mice/group) were infected with 10×LD₅₀ of Influenza A/H1N1/PR8/1934 and, 48 hours later, treated with: (a) a single IV dose of 1.5 μmol/kg of Compound 24; (b) a single OG dose of 13.5 gmol/kg of Compound 24; (c) a single OG dose of 5 mg/kg Tamiflu©; (d) a single OG dose of 10 mg/kg Xofluza®; or (e) a single IV dose of 100 μL PBS.

Twenty-four hours after treatment, viral titers from the lung homogenates were measured by real time RT-PCR with the results set forth in FIG. 24 showing titers lowest for Compound 24 delivered orally (Compound 24=C.24).

### Example 14

### Compound 24 Route Administration Comparison

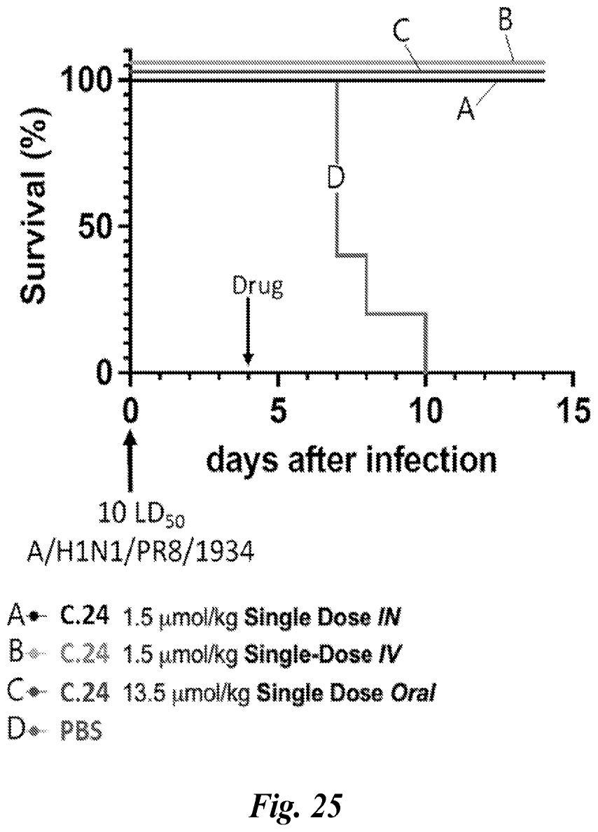

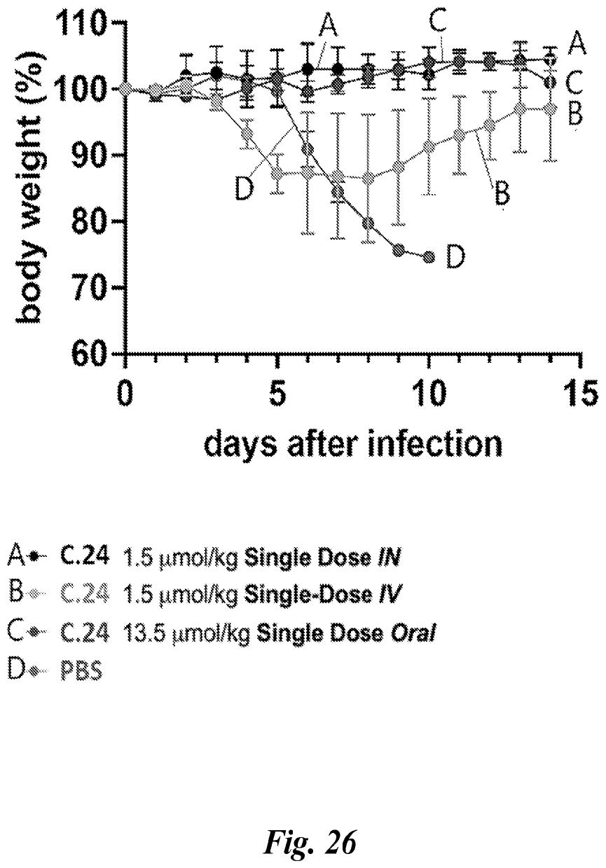

Balb/c mice were exposed to infection at day 0 as indicated in FIG. 25. Four days after infection challenge, Compound 24 was administered by three different routes as indicated—intranasal, IV, and oral, with oral being the highest dose at 13.5 gmol/kg, the other does being 1.5 gmol/kg. Results show that survival was 100% after 14 days for all three Compound 24 cohorts whereas mice treated with PBS did not survive past day 10. An infection/% body weight graph is presented in FIG. 26.

### Example 15

### Antibody-Mediated Effector Functions In Vitro

To test the proof of concept of the mechanism of action of Compound 24 in vitro, Antibody Dependent Cellular Cytotoxicity (ADCC) and Complement Dependent Cytotoxicity (CDC) assays were performed. The ADCC assay was performed using an ADCC kit (Promega, cat. no. G7010; Promega Corporation, Madison, WI) with influenza viral neuraminidase (N1)-transfected (NA-HEK) and wild-type (WT) Human Embryonic Kidney (HEK 293) cells. For this purpose, cells were plated in triplicate (100 μl, 5000 cells/well) in 96-well black walled plates (Corning Life Sciences, Corning, NY) and then treated with serial dilutions of Compound 24 in the presence or absence of 100-fold excess zanamivir. After incubating at 37° C. for 2 hours, human IVIg was added to each well and the plates were incubated at 37° C. for another 30 minutes. Finally, ADCC effector cells were added at 75,000 cells/well and incubated overnight at 37° C. under 5% CO₂.

The amount of firefly luciferase produced by ADCC effector cells was then quantified using Bio-Glo™ Luciferase Assay Reagent (included in the kit). Luminescence was measure using Synergy Neo2 HTS Multi-Mode Microplate Reader (BioTek Instruments, Winooski, VT).

Analysis of virus-infected cell killing by the complement system was conducted by a complement dependent cytotoxicity (CDC) assay. N1-transfected (NA-HEK) and wild-type (WT) HEK 293 cells were harvested and plated in triplicate (100 μl, 5000 cells/well) in 96-well black walled plates (Corning Life Sciences, Corning, NY) and then treated with serial dilutions of Compound 24 in the presence or absence of 100-fold excess of zanamivir. (Zanamivir is the free drug that competes with Compound 24 when used in excess). After incubating at 37° C. for 2 hours, human IVig and human serum were added to each well and the plates were incubated overnight at 37° C. under 5% CO₂. Cell viability was measured using CellTiter 96® Aqueous Non-Radioactive Cell Proliferation Assay (Promega Corporation, Madison, WI).

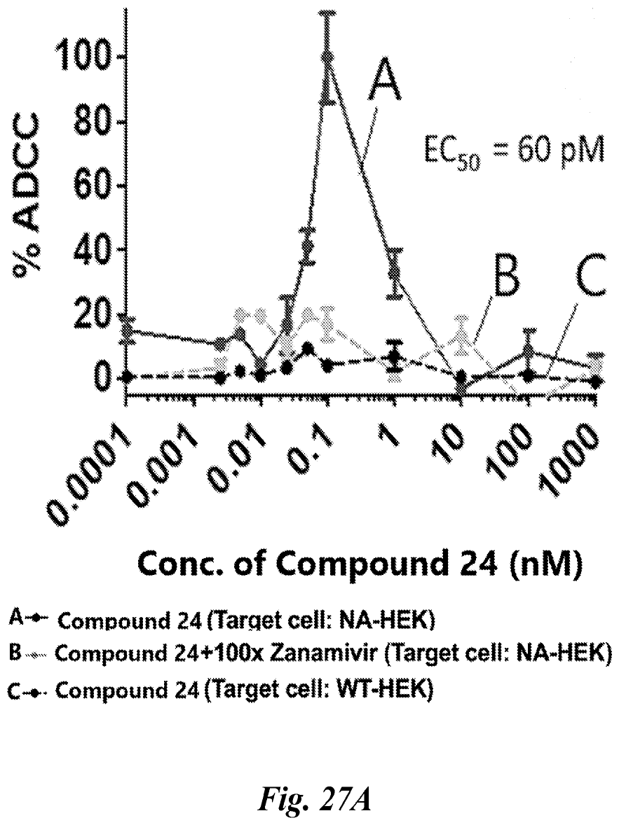

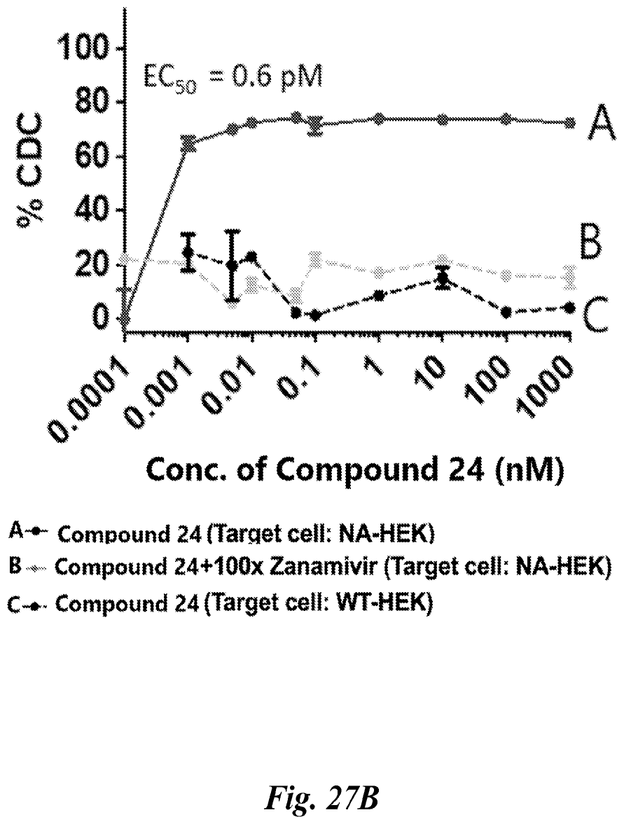

Compound 24 induced antibody-mediated effector functions in vitro with high potency and selectivity by interacting with the Fc-receptors expressed on the effector cells and with the complement protein in the presence of antibodies. FIGS. 27A and 27B show graphs of the concentration of Compound 24 (in nM) versus % ADCC and % CDC, respectively.

### Example 16

### Dose Escalation of IV Administration of Compound 24

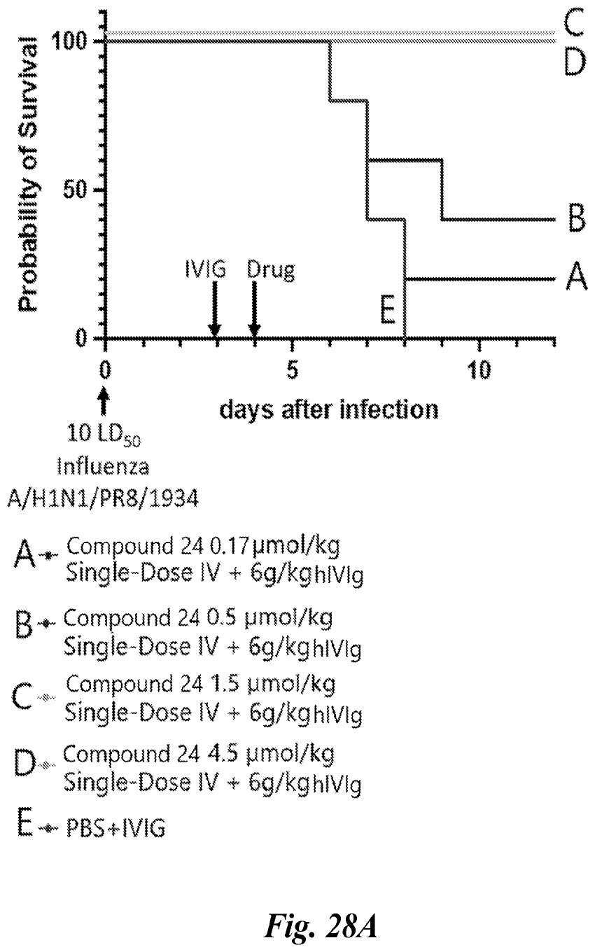

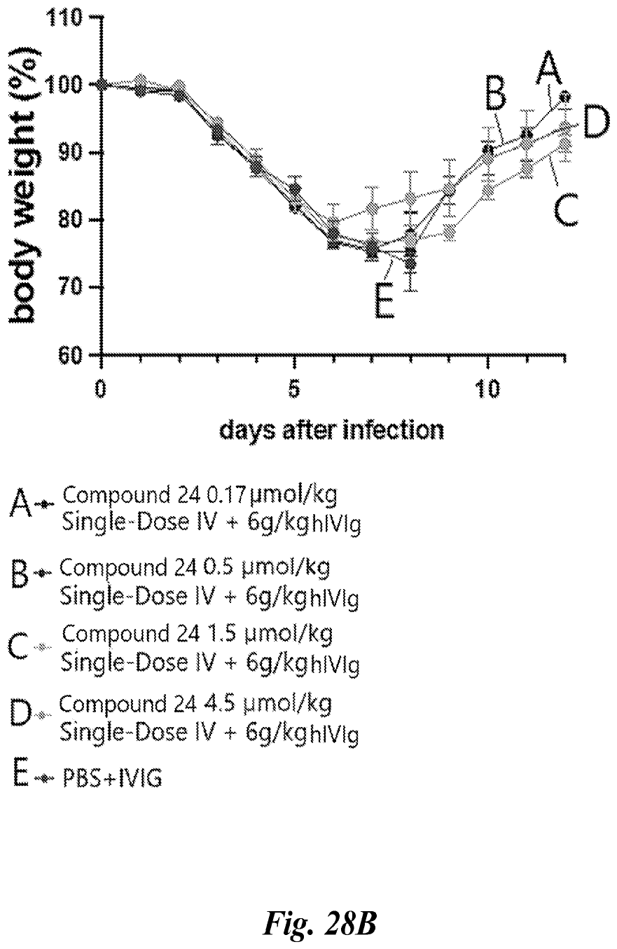

6-8 weeks old female Balb/c mice (5 mice/group) were infected with 10×LD₅₀ of Influenza A/H1N1/PR8/1934 (day 0). Human IVIg was used as the source of anti-hapten antibodies and injected at an optimized dose of 6 g/kg, 24 hours prior to drug administration. Each test group of mice were administered at 96 hpi with a single IV dose of: (a) 0.17 μmol/kg of Compound 24 ((A) in FIGS. 28A and 28B); (b) 0.5 μmol/kg of Compound 24 ((B) in FIGS. 28A and 28B); (C) 1.5 μmol/kg of Compound 24 ((C) in FIGS. 28A and 28B); (d) 4.5 mol/kg of Compound 24 ((D) in FIGS. 28A and 28B); and (e) PBS (E) in FIGS. 28A and 28B). Mice were weighed and monitored daily for 14 days post-infection and counted as dead when they either lost 25% of their body weight or were diagnosed as moribund.

Both single dose IV of 1.5 μmol/kg of Compound 24 (C) and single dose IV of 4.5 μmol/kg of Compound 24 (D) resulted in 100% survival of mice that was not achievable by the other doses tested or the control (PBS). FIGS. 28A and 28B show graphs of days after infection vs. survival (%), and days after infection vs. body weight (%), respectively.

### Example 17

### Efficacy Against Multiple Flu Strains

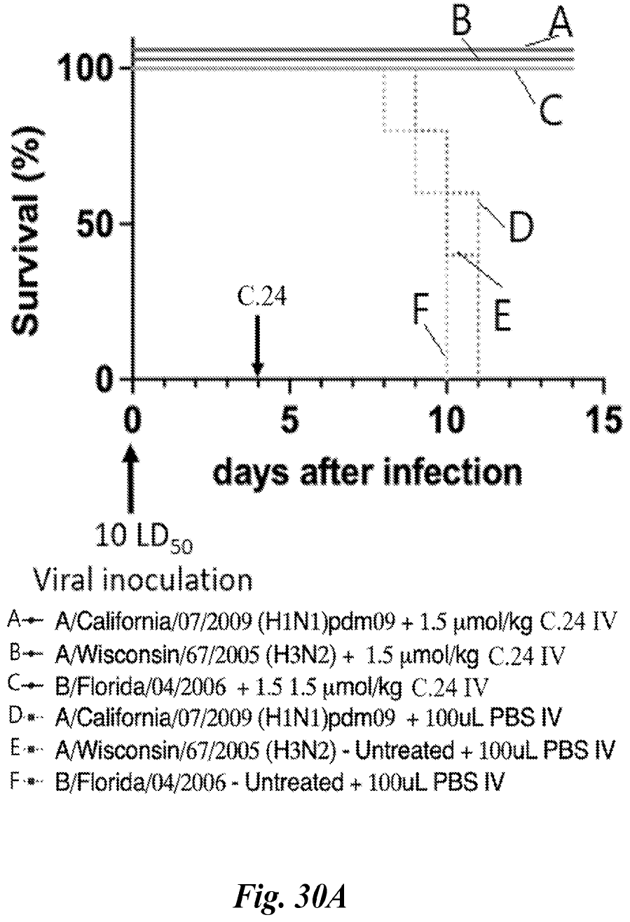

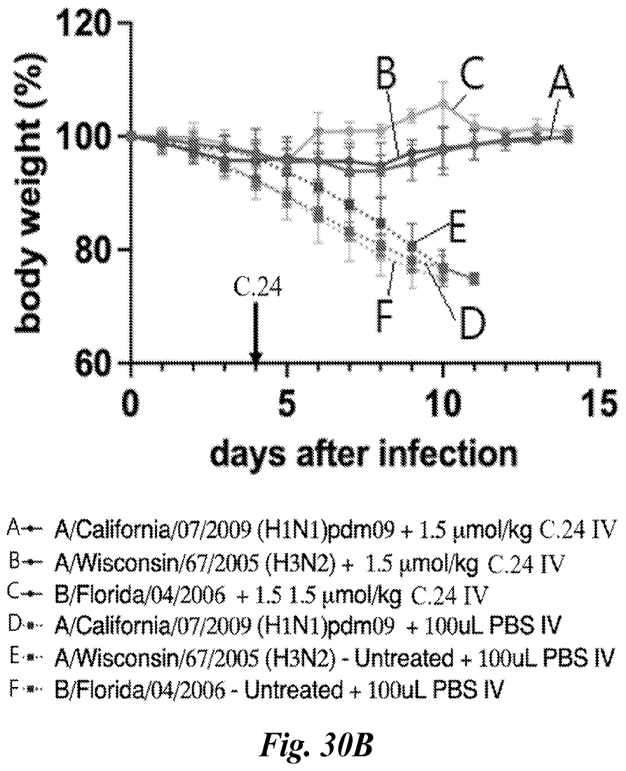

At day 0, 6-8 weeks old female Balb/c mice (5 mice/group) were infected with 10×LD₅₀ of Influenza A/Califomia/07/2009 (H1N1)pdm09, Influenza A/Wisconsin/67/2005, or Influenza B/Florida/04/2006. The mice infected with Influenza A/California/07/2009 (H1N1)pdm09 were administered at 96 hpi with a single IV dose of either 1.5 μmol/kg of Compound 24 ((A) in FIGS. 30A and 30B) or PBS ((D) in FIGS. 30A and 30B). The mice infected with Influenza A/Wisconsin/67/2005 were administered at 96 hpi with a single IV dose of either 1.5 μmol/kg of Compound 24 ((B) in FIGS. 30A and 30B) or PBS ((E) in FIGS. 30A and 30B). The mice infected with Influenza B/Florida/04/2006 were administered at 96 hpi with a single IV dose of either 1.5 gmol/kg of Compound 24 ((C) in FIGS. 30A and 30B) or PBS ((F) in FIGS. 30A and 30B).

Compound 24 was equally potent against all strains of flu tested and survival was 100% after 14 days for all three Compound 24 cohorts whereas mice treated with PBS did not survive past day 11.

### Example 18

### Inflammatory Cytokine and Chemokine Levels

6-8 weeks old female Balb/c mice (5 mice/group) were infected with 10×LD₅₀ of Influenza A/H1N1/PR8/1934 (day 0). Each test group of mice were administered a single dose of one of the following treatments at 48 hpi or 96 hpi-100 μL PBS via IV (A), 2.6 mg/kg Compound 24 via single dose IV (B); 23.4 mg/kg Compound 24 via single dose OG (C), 12.3 mg/kg Xofluza© via single dose OG (D), and 5 mg/kg Tamiflu© via OG, b.i.d. over 5 days (E). Two groups of mice were not infected with the virus but administered Compound 24 via IV at 96 hpi (F) (with respect to the other groups) and a third group of mice were neither infected nor received any treatment (G). The cytokines and chemokines from each group were measured in lung tissue samples via BioLegend's LEGENDplex™ bead-based immunoassay was used to determine the cytokine and chemokine levels using standard protocol provided by the manufacturer (BioLegend, San Diego, CA).

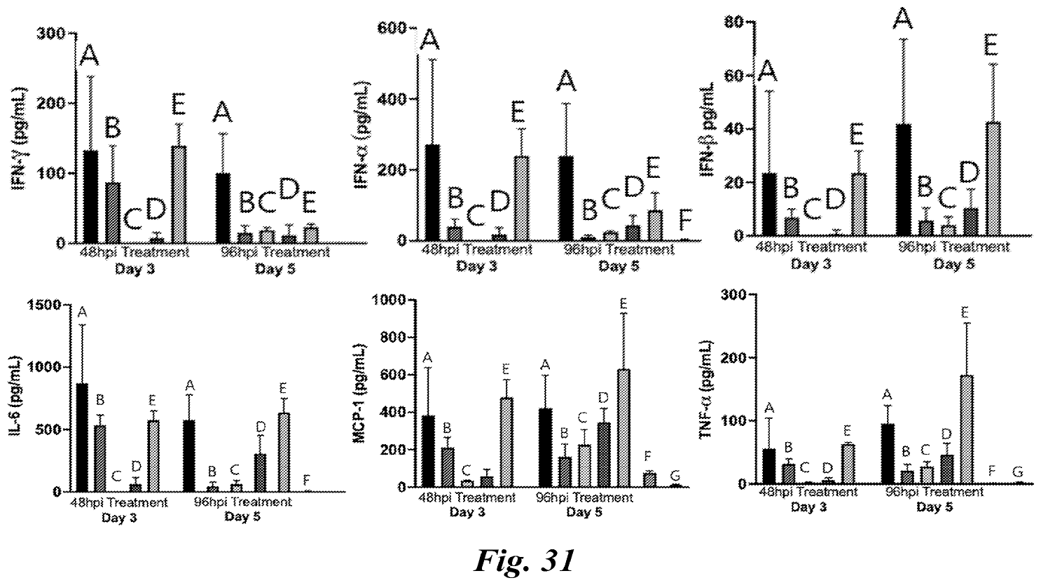

FIG. 31 shows graphs of specific cytokine and chemokine expression levels in the lung measured for each group at both the 48 hpi and 96 hpi treatment groups. Where no cytokines or chemokine levels are shown for a control group in a graph, there were no detectable levels. Compound 24, irrespective of if administered orally or intravascularly, did not induce a cytokine storm in the lung in either case, but rather prevented its occurrence by rapidly eliminating the root cause of cytokine storm i.e., the viral infection. These data support administration of Compound 24 can prevent lung inflammation and injury initiated by an Influenza viral infection.

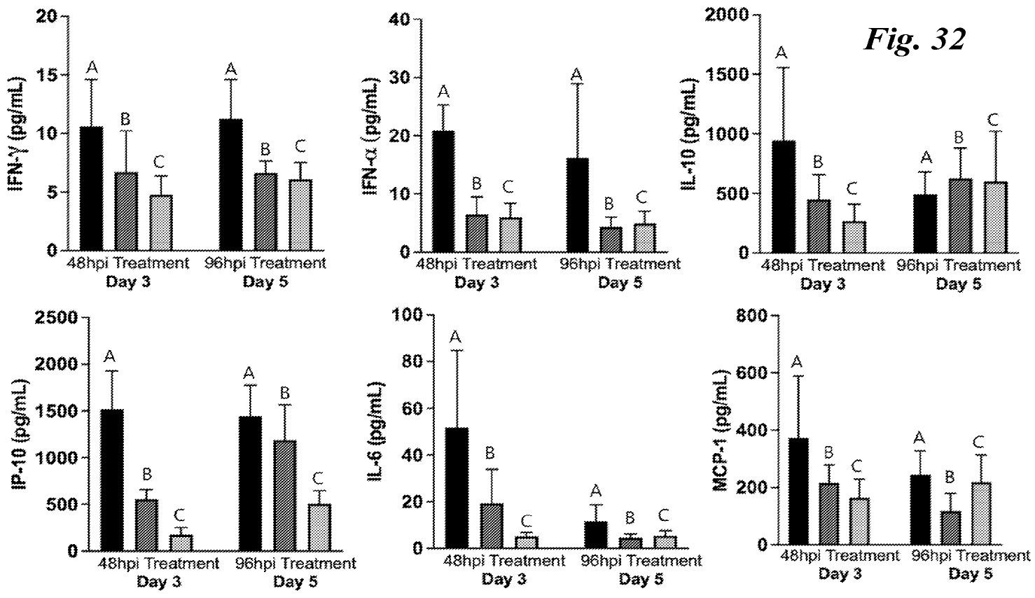

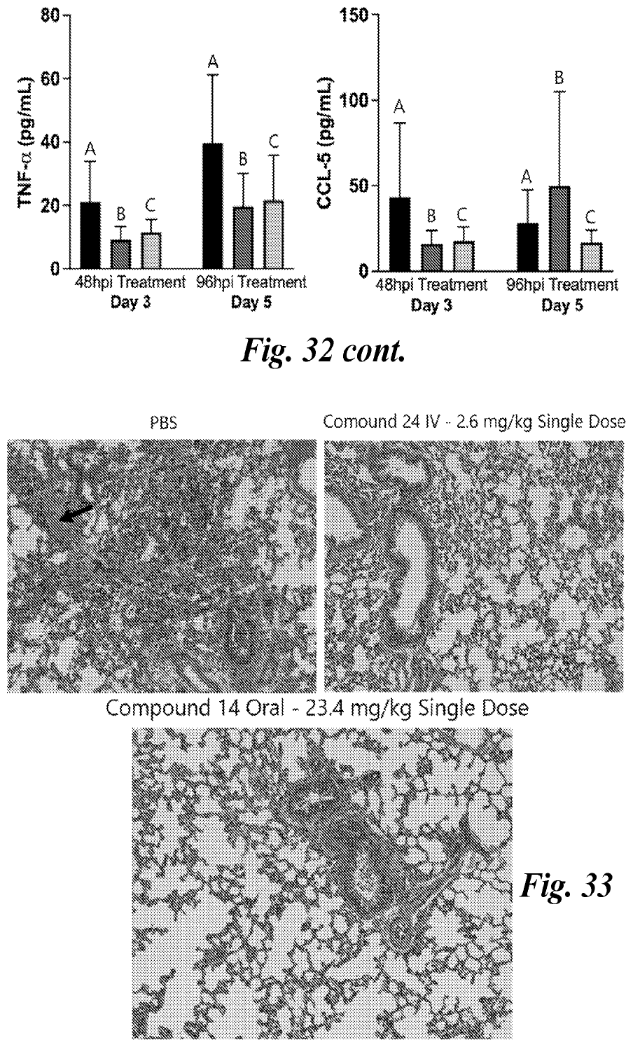

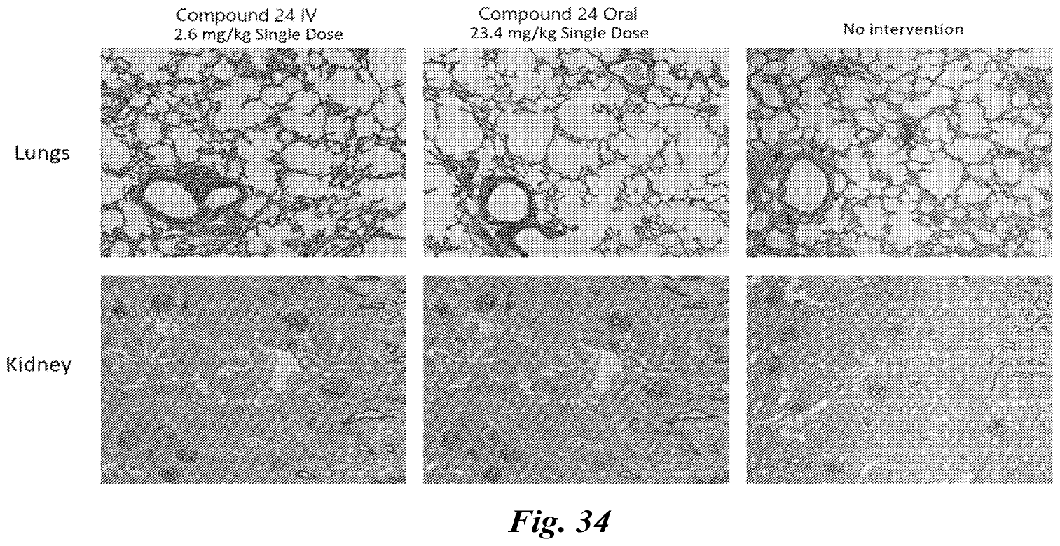

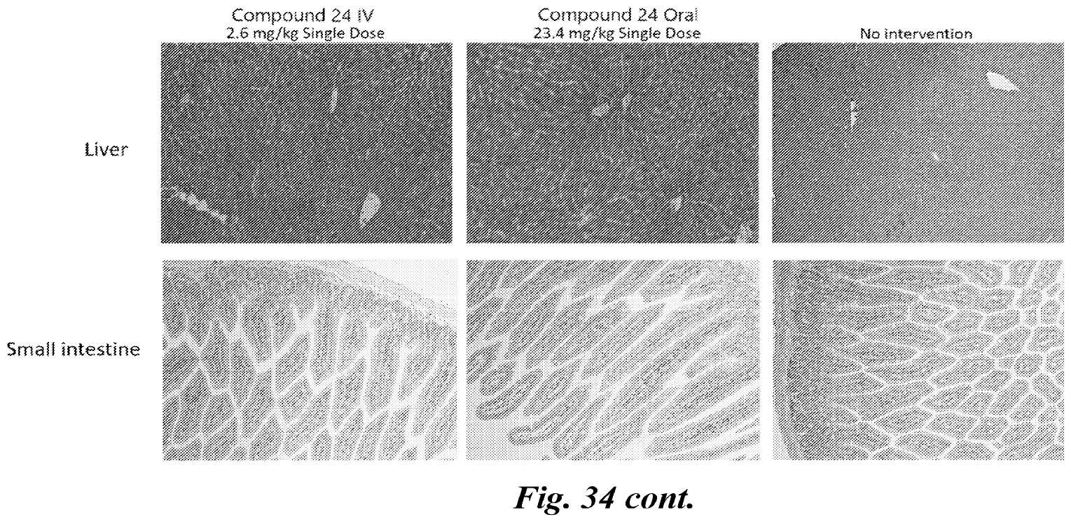

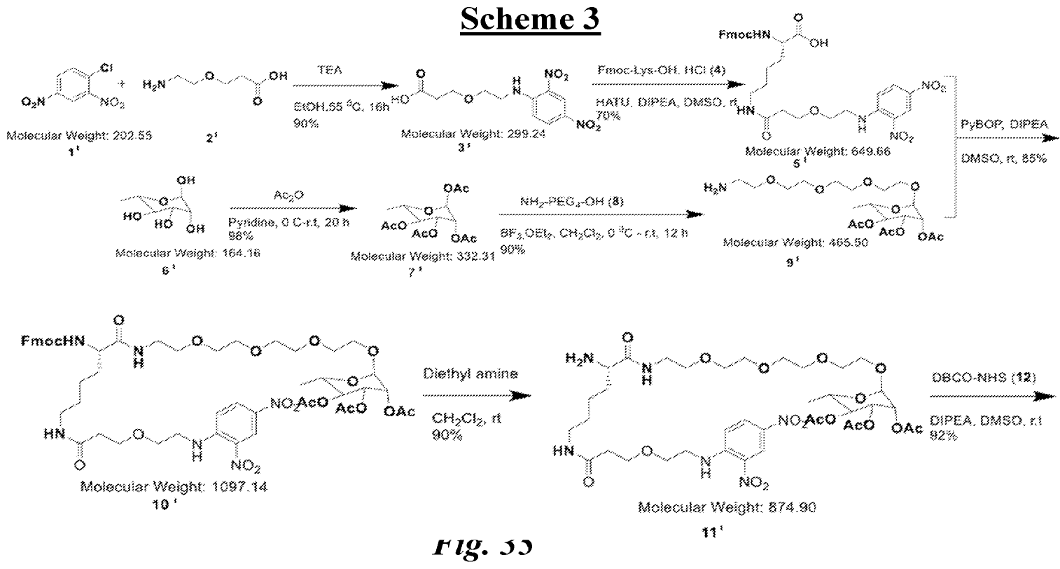

Serum cytokine and chemokine levels were also measured to assess the presence or absence of systemic inflammation in the subjects. FIG. 32 shows graphs of specific cytokine and chemokine expression levels in serum measured for each group at both the 48 hpi and 96 hpi treatment groups, where A is PBS control, B is Compound 24 treatment group via single dose IV, and C is Compound 24 treatment group via single dose OG.

Compound 24 treatment groups all exhibited lower levels of inflammatory cytokines as compared with the other treatment groups and control.

### Example 19

### Synthesis of a Folate-Dual-Hapten Conjugate (Compound 150)

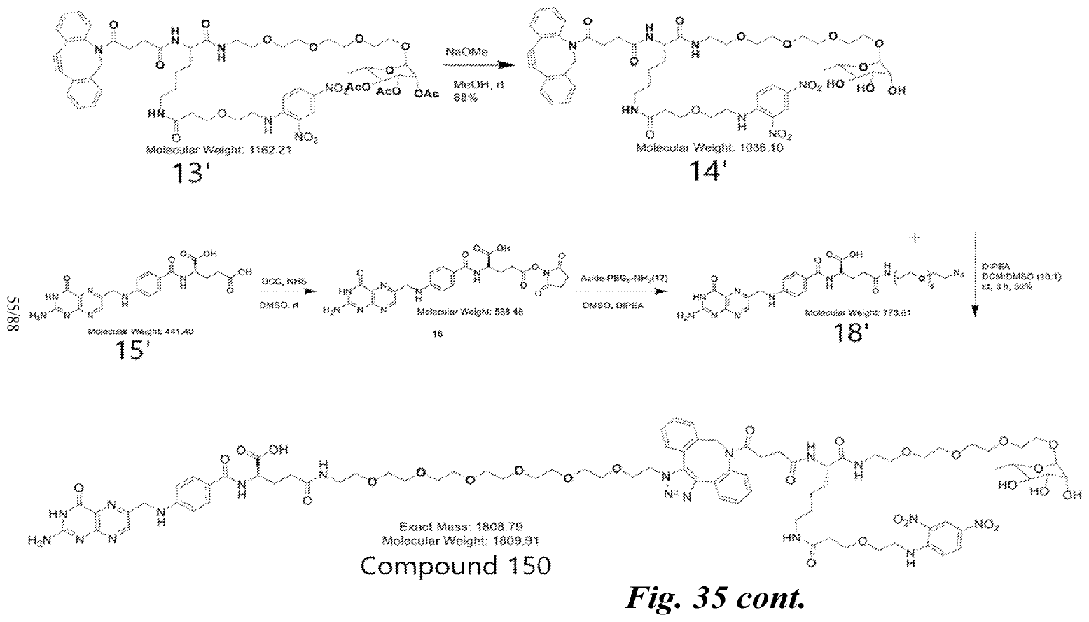

FIG. 35 shows the synthetic scheme for the synthesis of Compound 150.

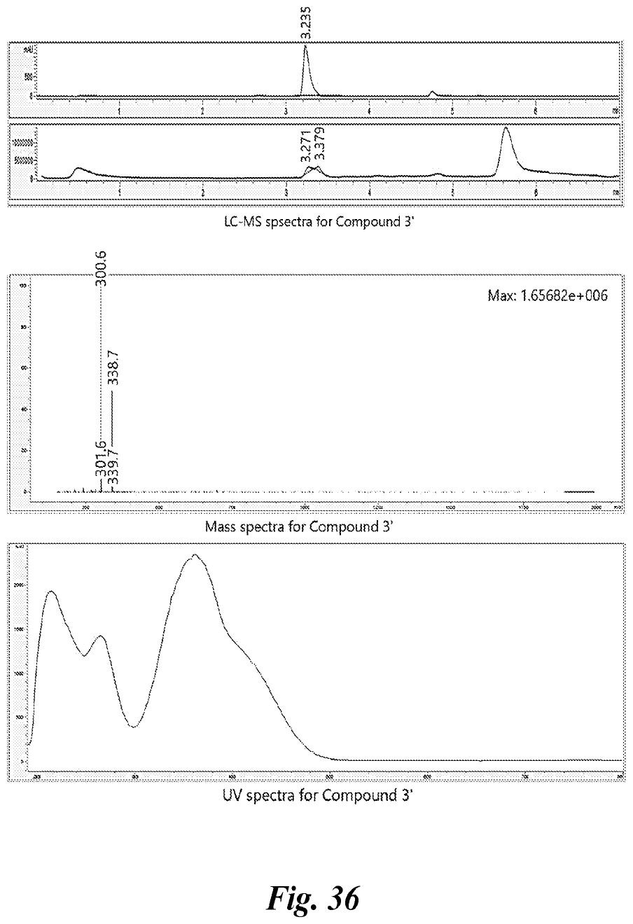

Synthesis of Compound 3′: Compounds 1′ and 2′ were prepared in accordance with the procedures set forth in Ref Chemistry—An Asian Journal 7(2): 272-276 (Theresa Kueckmann et al. eds.) (2012). Thereafter, TEA (1.38 mL, 9.87 mmol) was added to a solution of 1-chloro-2,4-dinitrobenzene (Compound 1′) (0.5 g, 2.47 mmol) and 3-(2-aminoethoxy) propanoic acid (Compound 2′, 0.33 g, 2.47 mmol) dissolved in EtOH (25 mL). The reaction mixture was heated to 55° C. for 16 hours and reaction progress was monitored by LC-MS. After completion of reaction was confirmed by the disappearance of one of the starting materials, i.e. dinitrobenzene, the reaction mixture was cooled and concentrated under reduced pressure. The crude mixture was purified by flash column chromatography on a Teledyne CombiFlash Rf+Lumen (silica-gel, 12 g column, 0-20% methanol in DCM) to yield Compound 3′ as a yellow solid, yield, 90%). LC-MS [M+H]+=300.24 (see FIG. 36).

Synthesis of Compound 5′: To a solution of 3-(2-((2,4-dinitrophenyl)amino)ethoxy)propanoic acid (Compound 3′, 0.1 g, 0.33 mmol) in dimethyl sulfoxide (2 mL) was added HATU (0.11 g, 0.28 mmol, 0.85 equiv.) followed by DIPEA (0.29 mL, 1.67 mmol, 5.0 equiv.) under argon atmosphere and stirred for 10 minutes at room temperature. Fmoc-Lys-OH. HCl (procured from Chem-Impex International, Wood Dale, IL; Compound 4′, 0.11 g, 0.27 mmol, 0.8 equiv.) was added to the reaction mixture and stirred for 2-3 hours at room temperature, and progress of the reaction was monitored by LC-MS.

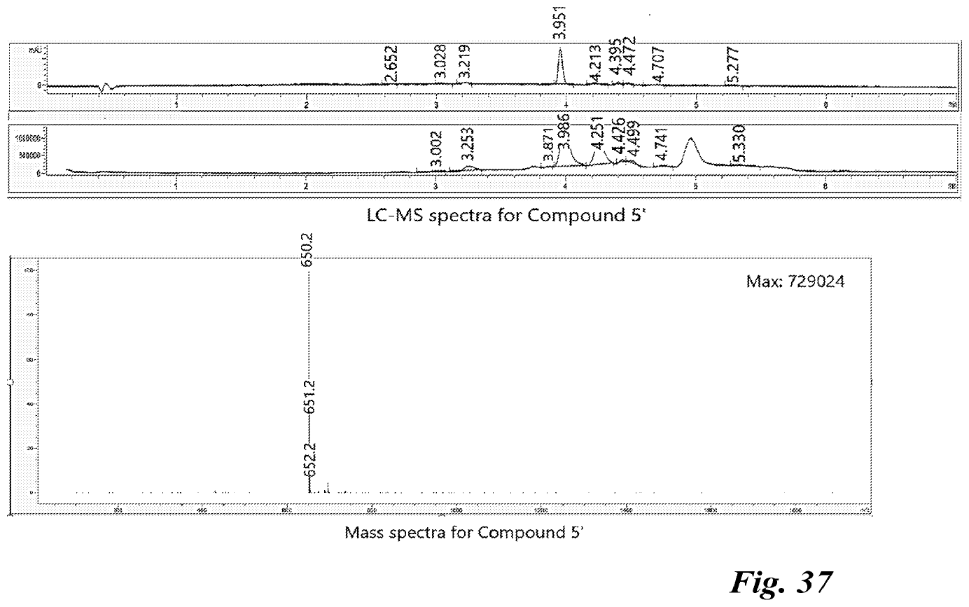

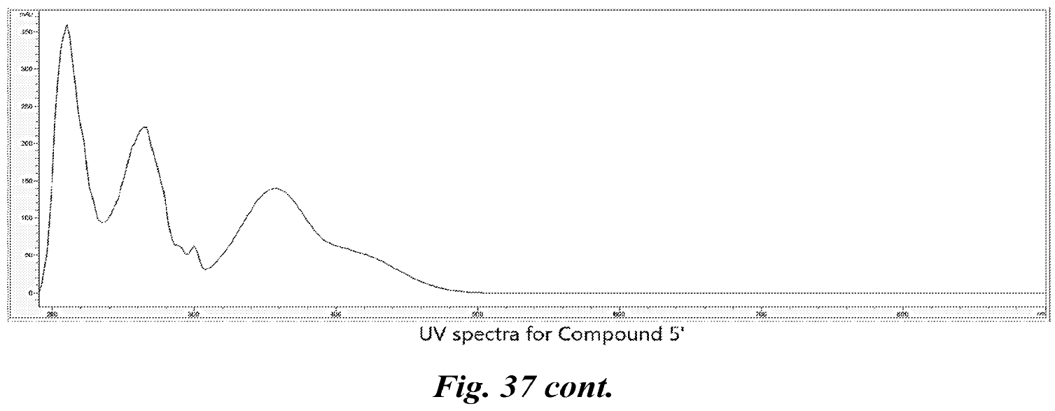

After completion of the reaction was confirmed by LC-MS, the reaction mixture was quenched by adding water and extracting with ethyl acetate. The combined organic layers were washed with brine, dried over anhydrous sodium sulfate, and concentrated under vacuum. The resulting crude was purified by silica-gel (4 g) column chromatography on Teledyne CombiFlash Rf+Lumen (0-10% methanol in DCM) and fractions were analyzed by LC-MS. The solvent was evaporated from the combined pure fractions using rotaevaporator under reduced vacuum and Compound 5′ was isolated in 70% yield. LC-MS [M+H]+=650.67 (see FIG. 37).

Synthesis of, 2,3,4-Tetra-O-Acetyl-α-L-Rhamnose (Compound 7′): Compound 7′ was prepared pursuant to the procedures set forth in De Coen et al., Synthetic rhamnose glycopolymer cell-surface receptor for endogenous antibody recruitment, Biomacromolecules 21(2): 793-802 (2020)). Briefly, α-L-Rhamnose monohydrate (6, 1.0 g, 5.49 mmol) was dissolved in 9.2 mL of anhydrous pyridine. The solution was stirred in an ice bath and purged with nitrogen prior to the dropwise addition of acetic anhydride (4.15 mL, 43.92 mmol, 8.0 equiv.) for 15 minutes by maintaining the internal temperature below 10° C. The reaction was allowed to warm to room temperature slowly in 2 hours and the progress of the reaction was monitored via TLC (Hexane/EtOAc, 65:35) and LC-MS, which indicated complete consumption of α-L-rhamnose monohydrate after 20 hours of reaction under an inert atmosphere. The reaction mixture was poured into ethyl acetate and extracted twice with 1.0 M HCL. The combined organic layers were washed with saturated sodium carbonate solution, water and brine, dried over anhydrous sodium sulfate, and concentrated under vacuum. The resulting crude oil product, Compound 7′ (yield, 98%) was used in the next step. LC-MS [M+H]+=333.32 and/or LC-MS [M+H2O]=350.32.

Synthesis of Compound 9′: Compound 9′ was prepared pursuant to the procedures set forth in De Coen et al. (2020), supra. Briefly. 1,2,3,4-Tetra-O-acetyl-α-L-rhamnose (7, 0.50 g, 1.50 mmol) was dissolved in DCM (7.5 mL) prior to the addition of —H₂N-PEG₄-OH (procured from BroadPharm, San Diego, CA; 8, 0.35 g, 1.81 mmol, 1.2 equiv.) under an inert atmosphere. The reaction flask was placed in an ice bath, and boron trifluoride diethyletherate (0.56 mL, 4.51 mmol, 3.0 equiv.) was added dropwise over 30 minutes at 4° C. The reaction mixture was stirred at ice bath temperature for 2 hours before the reaction was allowed to warm to room temperature. The progress of the reaction was monitored by TLC (Hexane/EtOAc, 30:70, Rf=0.30) and LC-MS, which indicated complete consumption of 1,2,3,4-tetra-O-acetyl-α-L-rhamnose (Compound 7′) after 16 hours of reaction. The reaction mixture was poured into ice water and extracted with DCM (3×10 mL). The combined organic layers were washed twice with saturated sodium bicarbonate solution, water, and brine, dried over anhydrous sodium sulfate, and concentrated under reduced pressure. The resulting crude was purified by silica-gel (12 g) column chromatography on Teledyne CombiFlash Rf+Lumen (0-10% methanol in DCM) and product (Compound 9′) was isolated, yield, 90%. LC-MS [M+H]+=466.51.

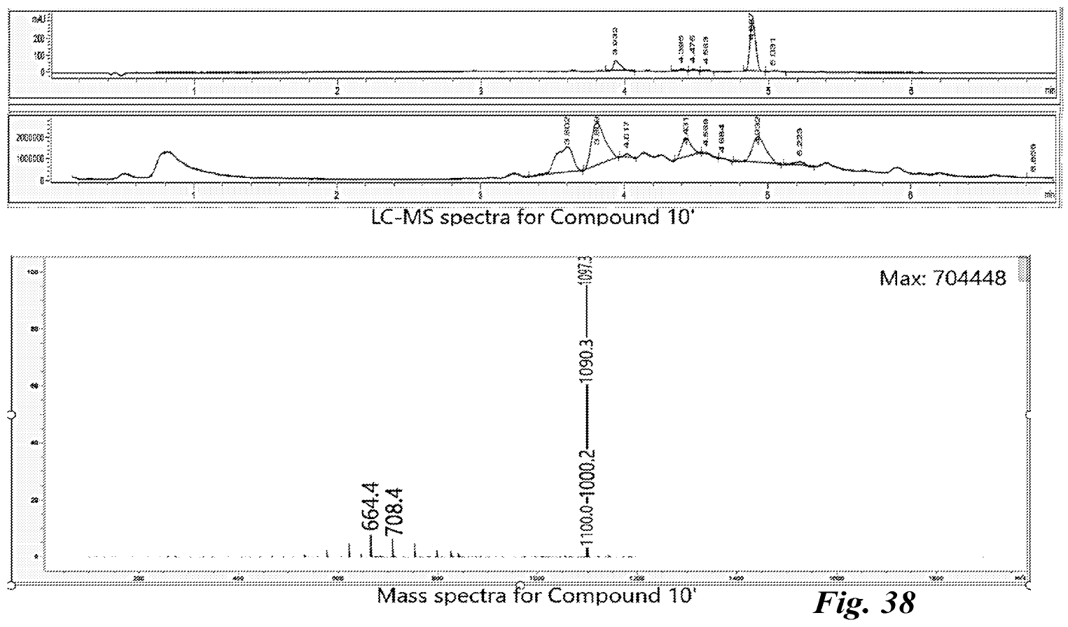

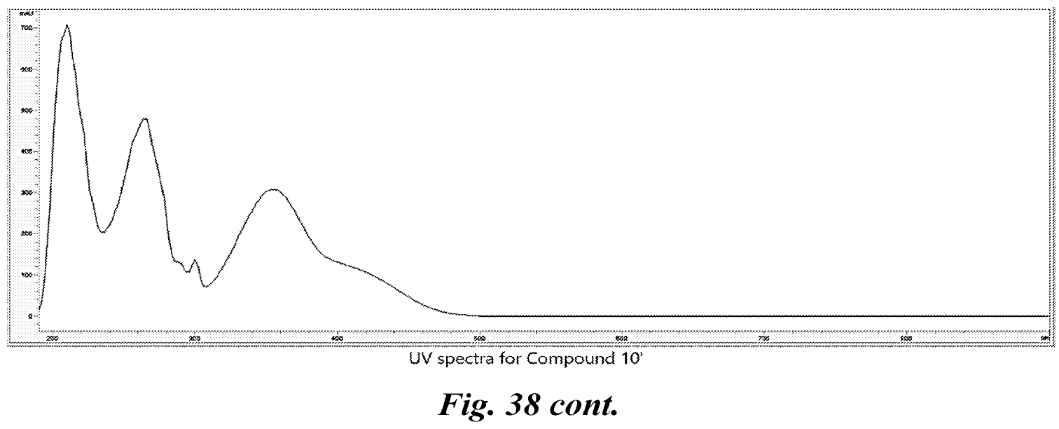

Synthesis of Compound 10′: To a solution of acid (Compound 5′, 0.06 g, 0.09 mmol) and Rhamnose (OAc)₃-PEG₄-NH₂ (Compound 9′, 0.04 g, 0.09 mmol, 1.0 equiv.) in dimethyl sulfoxide (1.5 mL) was added PyBOP (0.05 g, 0.10 mmol, 1.1 equiv.) followed by DIPEA (0.081 mL, 0.46 mmol, 5.0 equiv.) under argon atmosphere at room temperature. Progress of the reaction monitored by LC/MS. After completion of the reaction was confirmed by LC-MS, the reaction mixture was quenched by adding water and extracting with ethyl acetate. The combined organic layers were washed with brine, dried over anhydrous sodium sulfate, and concentrated under vacuum. The resulting crude was purified by silica gel (4 g) column chromatography on Teledyne CombiFlash Rf+Lumen (0-10% MeOH in dichloromethane) and product (Compound 10′) was isolated in 93% yield. LC-MS [M+H]+=1098.15 (see FIG. 38).

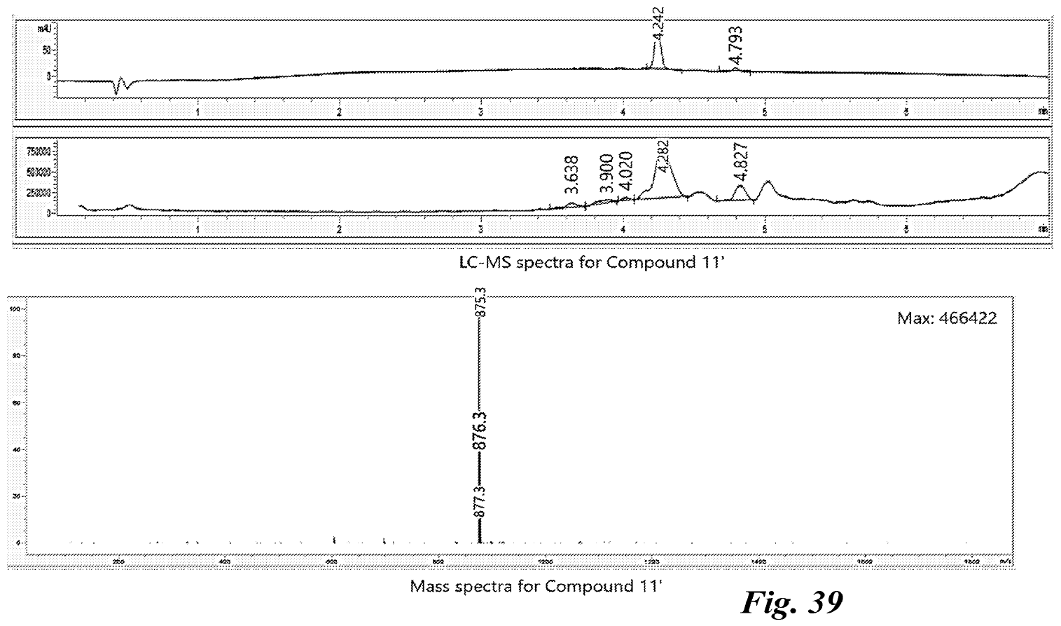

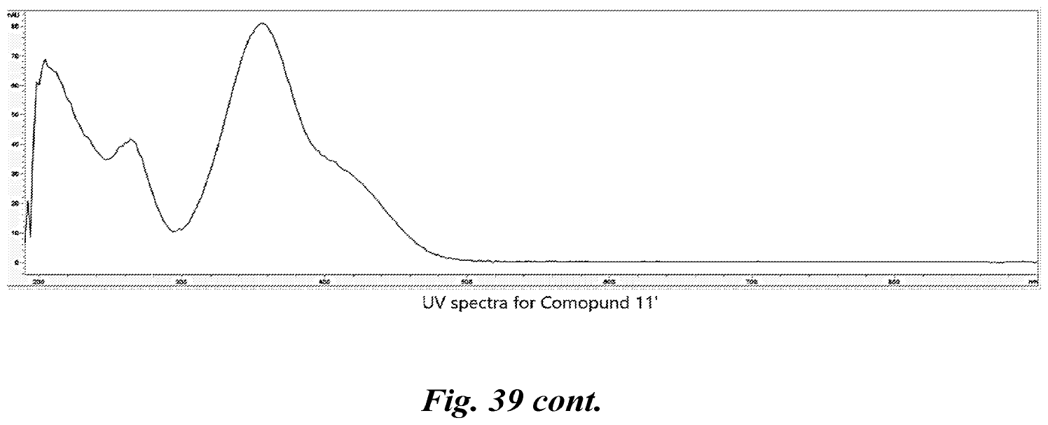

Deprotection of -Fmoc group to Synthesize Compound 11′: To Compound 10′ (0.01 g, 0.01 mmol) in dry DCM (0.2 ml) was added DEA (100 uL) under argon at room temperature. The solution stirred for 1 hour at room temperature until the reaction was complete as demonstrated by LC-MS. The DEA was removed by rotary evaporation under reduced pressure and the crude product precipitated in diethyl ether to give as a yellow solid with a quantitative yield for use with the next step without further purification. LC-MS [M+H]+=875.91 (see FIG. 39).

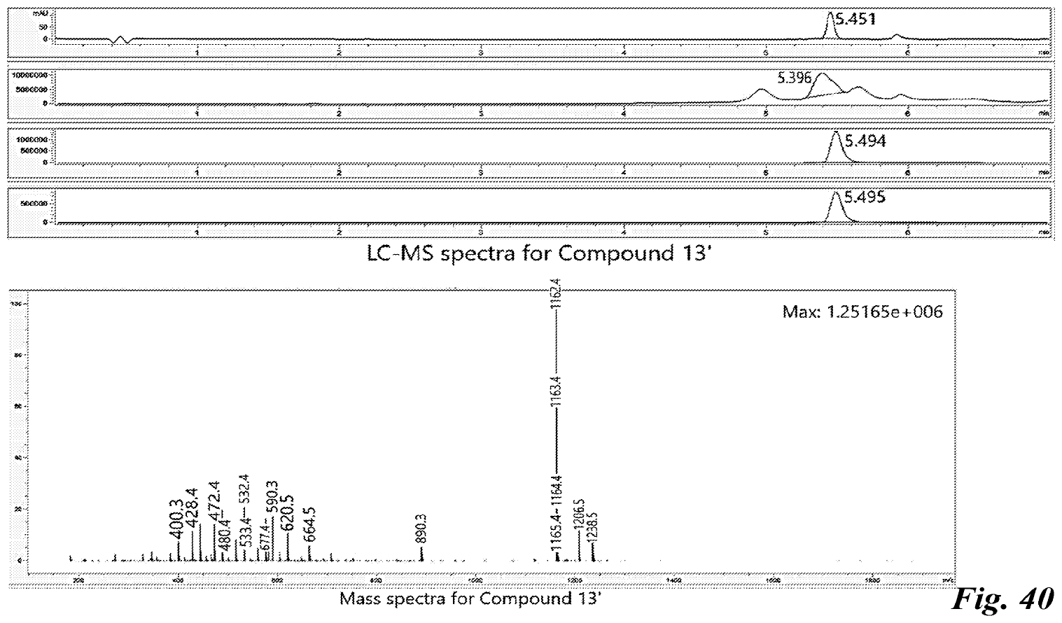

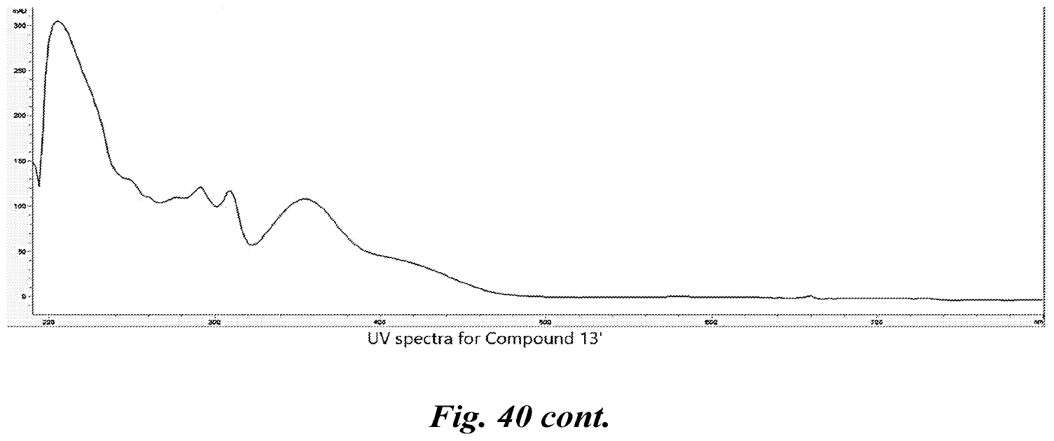

Synthesis of Compound 13′: To a solution of DBCO-NHS (procured from BroadPharm, San Diego, CA; Compound 12′, 0.012 g, 0.029 mmol, 1.05 equiv.) and Compound 11 (0.024 g, 0.027 mmol, 1.0 equiv.) dissolved in DCM:DMSO (10:1, 0.6 mL) was added DIPEA (0.048 l, 0.27 mmol, 10 equiv.) dropwise under argon. The reaction mixture stirred under room temperature for 2-3 hours and progress of the reaction was monitored by LC-MS. After completion of the reaction, which was confirmed by complete consumption of Compound 11′, the solvent was evaporated by rotary evaporator under reduced pressure and the crude was purified by silica-gel (4 g) column chromatography on Teledyne CombiFlash Rf+Lumen (0-10% methanol in DCM) to afford Compound 13′ as a yellowish solid, yield, 93%). LC-MS [M+H]+=1163.21 (see FIG. 40).

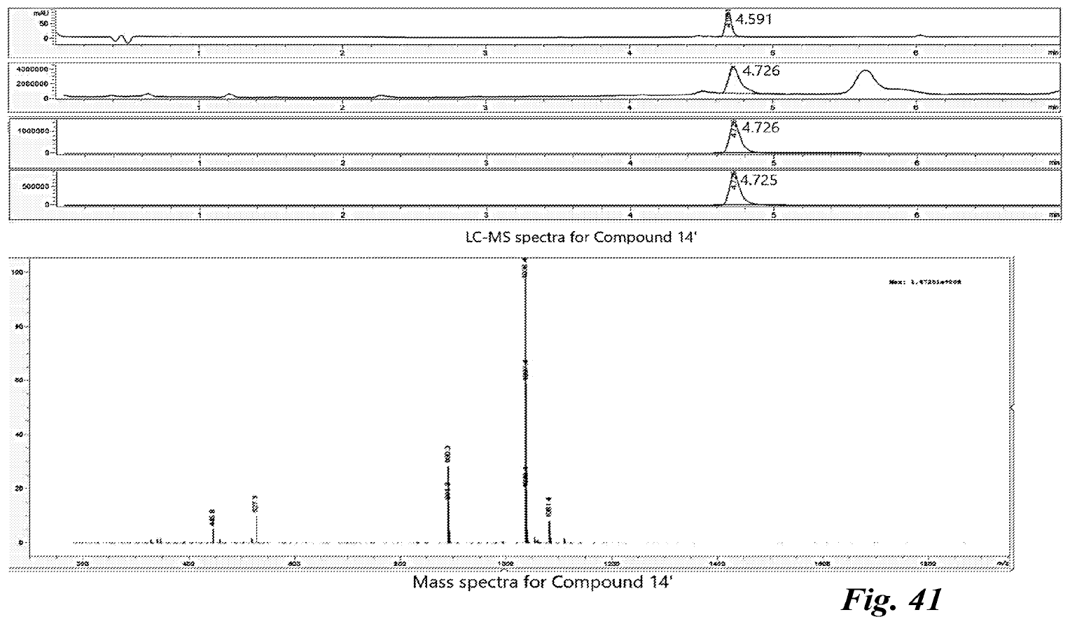

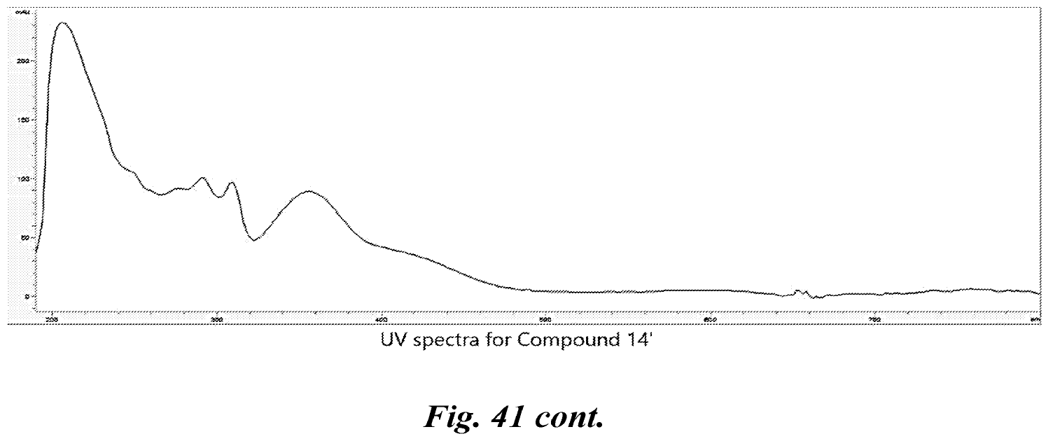

Synthesis of Intermediate 14′: Compound 13′ (0.025 g, 0.022 mmol) was dissolved in anhydrous MeOH (1 mL) and treated drop wise with 0.5 M NaOH (25 μL). The reaction mixture was stirred at room temperature for 1 hour at which time LC-MS analysis revealed the absence of starting material. The reaction was neutralized by adding Dowex® 50WX8 (H+) resin, filtering, and concentrating under reduced pressure. The yellowish crude product (Compound 14′) used directly for the next step without further purification. LC-MS [M+H]+=1037.11 (see FIG. 41).

Synthesis of Folate-NHS ester (Compound 16): DCC (0.281 g, 1.359 mmol, 1.2 equiv.) at r.t under argon to a solution of Compound 15′ (0.50 g, 1.133 mmol) and N-hydroxysuccinimide (0.143 g, 1.246 mmol, 1.10 equiv.) in DMSO (12 mL), and the solution was stirred for 12 hours. Progress of the reaction was monitored by LC/MS. After completion of the reaction was confirmed by LC-MS, the reaction mixture was precipitated in acetone (15×) and the product was isolated by centrifugation. Further, the crude product was washed with acetone (2×50 mL) and EtOAc (1×30 mL), dried under vacuum, and the crude product (Compound 16) was used for the next step without further purification.

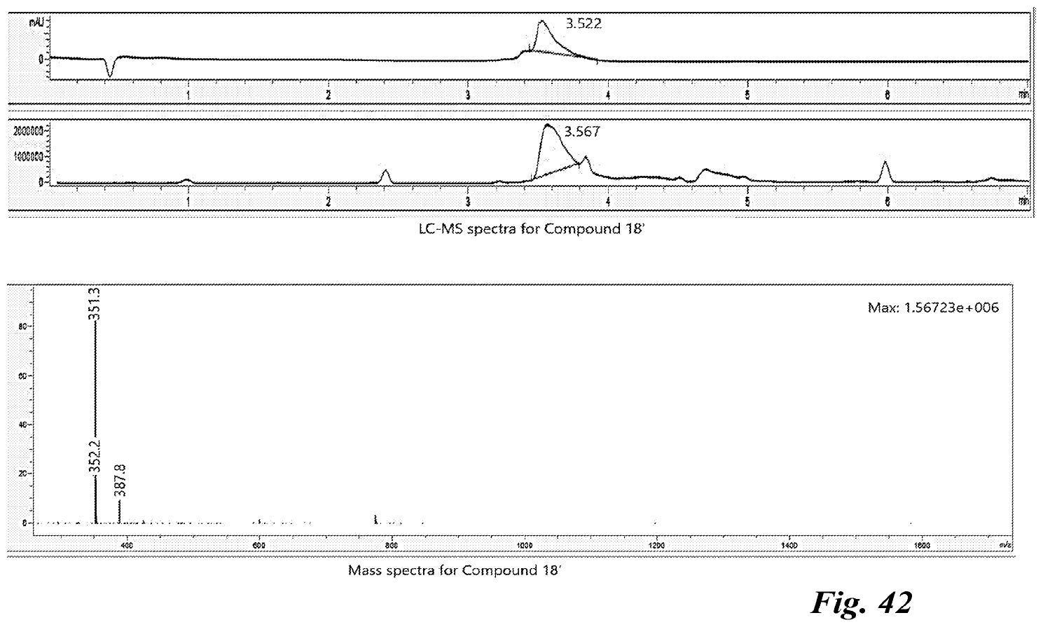

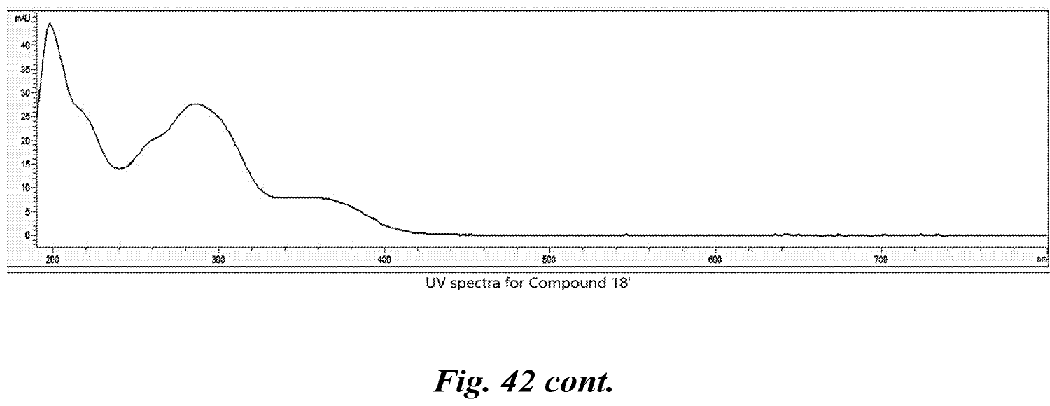

Synthesis of Folate-PEG₆-azide (Compound 18′): To a solution of Folate-NHS ester (Compound 16, 0.05 g, 0.093 mmol) in dry DMSO (1.0 mL) was added N₃-PEG₆-NH₂, (Compound 17′ procured from BroadPharm, San Diego, CA; 0.032 g, 0.093 mmol, 1.05 equiv.), followed by DIPEA (0.081 mL, 0.46 mmol, 5.0 equiv.) at room temperature under argon and stirred for 12 hours. Progress of the reaction was monitored by LC/MS. After completion of the reaction was confirmed by LC/MS, the reaction mixture was precipitated by adding diethyl ether and centrifuged. The yellow precipitate was collected and washed with diethyl ether (2×15 mL) and crude mass purified by prep-HPLC on a C18 column (5-95% B over 60 minutes, flow 7 mL/min; B: acetonitrile; A: 20 mM NH₄OAc, pH 7 buffer, UV @, 280 nm) to afford Compound 18′. LC-MS [M+H]+=774.83 (see FIG. 42).

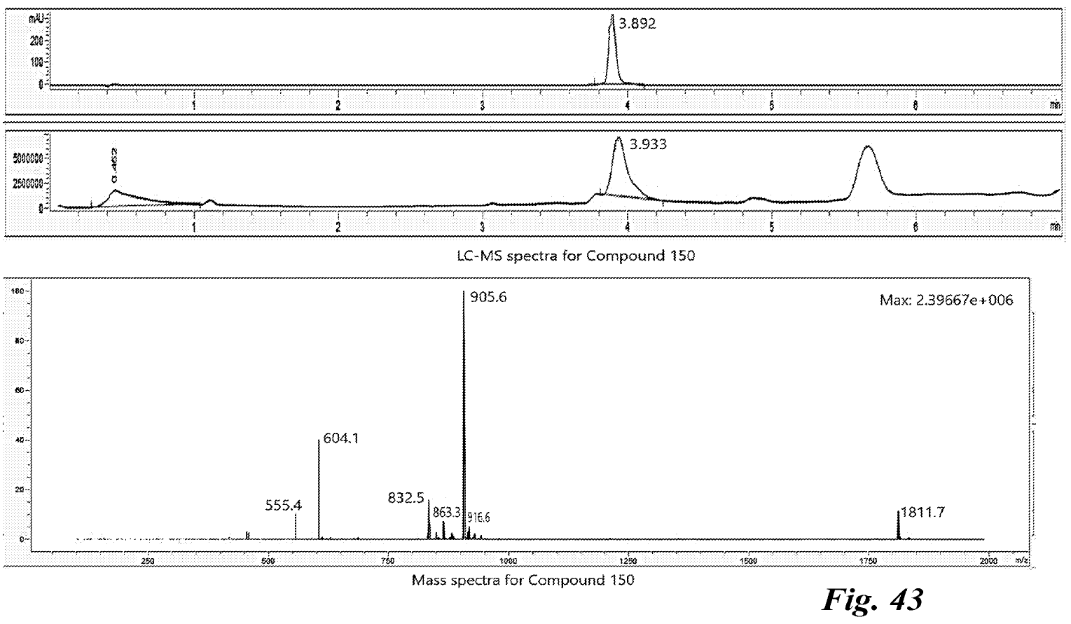

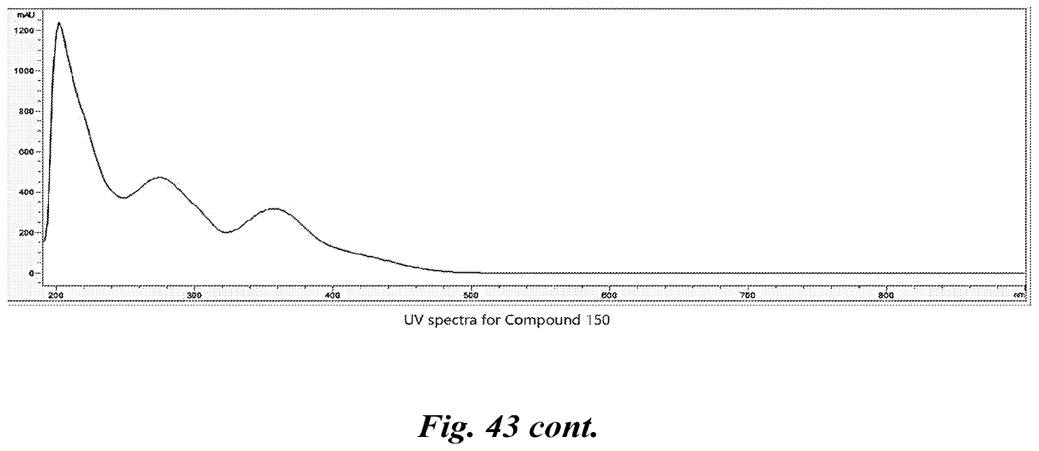

Synthesis of Compound 150: To a solution of Compound 18′ (10 mg, 0.013 mmol) and Compound 14′ (13.4 mg, 0.013 mmol, 1.0 equiv.) in dry DMSO (1.0 mL) was added DIPEA (12 l, 0.065 mmol, 5.0 equiv.) dropwise under argon. The reaction mixture was stirred at room temperature for 2-4 hours and the progress of the reaction was monitored by LC-MS. After completion of the reaction, the crude product was purified by prep-HPLC on a C18 column (5-95% B over 60 minutes, flow 7 m/min; B: acetonitrile; A: 20 mM NH4OAc, pH 7 buffer, UV @360 nm and 280 nm) to afford final compound, Compound 150 and yield, 24%. Purity: 97% by HPLC and LC-MS [M+H]+=1810.93 (see FIG. 43).

### Example 20

### In Vitro Functional Assays with Compound 150

Folate receptor expressing murine lung cancer cells (cell line M109) were seeded on a 96-well plate at a density of 500 cells/well and allowed to adhere overnight at 37° C. The cells were then incubated with serial dilutions of the folate-dual-hapten conjugate Compound 150 for 2 hours at 37° C., following which human serum was added to assess the effect on complement dependent cytotoxicity (CDC), if any.

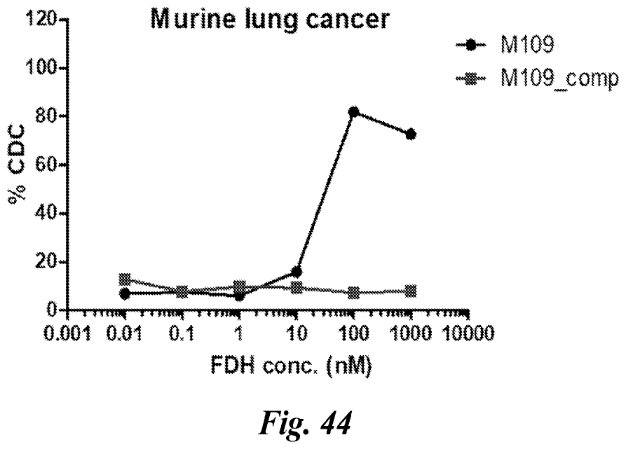

The results are shown in FIG. 44, which support that the addition of Compound 150 induced CDC, likely by recruiting anti-hapten antibodies on the cancer cell surface followed by activation by the antibodies of the complement system in the human serum. 100-fold excess of folate-glucosamine was used as competitor in the control set to confirm that the observed immunogenic effect was indeed due to the binding of folate-receptor on the target cells and folate moiety on the dual hapten conjugate (identified as M109_comp in FIG. 44).

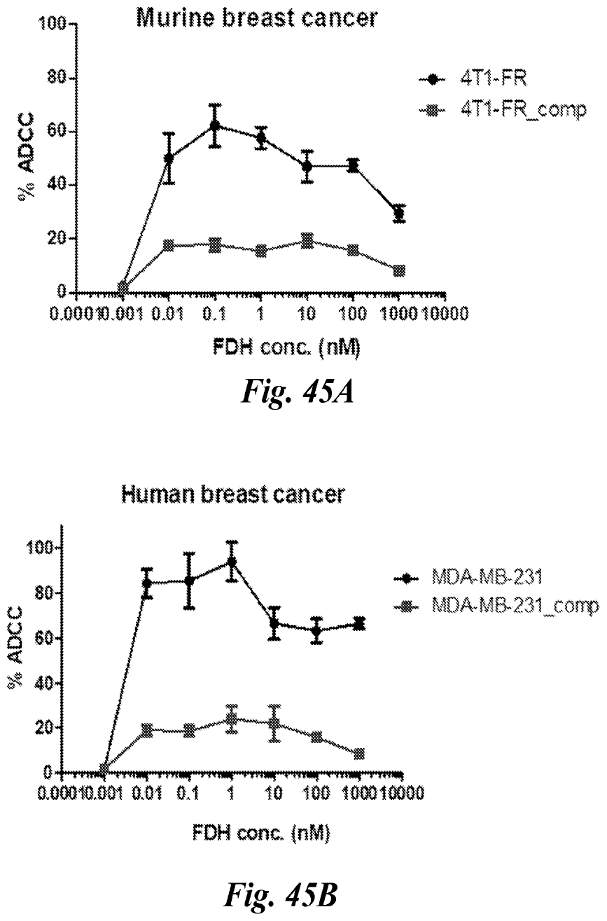

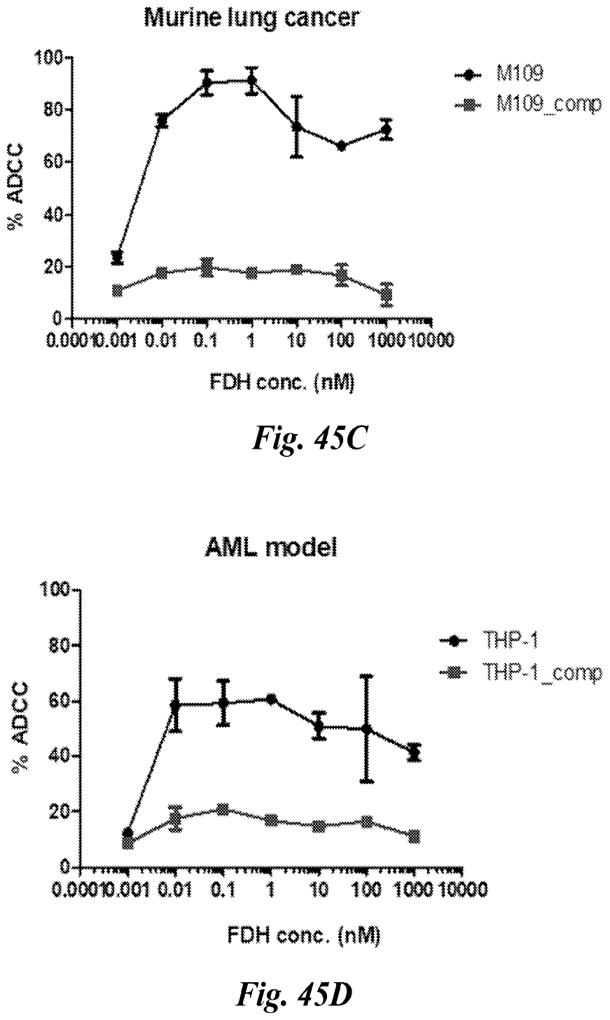

Additionally, folate-receptor expressing cancer cells (4T1 (murine breast cancer cells), MDA-MB-231 (human breast cancer cells), M109 (murine lung cancer cells)), and THO-1 (AML model) were assayed for ADCC activity of Compound 150. After a 2 hour incubation with serial dilutions of Compound 150, the cells were then incubated with IVIg for 2 hours at 37° C., and human Fc-γ-RIII expressing effector cells for overnight at 37° C., 5% CO₂. The results are shown in FIGS. 45A-45D, which support that Compound 150 induced ADCC by recruiting anti-hapten antibodies on the cancer cell surface, followed by activation of the Fc-γ-RIII receptors expressed on the effector cells.

### Example 21

### Efficacy In Vivo Studies of Compound 150 in Murine Lung Cancer Model

6-8-week-old female Balb/c mice were inoculated with 2×10⁶ cells/mouse of M109 murine lung cancer cell line (folate receptor expressing murine lung cancer cells) subcutaneously in the right flank. After 10 days of inoculation, when tumor size reached ˜50 mm³, treatment was started with the following test articles: (a) 100 μL/mouse PBS only; (b) a SOC treatment (i.e., 200 μg/mouse 3 days/week anti-PD1+chemo (50 mg/kg intraperitoneal injection of carboplatin on day 18 post tumor inoculation+36 mg/kg intravenous injection of paclitaxel on day 18-22 post tumor inoculation)); or (c) 10 nmol/mouse Compound 150, each a daily dose for 5 days/week. The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study.

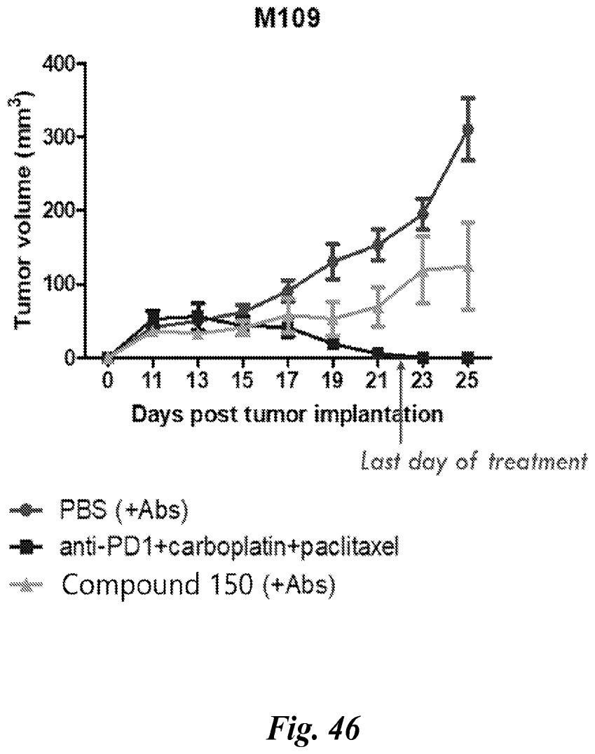

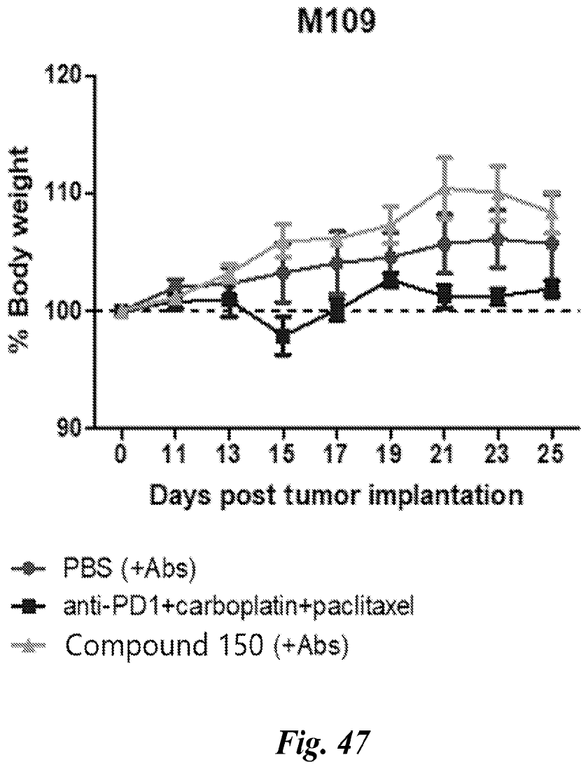

Tumors were measured every other day and mice were sacrificed 25 days post tumor implantation. FIGS. 46 and 47 show tumor volume (mm³) versus days post tumor implantation and body weight percentage of the subjects versus days post tumor implantation, respectively. The data supports Compound 150 suppressed tumor growth in the M109 model and shows promise as a monotherapy in lung cancer treatment.

### Example 22

### Efficacy In Vivo Studies of Compound 150 in Human Lung Cancer (Cold Tumor) Model

6-8-week-old female C57BL/6 mice were inoculated with 5×10⁶ cells/mouse of LLC-1 human lung cancer cell line (LLC-1) subcutaneously in the right flank. After 10 days of inoculation, when tumor size reached ˜50 mm³, treatment was started with the following test articles: (a) 100 L/mouse PBS only; (b) a SOC treatment (i.e., 200 μg/mouse intraperitoneal injection of anti-PDL1 3-days/week+2 Gy/mouse 3 days/week radiotherapy); or (c) 10 nmol/mouse Compound 150, each a daily dose for 5 days/week. The PBS and Compound 150 groups also were given an intraperitoneal injection ofanti-DNP and anti-Rhanmose antibodies on days 10, 14, 17 and 21 post-implantation to achieve humanized titer of anti-hapten antibodies over the two-week period.

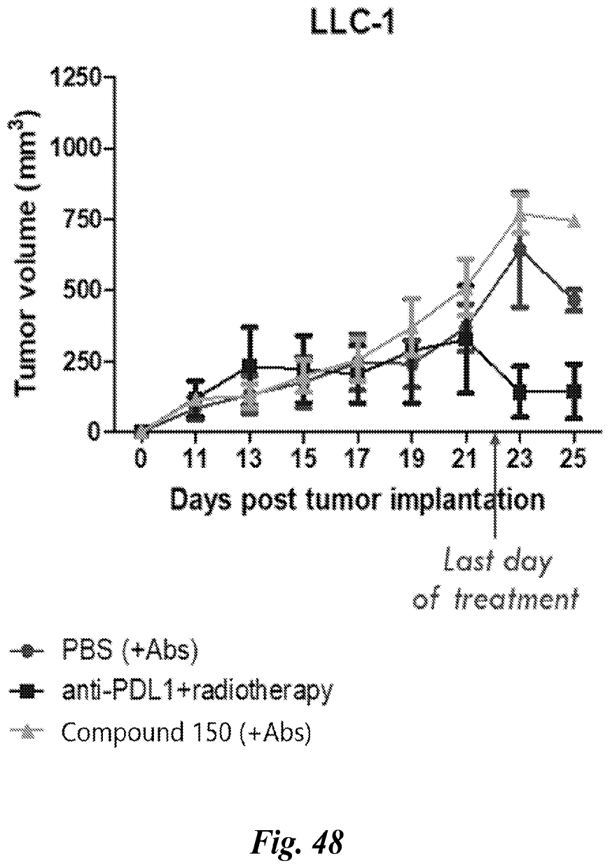

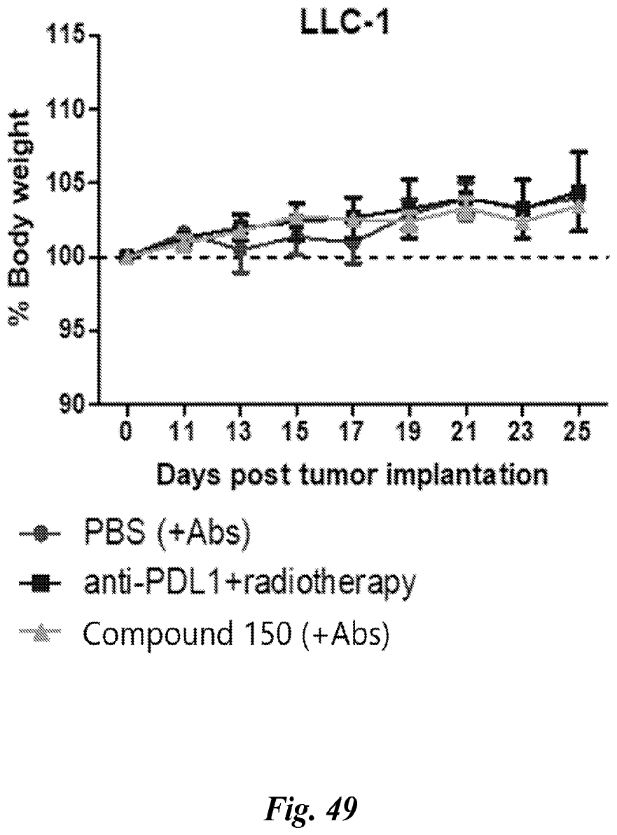

Tumors were measured every other day and mice were sacrificed 25 days post tumor implantation. FIGS. 48 and 49 show tumor volume (mm³) versus days post tumor implantation and body weight percentage of the subjects versus days post tumor implantation, respectively. The data supports Compound 150 may benefit from combination with therapies that turn cold tumors into hot tumors.

### Example 23

### Efficacy In Vivo Studies of Compound 150 in Murine Colorectal Cancer Model

6-8-week-old female Balb/c mice were implanted with 2×10⁶ cells/mouse of CT26 murine colorectal carcinoma cell line subcutaneously in the right flank. After 10 days of inoculation, when tumor size reached ˜50 mm³, treatment was started with the following test articles: (a) 100 μL/mouse PBS only; (b) a SOC treatment (i.e., Leucovorin 100 mg/kg intraperitoneal injection once a week+5-FU 50 mg/kg once a week+oxaliplatin 6 mg/kg on day 8 and day 10 post tumor inoculation); or (c) 10 nmol/mouse Compound 150, each a daily dose for 5 days/week. The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study.

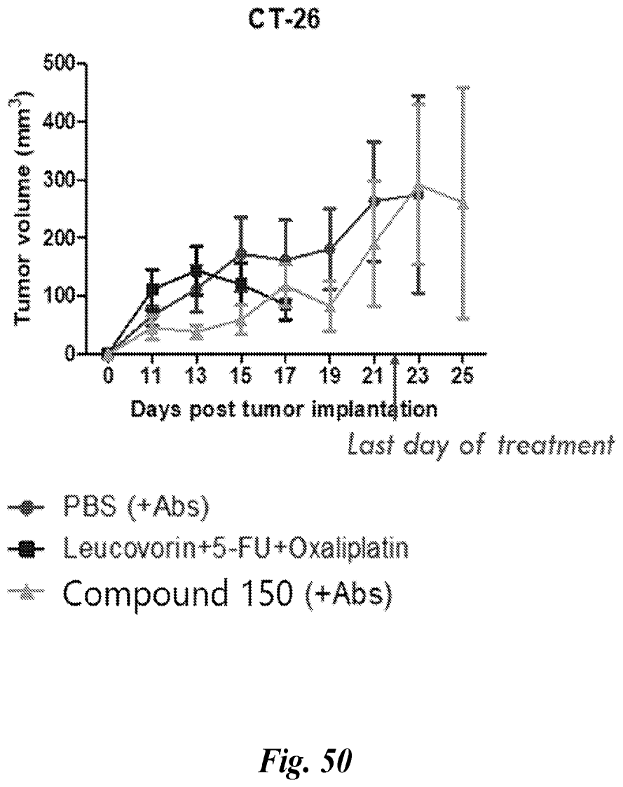

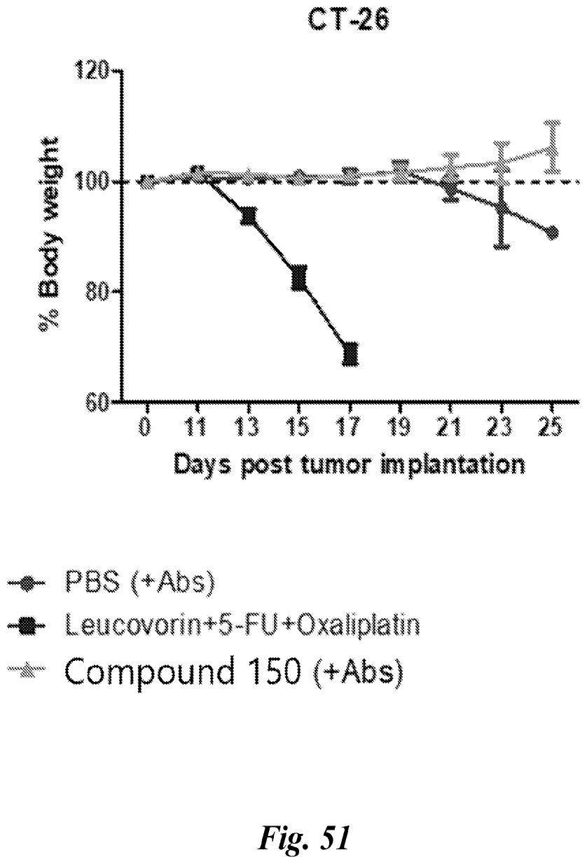

Tumors were measured every other day and mice were sacrificed 25 days post tumor implantation. FIGS. 50 and 51 show tumor volume (mm³) versus days post tumor implantation and body weight percentage of the subjects versus days post tumor implantation, respectively. The mice that showered greater than 25% body weight loss were euthanized as humane end points.

All mice died by day 17 in the Leucovorin+5-FU+Oxaliplatin group, but in the Compound 150 cohort, all mice survived with a slower rate of tumor growth as compared to the untreated control.

### Example 24

### Evaluation of Multiple Combination Therapies in Murine Lung Cancer Model

6-8-week-old female Balb/c mice (n=5 per cohort) were implanted with 2-5×10⁶ cells of M109 murine lung cancer cell line (folate receptor expressing murine lung cancer cells) subcutaneously (day 0). At day 11 post implantation, treatment was started with the following test articles: (a) 10 μL/mouse PBS only; (b) chemo (carboplatin+paclitaxel)); (c) 10 nmol/mouse Compound 150+anti-PD1; (d) 10 nmol/mouse Compound 150 (+Abs); and (e) 10 nmol/mouse Compound 150+chemo. The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study. Tumors were measured every other day.

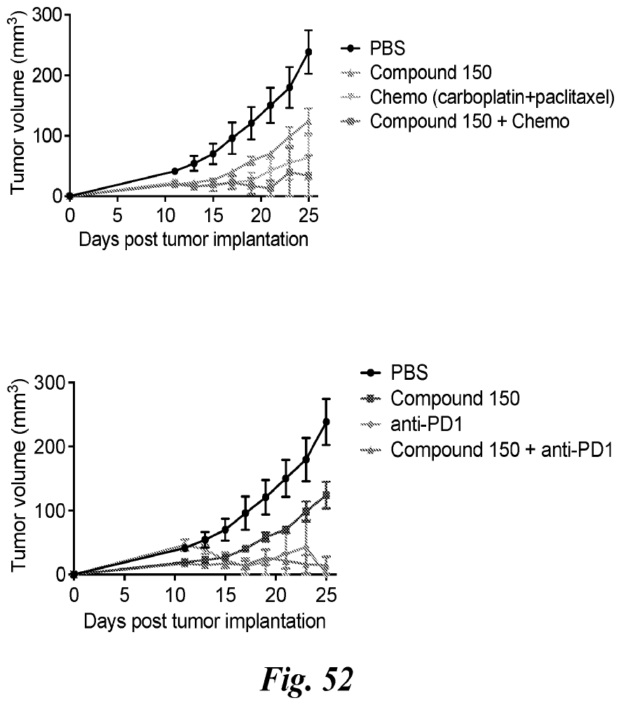

FIG. 52 shows tumor volume (mm³) versus days post tumor implantation. Two of five of the animals in the chemotherapy treatment cohort lost the tumors by day 17 post implantation (all animals in this cohort survived until the end of the study). Three of the five animals in the combination therapy Compound 150+anti-PD1 cohort lost the tumors (1 of the remaining animals did not survive day 22 post implantation). Four of the five animals in the combination therapy (Compound+chemotherapy) cohort lost the tumors (3 of the 5 animals did not survive day 22 post implantation). The data supports combination of Compound 150 with anti-PD-1 or chemotherapy significantly enhanced treatment efficacy.

A second study was conducted using the same protocol described above with the following test articles: (i) 100 μL/mouse PBS only; (ii) SOC treatment (anti-PD1+chemotherapy (carboplatin+paclitaxel)); (iii) 10 nmol/mouse Compound 150+sunitinib; (iv) 10 nmol/mouse Compound 150 (+Abs); and (v) 10 nmol/mouse Compound 150+folate-toll-like receptor 7 agonist conjugate (FA-TLR7). The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study.

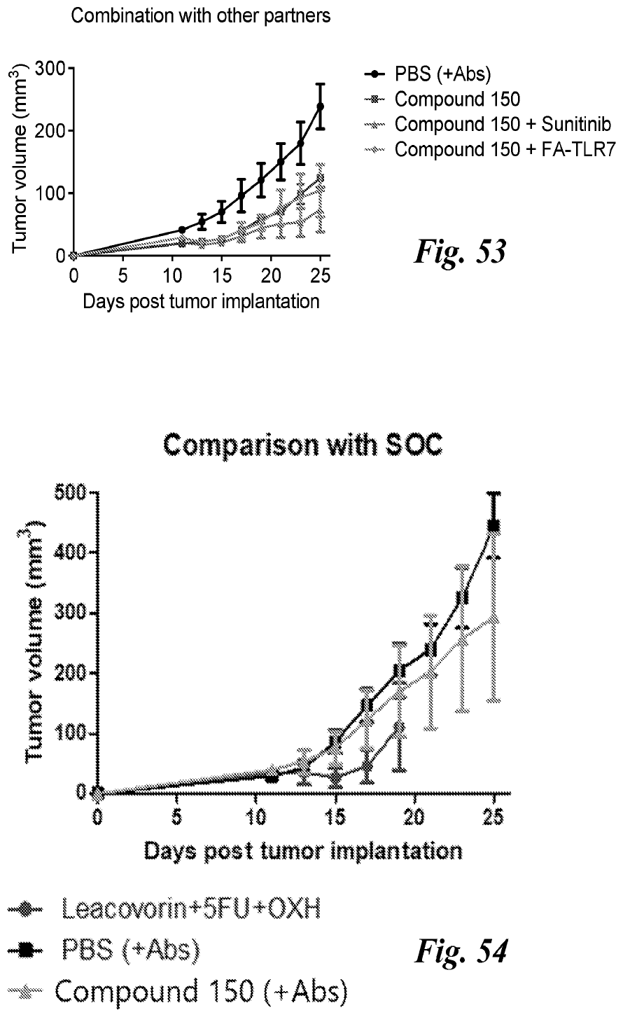

FIG. 53 shows tumor volume (mm³) versus days post tumor implantation. Four of the five of the animals in the SOC treatment cohort lost the tumors (all animals in this cohort survived until the end of the study). One of the five animals in the combination therapy Compound 150+sunitinib cohort was about to lose the tumor, but died on day 22 post implantation. One of the five animals in the combination therapy Compound+FA-TLR7 cohort lost the tumors (all animals in this cohort survived until the end of the study). The data support combination of Compound 150 with sunitinib slightly enhanced the anti-tumor efficacy of the treatment, but that combination of Compound 150 with FA-TLR7 at these doses did not achieve any statistical-relevant efficacy improvement.

### Example 25

### Comparison of Efficacy In Vivo Studies of Compound 150 in Murine Colorectal Cancer Model

6-8-week-old female Balb/c mice (n=5 per cohort) were implanted with 2×10⁶ cells/mouse of CT-26 murine colorectal cancer cell line subcutaneously (day 0). At day 11 post implantation, treatment was started with the following test articles: (a) PBS only; (b) Leacovorin+5FU+Oxaliplatin (OXH) (the SOC cohort); or (c) 10 nmol/mouse Compound 150, each a daily dose. Treatment ended on day 22 post implantation. The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study.

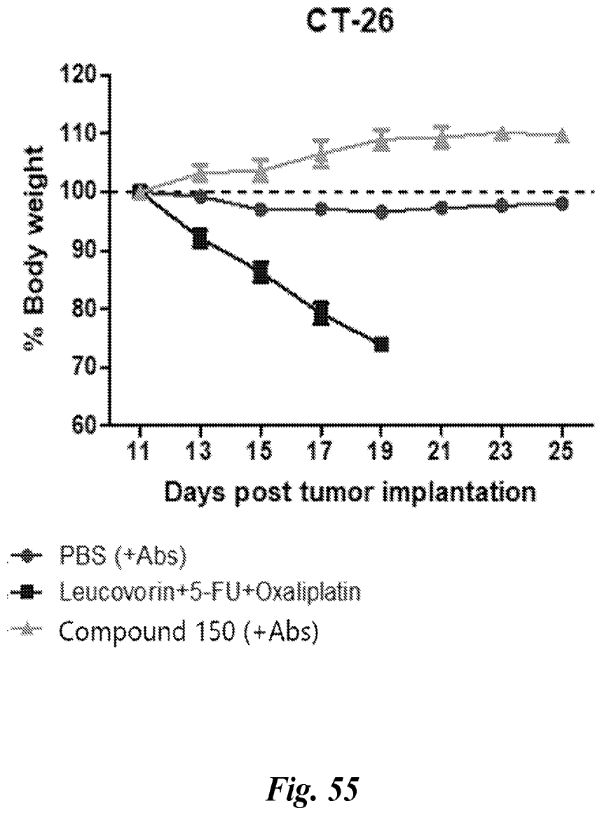

Tumors were measured every other day and mice were sacrificed 25 days post tumor implantation (all animals survived until the end of the study). FIGS. 54 and 55 show tumor volume (mm³) versus days post tumor implantation and body weight percentage of the subjects versus days post tumor implantation, respectively. Three of the five animals in the SOC cohort lost the tumors, and all animals reached a body weight of less than 75% by day 19 post implantation and were euthanized.

### Example 26

### Evaluation of Multiple Combination Therapies in Murine Colorectal Cancer Model

6-8-week-old female Balb/c mice (n=5 per cohort) were implanted with 2-5×10⁶ cells of CT-26 murine colorectal cancer cell line subcutaneously (day 0). At day 11 post implantation, treatment was started with the following test articles: (a) 100 μL/mouse PBS only; (b) chemo (carboplatin+paclitaxel)); (c) 10 nmol/mouse Compound 150+anti-PDT; (d) 10 nmol/mouse Compound 150 (+Abs); (e) 10 nmol/mouse Compound 150+chemo; and (f) SOC (Leacovorin+5FU+oxiplatin). The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study. Tumors were measured every other day.

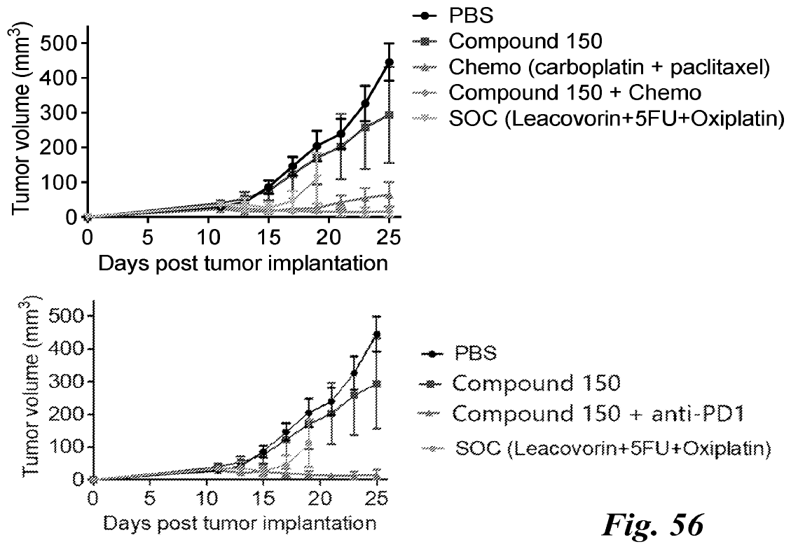

FIG. 56 shows tumor volume (mm³) versus days post tumor implantation. Four of the five animals in the combination therapy Compound 150+anti-PD1 cohort lost the tumors. Three of the five animals in the combination therapy Compound 150+chemotherapy cohort lost the tumors. Two of the five animals in the chemotherapy (alone) cohort lost the tumors. The data supports a combination treatment of Compound 150 with chemotherapy performs better than chemotherapy treatment alone, and a combination treatment of Compound 150 with anti-PD1 performs better than the SOC.

A second study was conducted using the same protocol described above with the following test articles: (i) 100 μL/mouse PBS only; (ii) Leacovorin+5FU+OXH (SOC); (iii) 10 nmol/mouse Compound 150+sunitinib; (iv) 10 nmol/mouse Compound 150 (+Abs); and (v) 10 nmol/mouse Compound 150+folate-TLR7 conjugate. The PBS and Compound 150 groups also were given an intraperitoneal injection of ani-DNP and anti-Rhammnose antibodies ondays 10, 14, 17 and 21 to achieve humanized titer of anti-hapten antibodies throughout the length of the study.

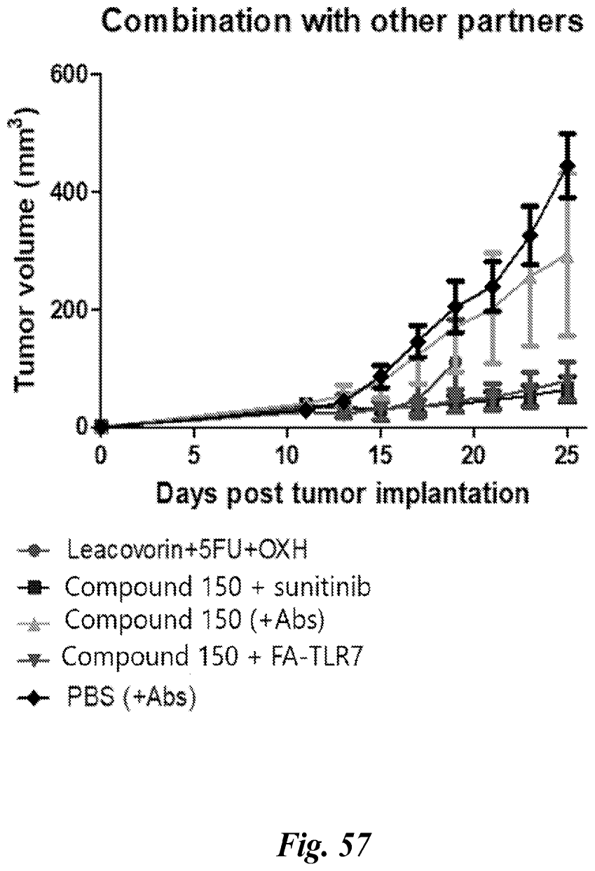

FIG. 57 shows tumor volume (mm³) versus days post tumor implantation. One of the five animals in the combination therapy treatment cohort Compound 150+sunitinib cohort lost the tumor. One of the five animals in the combination therapy Compound 150+FA-TLR7 cohort lost the tumor. The data supports combination therapy of Compound 150 with sunitinib or FA-TLR7 significantly enhanced the anti-tumor efficacy of the treatment.

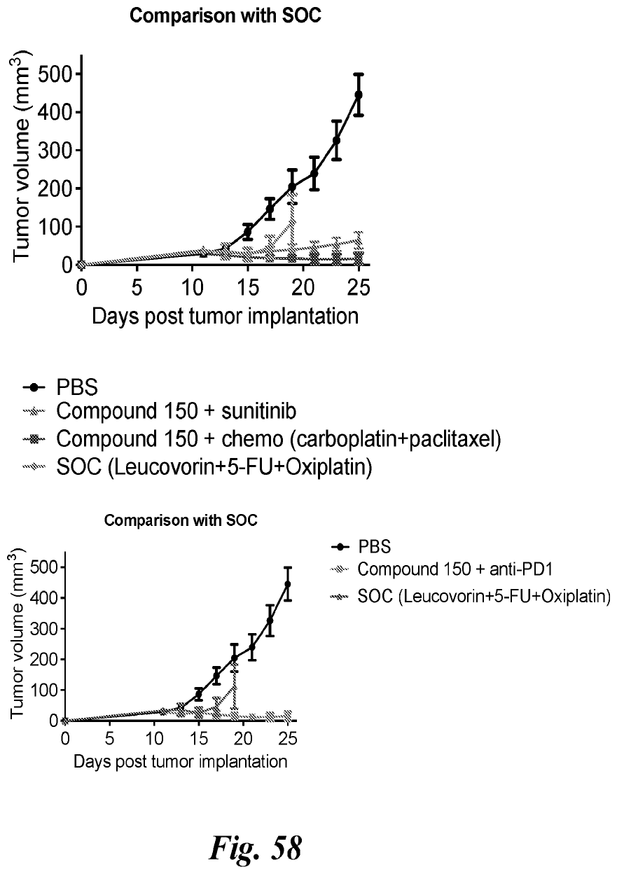

FIG. 58 shows a graph comparing data from FIG. 56 and FIG. 57 to highlight the differences between combination therapies of Compound 150 with anti-PD1/sunitinib/chemotherapy as compared to SOC treatments alone.

### Example 27

### Comparison of Efficacy In Vivo Studies of Compound 150 in Murine Lung Cancer Model Y856 (Cold Tumor Model)

6-8-week-old female C57BL/6 mice (n=5 per cohort) were implanted with 2×10⁶ cells of Y856 murine lung cancer cell line subcutaneously (day 0). At day 11 post implantation, treatment was started with the following test articles: (a) PBS (100 μL/mouse IV)+Anti-DNP/Rham. Ab.; (b) Anti-PD1 (200 μg/mouse IP 3 days/week)+Carboplatin (50 mg/kg IP on day 18 post tumor inoculation)+paclitaxel (36 mg/kg IV once daily from day 18-22 post tumor inoculation); or (c) FDR (Compound 150) (10 nmol/mouse once daily)+Anti-DNP/Rham. Ab.

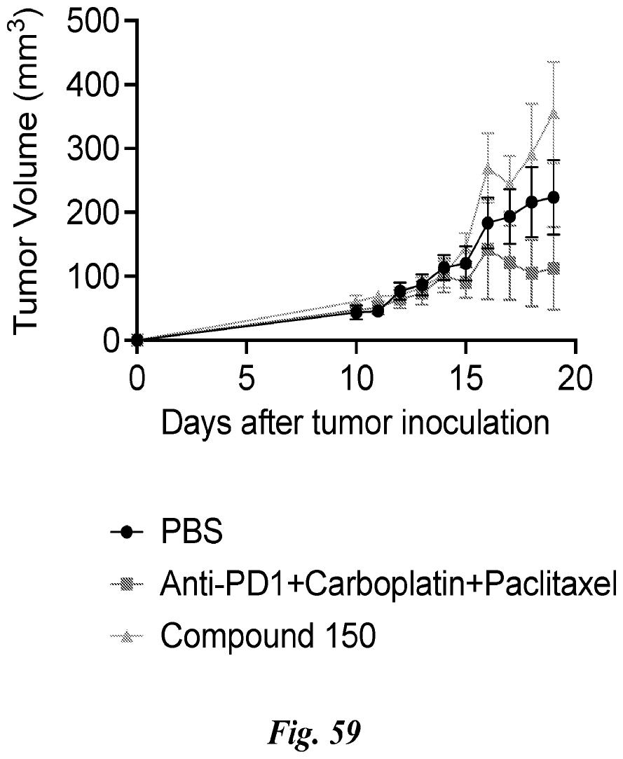

Tumors were measured every other day and mice were sacrificed 25 days post tumor implantation. FIG. 59 shows tumor volume (mm³) versus days post tumor implantation. The PBS and Compound 150 groups also were given an intraperitoneal injection of anti-DNP and anti-Rhamnose antibodies on days 10, 14, 17 and 21 to achieve humanized titer of anti-hapten antibodies throughout the length of the study.

### Example 28

### Efficacy In Vivo Studies of Compound 150 in Murine Renal Cancer Model

6-8-week-old female Balb/c mice were implanted with 2×10⁶ cells of a Renca cancer cell line subcutaneously. Treatment was started 12 days post implantation with the following test articles: (a) PBS (100 μL/mouse IV)+anti-DNP/Rham Ab.; (b) anti-PD1 (200 μg/mouse IP 3 days/week)/PD-L1 (200 μg/mouse IP 3 days/week)+sunitinib (20 mg/kg OG once daily); or (c) FDR (Compound 150) (10 nmol/mouse once daily)+anti-DNP/Rham. Ab., each a daily dose.

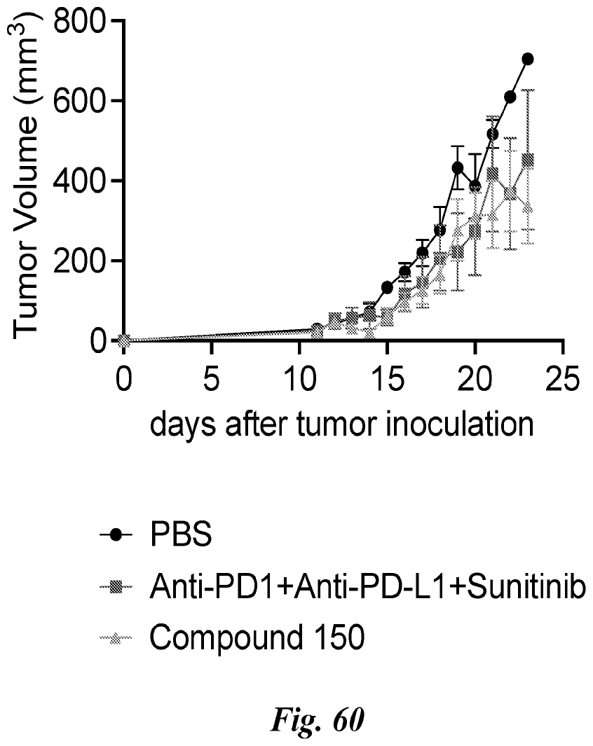

Tumors were measured every other day and mice were sacrificed 25 days post tumor implantation. FIG. 60 shows tumor volume (mm³) versus days post tumor implantation.

### Example 29

### Immune Response Evaluations

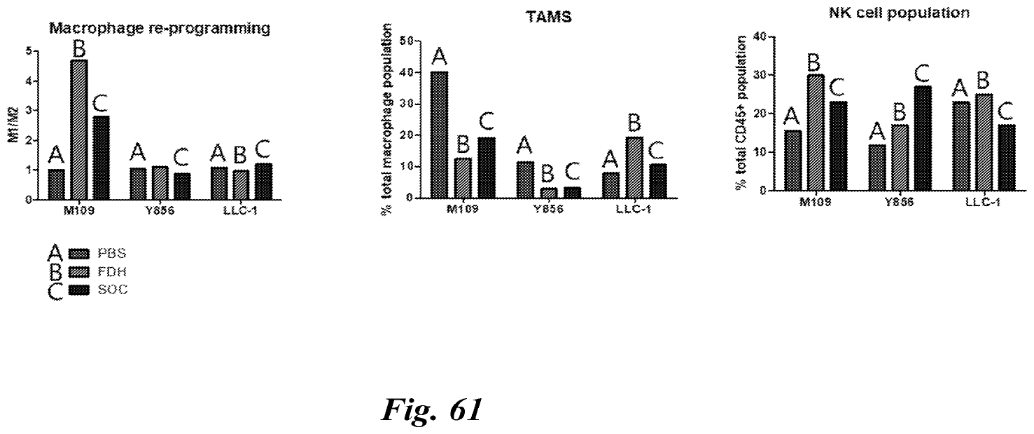

Tumors from the above studies in Balb/c mice using Y856, LLC-1 and M109 cell lines were minced and digested using Tumor Dissociation Kit (Miltenyi Biotec, Bergisch Gladbach, Germany). Single cell suspensions were washed, fixed and stained for the respective immune cell markers before being analyzed by flow cytometry. FIG. 61 shows the comparison of immune cell populations in Y856, LLC-1, and M109 tumors measured following the above-described evaluations. (FDH=Compound 150 and SOC is anti-PD1+chemotheraphy (carboplatin+paclitaxel).

Treatment with Compound 150 caused a significant increase in the population of anti-tumor macrophages and natural killer (NK) cells while reducing the number of tumor associated macrophages (TAMs) in the hot tumor model (M109), but not the cold tumor models (Y8569 and LLC-1). The data supports Compound 150 monotherapies can induce macrophage reprogramming, thus resulting in the reduction in number of TAMs and enhancement of the NK cell population.

1. A conjugate having the formula:
TL-L-Hₙ
or a pharmaceutically acceptable salt thereof, wherein:
TL is a targeting ligand for a target protein on the surface of a virus, a virus-infected cell, a cancer cell, an immune cell, or a fibroblast;
L is a linker;
H is a hapten; and
n is an integer of 2-3; and optionally, wherein at least two of the Hs can each bind a different antibody when brought into contact therewith.

2. The conjugate of claim 1, wherein at least two of the Hs are each bound by an antibody.

3. The conjugate of claim 1 or 2, wherein each H is bound by a different antibody.

4. The conjugate of any one of claims 1-3, wherein each H is independently selected from a rhamnose fragment, an α-galactosyl moiety, a dinitrophenyl fragment, a trinitrophenyl fragment, or a combination thereof.

5. The conjugate of claim 1, wherein n is 2.

6. The conjugate of claim 1, wherein n is 3.

7. The conjugate of any one of claims 1-3, 5, or 6, wherein each H is independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

8. The conjugate of any one of claims 1-3, wherein n is 2, a first H is a DNP fragment, and a second H is a rhamnose fragment.

9. The conjugate of claim 1, wherein at least one H is an influenza virus antigen selected from haemagglutinin and neuraminidase.

10. The conjugate of claim 1, wherein at least one H is a hepatitis antigen selected from L-HBsAg, S-HBsAg, M-HBsAg, and preS.

11. The conjugate of claim 1, wherein at least one H is gp120 or gp160.

12. The conjugate of claim 1, wherein at least one H is a glycoprotein.

13. The conjugate of any one of claims 1-3, 5, 6, or 9-12, wherein the target protein is an envelope protein of a virus or a viral envelope protein on the surface of a virus-infected cell.

14. The conjugate of any one of claims 1-3, 5, 6, or 9-12, wherein the target protein is influenza neuraminidase or influenza hemagglutinin.

15. The conjugate of any one of claims 1-3, 5, 6, or 9-12, wherein the target protein is a respiratory syncytial virus fusion protein F.

16. The conjugate of claims 1-3, 5, 6, or 9-12, wherein the target protein is coronavirus spike protein.

17. The conjugate of claims 1-3, 5, 6, or 9-12, wherein the target protein is hepatitis B virus surface antigen or HBV core antigen.

18. The conjugate of claims 1-3, 5, 6, or 9-12, wherein the target protein is a cell-surface receptor on a cancer cell.

19. The conjugate of claim 1, wherein the target protein is a folate receptor.

20. The conjugate of claim 19, wherein the target protein is folate receptor α or folate receptor β.

21. The conjugate of any one of claims 1-3, 5, 6, or 9-12, wherein the target protein is a prostate-specific membrane antigen.

22. The conjugate of any one of claims 1-3, 5, 6, or 9-12, wherein the target protein is carbonic anhydrase 9.

23. The conjugate of any one of claims 1-3, 5, 6, or 9-12, wherein the target protein is luteinizing hormone releasing hormone receptor.

24. The conjugate of any one of claims 1-3, 5, 6, or 9-12, wherein the target protein is a neurokinin 1 receptor.

25. The conjugate of any one of claims 1-3, 5, 6, or 9-12, wherein the target protein is a cell-surface receptor on a tumor-associated macrophage.

26. The conjugate of any one of claims 1-3, 5, 6, or 9-12, wherein the target protein is a cell-surface receptor on a myeloid-derived suppressor cells.

27. The conjugate of any one of claims 1-3, 5, 6, or 9-12, wherein the target protein is a cell-surface receptor on a cancer-associated fibroblast.

28. The conjugate of any one of claims 1-3, 5, 6, or 9-12, wherein the target protein is a fibroblast activation protein.

29. The conjugate of any one of claims 1-3, 5, 6, or 9-12, wherein the targeting ligand is a neuraminidase inhibitor.

30. The conjugate of any one of claims 1-3, 5, 6, or 9-12, wherein the targeting ligand is an oseltamivir fragment, a zanamivir fragment, a peramivir fragment, or a laninamivir fragment.

31. The conjugate of claim 1, wherein the targeting ligand is a zanamivir fragment.

32. The conjugate of any one of claims 1-3, 5, 6, 9-12, 19, or 20, wherein the targeting ligand is a folic acid fragment or an analog thereof.

33. The conjugate of any one of claims 1-3, 5, 6, 9-12, 12, 19 or 20, wherein the targeting ligand is 5-methyltetrahydrofolate.

34. The conjugate of any one of claims 1-3, 5, 6, 9-12, 19, 20 or 31, wherein L comprises (—CH₂CH₂—O—)ₙ, where n is an integer between and including 1 and 32, a peptide, a peptidoglycan, or a combination of two or more of the foregoing.

35. The conjugate of any one of claims 1-3, 5, 6, 9-12, 19, 20 or 31, wherein L is a branched linker and at least two of the haptens are connected to different branches of the linker, wherein the different branches optionally extend from different atoms of the linker.

36. The conjugate of claim 1, wherein the targeting ligand is a folic acid fragment or a derivative thereof, at least a first H comprises a rhamnose fragment, and at least a second H comprises a dinitrophenyl fragment.

37. The conjugate of any one of claims 1-36 formulated as a prodrug.

38. A conjugate having the formula 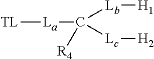
or pharmaceutically acceptable salt thereof, wherein:
TL is a targeting ligand for a target protein on the surface of a virus, a virus-infected cell, a cancer cell, an immune cell, or a fibroblast;
Lₐ, L_{b}, and L_{c} are each a linker, which can be the same or different;
C is a carbon atom;
R₄ is selected from a hydrogen, C₁-C₅ alkyl, C₁-C₅ alkenyl, or C₁-C₅ alkynl group.
H₁ and H₂ are each a hapten; and
optionally, wherein H₁ and H₂ each can bind a different antibody.

39. A conjugate having the formula 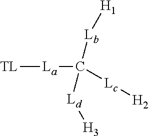
and pharmaceutically acceptable salts thereof, wherein:
TL is a targeting ligand for a target protein on the surface of a virus, a virus-infected cell, a cancer cell, an immune cell, or a fibroblast;
Lₐ, L_{b}, L_{c} and L_{d} are each a linker, which can be the same or different;
C is a carbon atom;
H₁, H₂, and H₃ are each a hapten; and
optionally, wherein each H₁, H₂ and H₃ can each bind a different antibody.

40. The conjugate of claim 38, wherein H₁ and H₂ are each bound by an antibody.

41. The conjugate of claim 39, wherein H₁, H₂, and H₃ are each bound by an antibody.

42. The conjugate of claim 38 or 40, wherein H₁ and H₂ are each independently selected from a rhamnose fragment, an α-galactosyl moiety, a dinitrophenyl fragment, a trinitrophenyl fragment, or a combination thereof.

43. The conjugate of claim 39 or 41, wherein H₁, H₂ and H₃ are each independently selected from a rhamnose fragment, an α-galactosyl moiety, a dinitrophenyl fragment, a trinitrophenyl fragment, or a combination thereof.

44. The conjugate of claim 38 or 40, wherein H₁ or H₂ are each independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

45. The conjugate of claim 38 or 41 wherein H₁, H₂ or H₃ are each independently selected from a rhamnose fragment, an α-galactosyl moiety, a DNP fragment, a TNP fragment, fluorescein, digoxigenin, biotin, or an antigen of a virus selected from diphtheria, zoster virus, human papillomavirus, influenza virus, SARS-COV-2, yellow fever, respiratory syncytial virus, herpes simplex virus, varicella virus, hepatitis A, hepatitis B, hepatitis C, hepatitis D, hepatitis G, rotavirus, mumps virus, tetanus, human immunodeficiency virus, cytomegalovirus, vesicular stomatitis virus, rubella virus, smallpox, monkeypox, poliovirus, dengue virus, and measles virus.

46. The conjugate of claim 38 or 41, wherein H₁ is a DNP fragment, and H₂ is a rhamnose fragment.

47. The conjugate of claim 38 or 39, wherein at least one hapten is an influenza virus antigen selected from haemagglutinin and neuraminidase.

48. The conjugate of claim 38 or 39, wherein at least one hapten is a hepatitis antigen selected from L-HBsAg, S-HBsAg, M-HBsAg, and preS.

49. The conjugate of claim 38 or 39, wherein at least one hapten is gp120 or gp160.

50. The conjugate of claim 38 or 39, wherein at least one hapten is a glycoprotein.

51. The conjugate of any one of claims 38-41, wherein the target protein is an envelope protein of a virus or a viral envelope protein on the surface of a virus-infected cell.

52. The conjugate of any one of claims 38-41, wherein the target protein is influenza neuraminidase or influenza hemagglutinin.

53. The conjugate of any one of claims 38-41, wherein the target protein is a respiratory syncytial virus fusion protein F.

54. The conjugate of any one of claims 38-41, wherein the target protein is coronavirus spike protein.

55. The conjugate of any one of claims 38-41, wherein the target protein is hepatitis B virus surface antigen or HBV core antigen.

56. The conjugate of any one of claims 38-41, wherein the target protein is a cell-surface receptor on a cancer cell.

57. The conjugate of any one of claims 38-41, wherein the target protein is a folate receptor.

58. The conjugate of any one of claims 38-41, wherein the target protein is folate receptor α or folate receptor β.

59. The conjugate of any one of claims 38-41, wherein the target protein is a prostate-specific membrane antigen.

60. The conjugate of any one of claims 38-41, wherein the target protein is carbonic anhydrase 9.

61. The conjugate of any one of claims 38-41, wherein the target protein is a luteinizing hormone releasing hormone receptor.

62. The conjugate of any one of claims 38-41, wherein the target protein is a neurokinin 1 receptor.

63. The conjugate of any one of claims 38-41, wherein the target protein is a cell-surface receptor on a tumor-associated macrophage.

64. The conjugate of any one of claims 38-41, wherein the target protein is a cell-surface receptor on a myeloid-derived suppressor cells.

65. The conjugate of any one of claims 38-41, wherein the target protein is a cell-surface receptor on a cancer-associated fibroblast.

66. The conjugate of any one of claims 38-41, wherein the target protein is a fibroblast activation protein.

67. The conjugate of any one of claims 38-41, wherein the targeting ligand is a neuraminidase inhibitor.

68. The conjugate of any one of claims 38-41, wherein the targeting ligand is an oseltamivir fragment, a zanamivir fragment, a peramivir fragment, or a laninamivir fragment.

69. The conjugate of any one of claims 38-41, wherein the targeting ligand is a zanamivir fragment.

70. The conjugate of any one of claims 38-41, wherein the targeting ligand is a folic acid fragment or an analog thereof.

71. The conjugate of any one of claims 38-41, wherein the targeting ligand is 5-methyltetrahydrofolate.

72. The conjugate of claim 39, wherein at least one of Lₐ, L_{b}, L_{c}, and L_{d} each independently comprise:
(—CH₂CH₂—O—)ₙ, where n is an integer between and including 1 and 32, an alkyl group,
a peptide,
a peptidoglycan, or
a combination of two or more of the foregoing.

73. The conjugate of claim 38, wherein at least one of Lₐ, L_{b}, and L_{c}, each independently comprise:
(—CH₂CH₂—O—)ₙ, where n is an integer between and including 1 and 32, an alkyl group,
a peptide,
a peptidoglycan, or
a combination of two or more of the foregoing.

74. The conjugate of claim 72 or 73, wherein n is an integer between and including 1 and 16.

75. The conjugate of claim 39 or 41, wherein at least one of Lₐ, L_{b}, and L_{c}, and L_{d} comprises a peptide fragment or a peptidoglycan fragment.

76. The conjugate of claim 39 or 40, wherein at least one of Lₐ, L_{b}, and L_{c} comprises a peptide fragment or a peptidoglycan fragment.

77. The conjugate of claim 39 or 41, wherein Lₐ, L_{b} and L_{c} L_{d} each independently comprise a C₂-C₁₈ alkyl group.

78. The conjugate of claim 38 or 40, wherein Lₐ, L_{b} and L_{c} each independently comprise a C₂-C₁₈ alkyl group.

79. A conjugate of the formula: 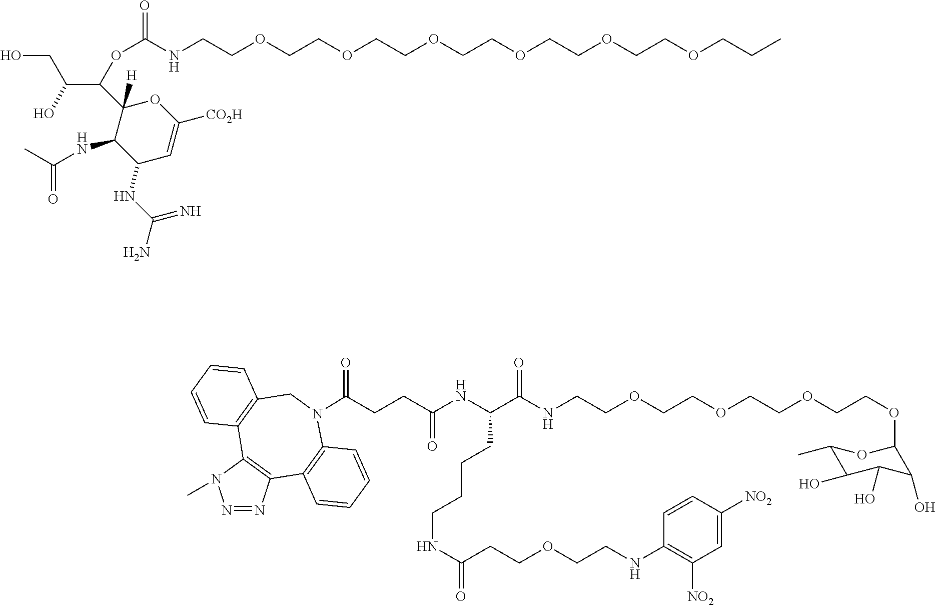
or a pharmaceutically acceptable salt thereof.

80. A conjugate of the formula: 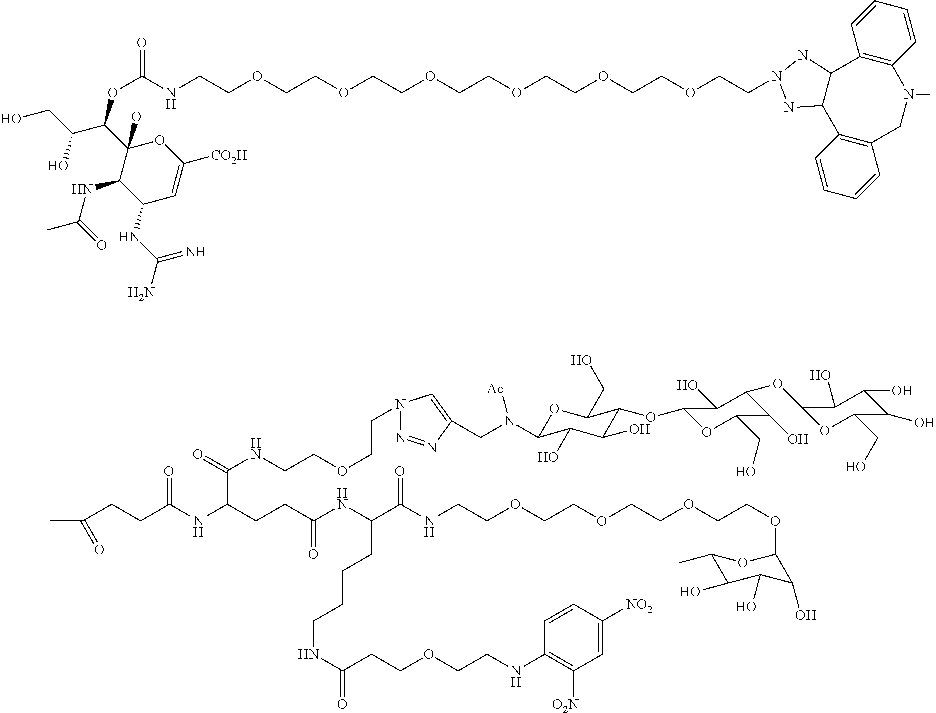
or a pharmaceutically acceptable salt thereof.

81. The conjugate of claim 79 or 80, in which one or more of the —OH groups are independently replaced with a thiol, a phosphate, or a phosphanate ester.

82. The conjugate of claim 79 or 80, in which one or more of the —OH groups are replaced with —OC(═O)R, wherein R is an alkyl group.

83. The conjugate of claim 79 or 80, in which one or more of the —OH groups are replaced with —OC(═O)R, wherein R is a C₁-C₆ alkyl group.

84. The conjugate of claim 79 or 80, in which the amine (—NH₂) group is replaced with —OC(═O)R₂, and R₂ is an alkyl group.

85. The conjugate of claim 79 or 80, in which the amine (—NH₂) group is replaced with —OC(═O)R₂, and R₂ is a C₁-C₆ alkyl group.

86. The conjugate of claim 79 or 80, in which the carboxyl (—COOH) group is replaced with —OC(═O)R₃, wherein R₃ is an alkyl group.

87. The conjugate of claim 79 or 80, in which the carboxyl (—COOH) group is replaced with —OC(═O)R₃, wherein R₃ is a C₁-C₆ alkyl group.

88. A conjugate of the formula: 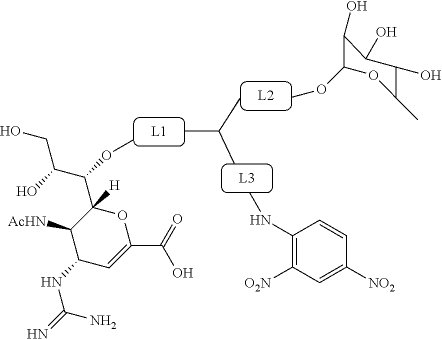 [SMILES: COC1OC(C)C(O)C(O)C1O ; CO[C@H]([C@@H]1OC(C(=O)O)=C[C@H](NC(=N)N)[C@H]1NC(C)=O)[C@H](O)CO ; CNc1ccc([N+](=O)[O-])cc1[N+](=O)[O-]]
or a pharmaceutically acceptable salt thereof, wherein L1, L2 and L3 are linkers.

89. The conjugate of claim 88, wherein one or more of L1, L2 and L3 comprises (—CH₂CH₂—O—)ₙ wherein n is an integer between and including 1 and 16.

90. The conjugate of claim 88 or 89, wherein L1, L2 and L3 each independently comprise a C₂-C₁₈ alkyl group, a peptide fragment, or a peptidoglycan fragment.

91. A conjugate of the formula: 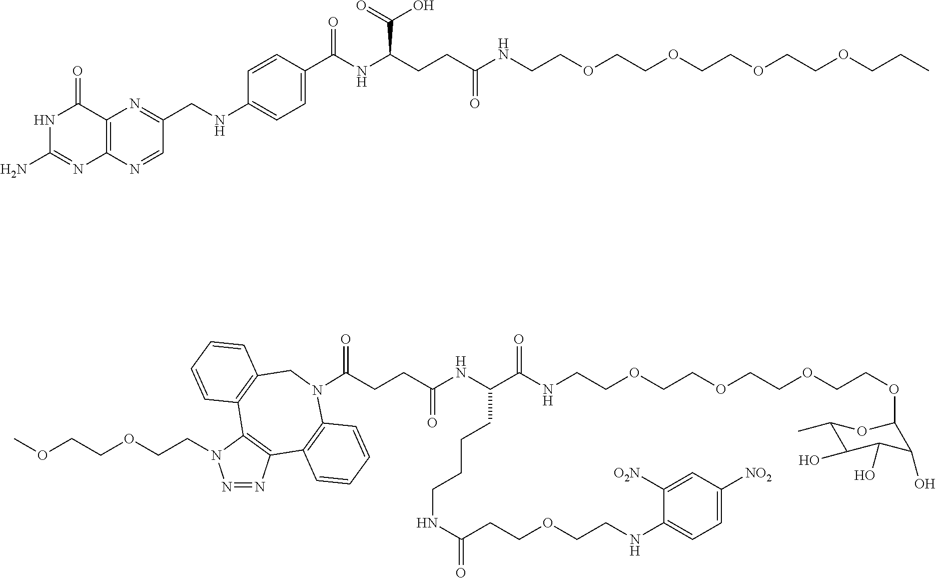
or a pharmaceutically acceptable salt thereof.

92. The conjugate of any one of claims 79 to 91, further complexed to one or more antibodies in vivo.

93. A pharmaceutical composition comprising a conjugate of any one of claims 1-91 and a pharmaceutically acceptable excipient.

94. A method of treating a viral infection in a subject comprising administering to the subject an effective amount of the conjugate of any one of claims 1-91 or a pharmaceutical composition of claim 93.

95. The method of claim 94, further comprising administering to the subject autologous antibodies or allogeneic immunoglobulin G (IgG) antibodies.

96. The method of claim 94, wherein the viral infection is influenza.

97. The method of any one of claims 94-96, wherein the conjugate or the pharmaceutical composition is administered orally.

98. The method of claim 94, wherein the conjugate or the pharmaceutical composition is administered once daily.

99. The method of claim 94, wherein the conjugate or the pharmaceutical composition is administered more than once daily.

100. The method of claim 94, wherein the conjugate or the pharmaceutical composition is administered twice daily.

101. A method of treating cancer in a subject comprising administering to the subject an effective amount of the conjugate of any one of claims 1-91 or a pharmaceutical composition of claim 93.

102. The method of claim 101, further comprising administering to the subject autologous antibodies or allogeneic IgG antibodies.

103. The method of claim 101, wherein the cancer is a hot cancer.

104. The method of claim 101, wherein the cancer is renal cancer, lung cancer, or colorectal cancer.

105. The method of any one of claims 94-104, wherein the conjugate or the pharmaceutical composition is administered orally or intravenously.

106. The method of any one of claims 94-104, wherein the conjugate or the pharmaceutical composition is administered once daily.

107. The method of any one of claims 94-104, further comprising administering a second therapy to the subject, wherein the second therapy comprises chemotherapy, sunitinib, a PD-1 inhibitor, or a PDL-1 inhibitor.

108. A method for activating an immune response in a subject comprising administering to the subject an effective amount of the conjugate of any one of claims 1-91 or a pharmaceutical composition of claim 93.

109. The method of claim 108, wherein the immune response is an innate immune response.

110. The method of claim 108, wherein the immune response is activated in a targeted area of the subject, wherein the targeted area is a tumor microenvironment or a location of a virus replication site.

111. The method of any one of claims 108-110, further comprising administering to the subject autologous antibodies or allogeneic IgG antibodies.

112. The method of claim 110, wherein administration of the effective amount of the conjugate or the pharmaceutical composition induces reprogramming of M2-type macrophages to M1-type macrophages in the targeted area.

113. The conjugate of claim 1, wherein at least two of the Hs can each bind a different antibody when brought into contact with antibodies in vivo.

114. The conjugate of claim 1, wherein two of the Hs can each bind a different antibody when brought into contact with antibodies in vitro.
